(19) United States
(12) Patent Application Publication
(10) Pub. No.: US 2025/0186328 A1
(43) Pub. Date: Jun. 12, 2025

(54) USE OF BOTULINUM TOXINS FOR THE TREATMENT OF PLATYSMA PROMINENCE

(71) Applicant: Allergan, Inc., North Chicago, IL (US)

(72) Inventors: René Marie HOPFINGER, San Diego, CA (US); Elisabeth Ruth LEE, Laguna Beach, CA (US); Warren TONG, Irvine, CA (US)

(73) Assignee: Allergan, Inc., North Chicago, IL (US)

(21) Appl. No.: 18/972,793

(22) Filed: Dec. 6, 2024

Related U.S. Application Data
(60) Provisional application No. 63/607,987, filed on Dec. 8, 2023.

(51) Int. Cl. A61K 8/66; A61Q 19/08

(52) U.S. Cl. CPC A61K 8/66; A61Q 19/08; A61K 2800/91; C12Y 304/24069

(57) ABSTRACT

Provided herein are methods for improving the appearance of platysma prominence associated with platysma muscle activity to a human patient, by administering a composition comprising a botulinum toxin type A to the human patient's platysma muscle. Preferably, the methods involve injection of the composition into the upper segment of the platysma muscle, below the jawline of the human patient and injection of the composition along one or more continuous neck bands on the neck of the human patient.

### CROSS-REFERENCE TO RELATED APPLICATIONS

This application claims the benefit of U.S. Provisional Application No. 63/607,987, filed on Dec. 8, 2023, which is incorporated by reference herein in its entirety.

### 1. FIELD

Provided herein are methods for improving the appearance of platysma prominence associated with platysma muscle activity to a human patient, by administering a composition comprising a botulinum toxin type A to the human patient's platysma muscle.

### 2. BACKGROUND

Botulinum toxins have been widely utilized for therapeutic and aesthetic treatments. Several botulinum toxin type A products have been approved by the United States Food and Drug Administration (FDA) for a variety of therapeutic and cosmetic indications, including onabotulinumtoxinA, approved in 1989 (BOTOX®; Allergan, an AbbVie company, North Chicago, Illinois, USA); abobotulinumtoxinA, approved in 2009 (DYSPORT®; Ipsen Ltd., Slough, UK); incobotulinumtoxinA, approved in 2010 (XEOMIN®; Merz Pharmaceuticals; Frankfurt, Germany); prabotulinumtoxinA, approved in 2019 (JEUVEAU®; Evolus, Inc., Newport Beach, CA, USA); and daxibotulinumtoxinA, approved in 2022 (DAXXIFY®, Revance Therapeutics, Inc., Nashville, TN, USA).

Botulinum toxins have been used in investigational studies to improve the appearance of the lower face and to treat platysmal bands but a number of incidents of adverse events associated with such treatments have been reported, including dysphagia, neck muscle weakness (see, e.g., Obagi and Golubets, 2017, J Drugs Dermatol 16(9):929-930; Dominguez et al., 2016, Emergency Medicine 48(12):551-556; and Witmanowski and Blochowiak, 2020, Postepy Dermatol Alergol 37(6):853-861).

To date, there remains a need for a method that is both effective and safe for treating undesirable aesthetic effects of platysma muscle contraction using botulinum toxins.

Citation of a reference herein shall not be construed as an admission that such is prior art to the present disclosure.

### 3. SUMMARY

The present disclosure provides methods for improving (e.g., temporarily improving) the appearance of platysma prominence associated with platysma muscle activity to a human patient by administering a botulinum toxin type A composition to the human patient's neck. Specifically, the methods administer a botulinum toxin type A composition to a side of a patient's neck if it has only one or only two continuous vertical neck bands. Preferably, the methods involve the injection to the side of the patient's neck of a jawline dose of the botulinum toxin composition below the patient's jawline and neck band dose(s) of the botulinum toxin composition along one or two continuous vertical neck bands. In methods described herein, a continuous vertical neck band is a vertical neck band that extends continuously from the human patient's jawline to a lower neck region.

In one aspect, the present disclosure provides a method for improving (e.g., temporarily improving) the appearance of platysma prominence associated with platysma muscle activity for a specific human patient population, and such a method comprises quantifying the number of continuous vertical neck bands at maximum contraction on the first side of the human patient's neck, wherein a continuous vertical neck band is a vertical neck band that extends continuously from the human patient's jawline to a lower neck region; and then administering a composition comprising a botulinum toxin type A to the first side of the human patient's neck determined as having only one or only two continuous vertical neck bands at maximum contraction.

In a specific embodiment, the present disclosure provides a method for improving (e.g., temporarily improving) the appearance of platysma bands associated with platysma muscle activity to a human patient, comprising quantifying the number of continuous vertical neck bands at maximum contraction on a first side of the human patient's neck, wherein a continuous vertical neck band is a visible vertical neck band that extends continuously from the human patient's jawline to a lower neck region and results in blunting of the jawline (i.e., impacting jawline definition); and then administering a composition comprising a botulinum toxin type A to the first side of the human patient's neck determined as having only one or only two continuous vertical neck bands at maximum contraction.

In a specific embodiment, the present disclosure provides a method for improving (e.g., temporarily improving) the appearance of platysma prominence seen at maximum contraction to a human patient, comprising quantifying the number of continuous vertical neck bands at maximum contraction on a first side of the human patient's neck, wherein a continuous vertical neck band is a visible vertical neck band that extends continuously from the human patient's jawline to a lower neck region and results in blunting of the jawline (i.e., impacting jawline definition); and then administering a composition comprising a botulinum toxin type A to the first side of the human patient's neck determined as having only one or only two continuous vertical neck bands at maximum contraction.

In a specific embodiment, the present disclosure provides a method for improving (e.g., temporarily improving) the appearance of platysma bands prominence seen at maximum contraction to a human patient, comprising quantifying the number of continuous vertical neck bands at maximum contraction on a first side of the human patient's neck, wherein a continuous vertical neck band is a visible vertical neck band that extends continuously from the human patient's jawline to a lower neck region and results in blunting of the jawline (i.e., impacting jawline definition); and then administering a composition comprising a botulinum toxin type A to the first side of the human patient's neck determined as having only one or only two continuous vertical neck bands at maximum contraction.

In a specific embodiment, the present disclosure provides a method for improving (e.g., temporarily improving) the appearance of vertical bands connecting the jaw and neck seen at maximum contraction to a human patient, comprising quantifying the number of continuous vertical neck bands at maximum contraction on a first side of the human patient's neck, wherein a continuous vertical neck band is a visible vertical neck band that extends continuously from the human patient's jawline to a lower neck region and results in blunting of the jawline (i.e., impacting jawline definition); and then administering a composition comprising a botulinum toxin type A to the first side of the human patient's neck determined as having only one or only two continuous vertical neck bands at maximum contraction.

Specific injection paradigms are also provided in the present disclosure. For example, in one aspect, the present disclosure provides a method for improving (e.g., temporarily improving) the appearance of platysma prominence associated with platysma muscle activity to a human patient who has only one continuous vertical neck band at maximum contraction on a first side of the neck, comprising administering a composition comprising a botulinum toxin type A to the first side of the human patient's neck, by injection of: (i) a jawline dose divided among 4 injection sites in the upper segment of platysma muscle, inferior and parallel to the lower mandibular border on the first side, and the 4 injection sites may comprise an anterior injection site in line with the oral commissure and a posterior injection site anterior to the angle of the mandible; and (ii) a neck band dose divided among 5 injection sites along the only continuous vertical neck band.

In a specific embodiment, the present disclosure provides a method for improving (e.g., temporarily improving) the appearance of platysma bands associated with platysma muscle activity to a human patient who has only one continuous vertical neck band at maximum contraction on a first side of the neck, comprising administering a composition comprising a botulinum toxin type A to the first side of the human patient's neck, by injection of: (i) a jawline dose divided among 4 injection sites in the upper segment of platysma muscle, inferior and parallel to the lower mandibular border on the first side, and the 4 injection sites may comprise an anterior injection site in line with the oral commissure and a posterior injection site anterior to the angle of the mandible; and (ii) a neck band dose divided among 5 injection sites along the only continuous vertical neck band; wherein a continuous vertical neck band is a visible vertical neck band that extends continuously from the human patient's jawline to a lower neck region and results in blunting of the jawline (i.e., impacting jawline definition).

In a specific embodiment, the present disclosure provides a method for improving (e.g., temporarily improving) the appearance of platysma prominence seen at maximum contraction to a human patient who has only one continuous vertical neck band at maximum contraction on a first side of the neck, comprising administering a composition comprising a botulinum toxin type A to the first side of the human patient's neck, by injection of: (i) a jawline dose divided among 4 injection sites in the upper segment of platysma muscle, inferior and parallel to the lower mandibular border on the first side, and the 4 injection sites may comprise an anterior injection site in line with the oral commissure and a posterior injection site anterior to the angle of the mandible; and (ii) a neck band dose divided among 5 injection sites along the only continuous vertical neck band; wherein a continuous vertical neck band is a visible vertical neck band that extends continuously from the human patient's jawline to a lower neck region and results in blunting of the jawline (i.e., impacting jawline definition).

In a specific embodiment, the present disclosure provides a method for improving (e.g., temporarily improving) the appearance of platysma bands prominence seen at maximum contraction to a human patient who has only one continuous vertical neck band at maximum contraction on a first side of the neck, comprising administering a composition comprising a botulinum toxin type A to the first side of the human patient's neck, by injection of: (i) a jawline dose divided among 4 injection sites in the upper segment of platysma muscle, inferior and parallel to the lower mandibular border on the first side, and the 4 injection sites may comprise an anterior injection site in line with the oral commissure and a posterior injection site anterior to the angle of the mandible; and (ii) a neck band dose divided among 5 injection sites along the only continuous vertical neck band; wherein a continuous vertical neck band is a visible vertical neck band that extends continuously from the human patient's jawline to a lower neck region and results in blunting of the jawline (i.e., impacting jawline definition).

In a specific embodiment, the present disclosure provides a method for improving (e.g., temporarily improving) the appearance of vertical bands connecting the jaw and neck seen at maximum contraction to a human patient who has only one continuous vertical neck band at maximum contraction on a first side of the neck, comprising administering a composition comprising a botulinum toxin type A to the first side of the human patient's neck, by injection of: (i) a jawline dose divided among 4 injection sites in the upper segment of platysma muscle, inferior and parallel to the lower mandibular border on the first side, and the 4 injection sites may comprise an anterior injection site in line with the oral commissure and a posterior injection site anterior to the angle of the mandible; and (ii) a neck band dose divided among 5 injection sites along the only continuous vertical neck band; wherein a continuous vertical neck band is a visible vertical neck band that extends continuously from the human patient's jawline to a lower neck region and results in blunting of the jawline (i.e., impacting jawline definition).

In another aspect, the present disclosure provides a method for improving (e.g., temporarily improving) the appearance of platysma prominence associated with platysma muscle activity to a human patient who has only two continuous vertical neck bands at maximum contraction on a first side of the neck, comprising administering a composition comprising a botulinum toxin type A to the first side of the human patient's neck, by injection of: (i) a jawline dose divided among 4 injection sites in the upper segment of platysma muscle, inferior and parallel to the lower mandibular border on the first side, and the 4 injection sites may optionally comprise an anterior injection site in line with the oral commissure and a posterior injection site anterior to the angle of the mandible; (ii) a first neck band dose divided among 5 injection sites along a first continuous vertical neck band; and (iii) a second neck band dose divided among 5 injection sites along a second continuous vertical neck band; and wherein the administering step delivers a total unilateral dose range of about 15 units to about 20 units of botulinum toxin type A to the first side of the human patient's neck.

In a specific embodiment, the present disclosure provides a method for improving (e.g., temporarily improving) the appearance of platysma bands associated with platysma muscle activity to a human patient who has only two continuous vertical neck bands at maximum contraction on a first side of the neck, comprising administering a composition comprising a botulinum toxin type A to the first side of the human patient's neck, by injection of: (i) a jawline dose divided among 4 injection sites in the upper segment of platysma muscle, inferior and parallel to the lower mandibular border on the first side, and the 4 injection sites may optionally comprise an anterior injection site in line with the oral commissure and a posterior injection site anterior to the angle of the mandible; (ii) a first neck band dose divided among 5 injection sites along a first continuous vertical neck band; and (iii) a second neck band dose divided among 5 injection sites along a second continuous vertical neck band; wherein a continuous vertical neck band is a visible vertical neck band that extends continuously from the human patient's jawline to a lower neck region and results in blunting of the jawline, and wherein the administering step delivers a total unilateral dose range of about 15 units to about 20 units of botulinum toxin type A to the first side of the human patient's neck.

In a specific embodiment, the present disclosure provides a method for improving (e.g., temporarily improving) the appearance of platysma bands associated with platysma muscle activity to a human patient who has only two continuous vertical neck bands at maximum contraction on a first side of the neck, comprising administering a composition comprising a botulinum toxin type A to the first side of the human patient's neck, by injection of: (i) a jawline dose divided among 4 injection sites in the upper segment of platysma muscle, inferior and parallel to the lower mandibular border on the first side, and the 4 injection sites may optionally comprise an anterior injection site in line with the oral commissure and a posterior injection site anterior to the angle of the mandible; (ii) a first neck band dose divided among 5 injection sites along a first continuous vertical neck band; and (iii) a second neck band dose divided among 5 injection sites along a second continuous vertical neck band; wherein the administering step delivers a total unilateral dose range of about 15 units to about 20 units of botulinum toxin type A to the first side of the human patient's neck.

In a specific embodiment, the present disclosure provides a method for improving (e.g., temporarily improving) the appearance of platysma prominence seen at maximum contraction to a human patient who has only two continuous vertical neck bands at maximum contraction on a first side of the neck, comprising administering a composition comprising a botulinum toxin type A to the first side of the human patient's neck, by injection of: (i) a jawline dose divided among 4 injection sites in the upper segment of platysma muscle, inferior and parallel to the lower mandibular border on the first side, and the 4 injection sites may optionally comprise an anterior injection site in line with the oral commissure and a posterior injection site anterior to the angle of the mandible; (ii) a first neck band dose divided among 5 injection sites along a first continuous vertical neck band; and (iii) a second neck band dose divided among 5 injection sites along a second continuous vertical neck band; wherein a continuous vertical neck band is a visible vertical neck band that extends continuously from the human patient's jawline to a lower neck region and results in blunting of the jawline, and wherein the administering step delivers a total unilateral dose range of about 15 units to about 20 units of botulinum toxin type A to the first side of the human patient's neck.

In a specific embodiment, the present disclosure provides a method for improving (e.g., temporarily improving) the appearance of platysma bands prominence seen at maximum contraction to a human patient who has only two continuous vertical neck bands at maximum contraction on a first side of the neck, comprising administering a composition comprising a botulinum toxin type A to the first side of the human patient's neck, by injection of: (i) a jawline dose divided among 4 injection sites in the upper segment of platysma muscle, inferior and parallel to the lower mandibular border on the first side, and the 4 injection sites may optionally comprise an anterior injection site in line with the oral commissure and a posterior injection site anterior to the angle of the mandible; (ii) a first neck band dose divided among 5 injection sites along a first continuous vertical neck band; and (iii) a second neck band dose divided among 5 injection sites along a second continuous vertical neck band; wherein a continuous vertical neck band is a visible vertical neck band that extends continuously from the human patient's jawline to a lower neck region and results in blunting of the jawline, and wherein the administering step delivers a total unilateral dose range of about 15 units to about 20 units of botulinum toxin type A to the first side of the human patient's neck.

In a specific embodiment, the present disclosure provides a method for improving (e.g., temporarily improving) the appearance of vertical bands connecting the jaw and neck seen at maximum contraction to a human patient who has only two continuous vertical neck bands at maximum contraction on a first side of the neck, comprising administering a composition comprising a botulinum toxin type A to the first side of the human patient's neck, by injection of: (i) a jawline dose divided among 4 injection sites in the upper segment of platysma muscle, inferior and parallel to the lower mandibular border on the first side, and the 4 injection sites may optionally comprise an anterior injection site in line with the oral commissure and a posterior injection site anterior to the angle of the mandible; (ii) a first neck band dose divided among 5 injection sites along a first continuous vertical neck band; and (iii) a second neck band dose divided among 5 injection sites along a second continuous vertical neck band; wherein a continuous vertical neck band is a visible vertical neck band that extends continuously from the human patient's jawline to a lower neck region and results in blunting of the jawline, and wherein the administering step delivers a total unilateral dose range of about 15 units to about 20 units of botulinum toxin type A to the first side of the human patient's neck.

In another aspect, the present disclosure provides a method for improving (e.g., temporarily improving) the appearance of platysma prominence associated with platysma muscle activity to a human patient who has only one continuous vertical neck band at maximum contraction on a first side of the neck and only two continuous vertical neck bands at maximum contraction on a second side of the neck, the method comprising administering a composition comprising a botulinum toxin type A to the human patient's neck, by injection of: (i) a first jawline dose divided among 4 injection sites in the upper segment of platysma muscle, inferior and parallel to the lower mandibular border, and the 4 injection sites may optionally comprise an anterior injection site in line with the oral commissure and a posterior injection site anterior to the angle of the mandible; (ii) a first neck band dose divided among 5 injection sites along the only continuous vertical neck band on the first side; (iii) a second jawline dose divided among 4 injection sites in the upper segment of platysma muscle, inferior and parallel to the lower mandibular border, and the 4 injection sites may optionally comprise an anterior injection site in line with the oral commissure and a posterior injection site anterior to the angle of the mandible; (iv) a first neck band dose divided among 5 injection sites along a first continuous vertical neck band on the second side; and (v) a second neck band dose divided among 5 injection sites along a second continuous vertical neck band on the second side.

In a specific embodiment, the present disclosure provides a method for improving (e.g., temporarily improving) the appearance of platysma bands associated with platysma muscle activity to a human patient who has only one continuous vertical neck band at maximum contraction on a first side of the neck and only two continuous vertical neck bands at maximum contraction on a second side of the neck, the method comprising administering a composition comprising a botulinum toxin type A to the human patient's neck, by injection of: (i) a first jawline dose divided among 4 injection sites in the upper segment of platysma muscle, inferior and parallel to the lower mandibular border on the first side, and the 4 injection sites may optionally comprise an anterior injection site in line with the oral commissure and a posterior injection site anterior to the angle of the mandible; (ii) a first neck band dose divided among 5 injection sites along the only continuous vertical neck band on the first side; (iii) a second jawline dose divided among 4 injection sites in the upper segment of platysma muscle, inferior and parallel to the lower mandibular border on the second side, and the 4 injection sites may optionally comprise an anterior injection site in line with the oral commissure and a posterior injection site anterior to the angle of the mandible; (iv) a first neck band dose divided among 5 injection sites along a first continuous vertical neck band on the second side; and (v) a second neck band dose divided among 5 injection sites along a second continuous vertical neck band on the second side; wherein a continuous vertical neck band is a visible vertical neck band that extends continuously from the human patient's jawline to a lower neck region and results in blunting of the jawline.

In a specific embodiment, the present disclosure provides a method for improving (e.g., temporarily improving) the appearance of platysma prominence seen at maximum contraction to a human patient who has only one continuous vertical neck band at maximum contraction on a first side of the neck and only two continuous vertical neck bands at maximum contraction on a second side of the neck, the method comprising administering a composition comprising a botulinum toxin type A to the human patient's neck, by injection of: (i) a first jawline dose divided among 4 injection sites in the upper segment of platysma muscle, inferior and parallel to the lower mandibular border on the first side, and the 4 injection sites may optionally comprise an anterior injection site in line with the oral commissure and a posterior injection site anterior to the angle of the mandible; (ii) a first neck band dose divided among 5 injection sites along the only continuous vertical neck band on the first side; (iii) a second jawline dose divided among 4 injection sites in the upper segment of platysma muscle, inferior and parallel to the lower mandibular border on the second side, and the 4 injection sites may optionally comprise an anterior injection site in line with the oral commissure and a posterior injection site anterior to the angle of the mandible; (iv) a first neck band dose divided among 5 injection sites along a first continuous vertical neck band on the second side; and (v) a second neck band dose divided among 5 injection sites along a second continuous vertical neck band on the second side; wherein a continuous vertical neck band is a visible vertical neck band that extends continuously from the human patient's jawline to a lower neck region and results in blunting of the jawline.

In a specific embodiment, the present disclosure provides a method for improving (e.g., temporarily improving) the appearance of platysma bands prominence seen at maximum contraction to a human patient who has only one continuous vertical neck band at maximum contraction on a first side of the neck and only two continuous vertical neck bands at maximum contraction on a second side of the neck, the method comprising administering a composition comprising a botulinum toxin type A to the human patient's neck, by injection of: (i) a first jawline dose divided among 4 injection sites in the upper segment of platysma muscle, inferior and parallel to the lower mandibular border on the first side, and the 4 injection sites may optionally comprise an anterior injection site in line with the oral commissure and a posterior injection site anterior to the angle of the mandible; (ii) a first neck band dose divided among 5 injection sites along the only continuous vertical neck band on the first side; (iii) a second jawline dose divided among 4 injection sites in the upper segment of platysma muscle, inferior and parallel to the lower mandibular border on the second side, and the 4 injection sites may optionally comprise an anterior injection site in line with the oral commissure and a posterior injection site anterior to the angle of the mandible; (iv) a first neck band dose divided among 5 injection sites along a first continuous vertical neck band on the second side; and (v) a second neck band dose divided among 5 injection sites along a second continuous vertical neck band on the second side; wherein a continuous vertical neck band is a visible vertical neck band that extends continuously from the human patient's jawline to a lower neck region and results in blunting of the jawline.

In a specific embodiment, the present disclosure provides a method for improving (e.g., temporarily improving) the appearance of vertical bands connecting the jaw and neck seen at maximum contraction to a human patient who has only one continuous vertical neck band at maximum contraction on a first side of the neck and only two continuous vertical neck bands at maximum contraction on a second side of the neck, the method comprising administering a composition comprising a botulinum toxin type A to the human patient's neck, by injection of: (i) a first jawline dose divided among 4 injection sites in the upper segment of platysma muscle, inferior and parallel to the lower mandibular border on the first side, and the 4 injection sites may optionally comprise an anterior injection site in line with the oral commissure and a posterior injection site anterior to the angle of the mandible; (ii) a first neck band dose divided among 5 injection sites along the only continuous vertical neck band on the first side; (iii) a second jawline dose divided among 4 injection sites in the upper segment of platysma muscle, inferior and parallel to the lower mandibular border on the second side, and the 4 injection sites may optionally comprise an anterior injection site in line with the oral commissure and a posterior injection site anterior to the angle of the mandible; (iv) a first neck band dose divided among 5 injection sites along a first continuous vertical neck band on the second side; and (v) a second neck band dose divided among 5 injection sites along a second continuous vertical neck band on the second side; wherein a continuous vertical neck band is a visible vertical neck band that extends continuously from the human patient's jawline to a lower neck region and results in blunting of the jawline.

In another aspect, the present disclosure provides a method for the treatment of the appearance of platysma bands associated with platysma muscle activity in a human patient using a composition comprising a botulinum toxin type A, wherein the treatment comprises intramuscularly administering to a first side of the human patient's neck: a) a first jawline dose divided among 4 injection sites in the upper segment of platysma muscle on the first side of the human patient's neck, wherein the first jawline dose is about 8 units of botulinum toxin type A; and b) a first neck band dose divided among 5 injection sites along a first platysma band on the first side of the human patient's neck, wherein the first neck band dose is about 5 units of botulinum toxin type A; and wherein the appearance of the platysma bands in the human patient is safely and effectively treated.

In specific embodiments, the method further comprises intramuscularly administering to a second side of the patient's neck: a) a second jawline dose divided among 4 injection sites in the upper segment of platysma muscle on the second side of the human patient's neck, wherein the second jawline dose is about 8 units of botulinum toxin type A; and b) a second neck band dose divided among 5 injection sites along a first platysma band on the second side of the human patient's neck, wherein the second neck band dose is about 5 units of botulinum toxin type A.

In specific embodiments, the method further comprises intramuscularly administering to at least one of the first or second side of the human patient's neck a third neck band dose divided among 5 injection sites along a third platysma band, wherein the third neck band dose is about 5 units of botulinum toxin type A.

In specific embodiments, the method further comprises administering a fourth neck band dose divided among 5 injection sites along a fourth platysma band, wherein the fourth neck band dose is about 5 units of botulinum toxin type A, and wherein the human patient has two platysma bands located on each of the first and second sides of the human patient's neck.

In specific embodiments, the administering step delivers a total bilateral dose of 26 units of botulinum toxin type A. In specific embodiments, the administering step delivers a total bilateral dose of 31 units of botulinum toxin type A. In specific embodiments, the administering step delivers a total bilateral dose of 36 units of botulinum toxin type A.

In specific embodiments, the first platysma band on the first side, the first platysma band on the second side, the third platysma band on the at least one of the first or second side of the human patient's neck, and/or the fourth platysma band are visible continuous vertical neck bands at maximum contraction that extend continuously from the human patient's jawline to a lower neck region and result in blunting of the jawline.

In specific embodiments, the 4 jawline injection sites are administered inferior and parallel to the patient's lower mandibular border.

In specific embodiments, the 4 jawline injection sites are administered approximately 1-2 cm inferior and parallel to the patient's lower mandibular border.

In specific embodiments, the first jawline injection site is an anterior injection site in line with the patient's oral commissure, the fourth jawline injection site is a posterior injection site anterior to the angle of the mandible, and the second and third jawline injection sites are equidistant between the first and fourth jawline injection sites.

In specific embodiments, the first injection site of the first, second, third, or fourth neck band dose is administered approximately 1-2 cm inferior to the jawline injection sites.

In specific embodiments, the neck band injection sites are spaced approximately 1-2 cm apart.

In specific embodiments, the treatment provides temporary improvement in the appearance of the platysma bands.

### 4. BRIEF DESCRIPTION OF THE FIGURES

FIG. 1. Illustration of Platysma Prominence at maximum contraction at different levels of severity that accompanies the Allergan Platysma Prominence Scale (APPS) used to assess platysma prominence severity of a human patient (i.e. subject or participant). The APPS is independently used by clinicians or investigators (referred to as C-APPS) and subjects (referred to as P-APPS) to assess platysma prominence severity.

FIGS. 2A-2B. Schematic of injection sites for treatment of moderate platysma prominence (FIG. 2B) and for treatment of severe platysma prominence (FIG. 2A).

FIGS. 3A-3B. Responder rate (%) with 95% Confidence Intervals (CIs) of participants who achieved at least a 1-grade improvement from baseline based on investigator's assessment using the C-APPS (FIG. 3A) or subject's self-assessment using the P-APPS (FIG. 3B) at maximum contraction over a 120-day period after administration of a high dose (HD) or low dose (LD) of a botulinum toxin type A (BOTOX). HD: high dose. LD: low dose. CI: confidence interval.

FIG. 4. Study schematic of Phase 3 clinical studies described in Example 2, wherein the two phase 3 studies (study 1, also referred to as the M21-309 study or the first phase 3 clinical study, and study 2, also referred to as the M21-310 study or the second phase 3 clinical study) occurred in the first 4 months, and for eligible subjects, the 8-month Open Label extended treatment study started on the exit day (Day 120) of the first phase 3 clinical study.

FIG. 5. Response rate (%) of participants in the ITT population of the first phase 3 clinical study (i.e., study 1, or the M21-309 study) who achieved Grade 1 (Minimal) or Grade 2 (Mild) and at least a 2-grade improvement from baseline based on both investigator's assessment using the C-APPS and subject's self-assessment using the P-APPS at maximum contraction at primary timepoint Day 14. *p<0.0001, or the mITT population of the first phase 3 clinical study who achieved at least a 2-grade improvement from baseline based on investigator's assessment using the C-APPS or subject's self-assessment using the P-APPS at maximum contraction at primary timepoint Day 14. Error bars are 95% confidence interval. ITT refers to the Intent To Treat population, which comprised all randomized subjects, and mITT refers to the Modified Intent To Treat population, which comprised all randomized subjects having a baseline summary score of ≥19 for the Appearance of Neck and Lower Face Questionnaire (ANLFQ): Impacts questionnaire. A comparable Response Rate (%) was obtained for the ITT and mITT populations in the second phase 3 clinical study (i.e., study 2, or the M21-310 study) (see Table 8, FIGS. 6A-6C).

FIGS. 6A-6C. Response rate (%) of participants in the ITT populations of the M21-309 and M21-310 studies, respectively, who achieved Grade 1 (Minimal) or Grade 2 (Mild) and at least a 2-grade improvement from baseline based on both investigator's assessment using the C-APPS and subject's self-assessment using the P-APPS at maximum contraction from day 14 to day 120 (FIG. 6A); and in the mITT populations of the M21-309 and M21-310 studies, respectively, who achieved at least a 2-grade improvement from baseline based on either investigator's assessment using the C-APPS (FIG. 6B) or subject's self-assessment using the P-APPS (FIG. 6C) at maximum contraction from day 14 to day 120. Error bars are 95% confidence interval. Boxed: Day 14 primary timepoint. *: Graph includes data for responder with the following definition: achievement of Grade 1 or 2 (Minimal or Mild) and at least a 2-grade improvement (ITT).

FIGS. 7A-7B. Response rate (%) of participants in the ITT populations of the M21-309 and M21-310 studies, respectively, who achieved minimal or mild platysma prominence based on investigator's assessment using the C-APPS (FIG. 7A) or subject's self-assessment using the P-APPS (FIG. 7B) at maximum contraction from day 14 to day 120. Error bars are 95% confidence interval. Boxed: Day 14 primary timepoint.

FIGS. 8A-8B. Responder rate (%) of participants in the ITT population who achieved minimal or mild platysma prominence based on investigator's assessment using the C-APPS ( . . . dotted line) or subject's self-assessment using the P-APPS (- - - dashed line), or minimal or mild platysma prominence and at least a 2-grade improvement based on both investigator's assessment using the C-APPS and subject's self-assessment using the P-APPS (composite, solid line), at maximum contraction from day 14 to day 120 in the M21-309 study (FIG. 8A) and in the M21-310 study (FIG. 8B).

FIGS. 9A-9C. Percentage (%) of Participants in the ITT populations of the M21-309 and M21-310 studies, respectively, with Responses of: (1) “Satisfied” or “Very Satisfied” over time in terms of their satisfaction with the effect of the treatment on item 5 of the Appearance of Neck and Lower Face Questionnaire (ANLFQ): Satisfaction questionnaire (FIG. 9A); (2) “Not at all Bothered” or “A Little Bothered” by their jawline in the Bother Assessment Scale-Platysma Prominence-(BAS-PP) questionnaire (Item 2) over time (FIG. 9B); or (3) “Not at all Bothered” or “A Little Bothered” by their vertical neck bands over time in the BAS-PP questionnaire (Item 1) (FIG. 9C). Error bars are 95% confidence interval. Boxed: Day 14 primary timepoint.

### 5. DETAILED DESCRIPTION

Although botulinum toxins have been used in investigational studies to improve the appearance of the lower face and to treat platysmal bands, there remains a need for improved methods for using botulinum toxin administration that are both effective and safe for treating undesirable aesthetic effects of platysma muscle contraction.

The development of a new scale for assessing platysma prominence or patient selection criteria is essential for improving the safety and effectiveness of botulinum toxin treatments. It allows for individualized treatment plans, enhances safety by reducing the risk of complications, and improves treatment outcomes, all while providing a standardized framework for practice in this area. The platysma muscle's anatomy and activity levels can vary significantly among individuals. Creating a new effective scale or patient selection criteria that accommodates this diversity while remaining simple and practical is a complex task. The platysma muscle is dynamic, and its activity changes with facial expressions and movements. The platysma muscle is interconnected with other facial muscles, and its function is interrelated with them. Developing a scale that accounts for this dynamic and interactive nature, as well as static muscle prominence, is a challenge. In addition, assessing platysma prominence often involves a degree of subjectivity, as different practitioners may have varying interpretations of muscle prominence based on visual or tactile examination. A scale for platysma prominence that helps with achieving objectivity and consistency in the assessment and is practical for clinical use, ensuring that it can be easily integrated into the workflow of practitioners, as well as accompanying dosing methods, are much needed in the art. The present disclosure addresses such a need and provides related treatment methods.

Specifically, the methods provided herein administer a botulinum toxin type A composition to a side of a patient's neck if it has only one or only two continuous vertical neck bands. Such patients may further have one or more non-continuous vertical neck bands. Preferably, the methods involve the injection to the side of the patient's neck of a jawline dose of the botulinum toxin composition below the patient's jawline and neck band dose(s) of the botulinum toxin composition along one or two continuous bands. More preferably, the jawline dose and the neck band dose(s) are each divided among several injection sites and the neck band dose(s) are lower than the jawline dose. For example, the jawline dose can be in a total of 8 units of botulinum toxin type A divided among 4 injection sites, preferably with each injection being about 2 units, while the neck band dose can be in a total of 5 units of botulinum toxin type A divided among 5 injection sites, preferably with each injection being about 1 unit. Further detailed descriptions of the methods and specific injection paradigms can be found in Section 5.3 below. The various methods described in this disclosure provide a holistic approach that has a favorable benefit/risk profile for treating undesirable aesthetic effects of platysma muscle contraction (see more details below).

The platysma muscle complex is composed of 2 separate, superficial muscle sheets that originate from the fascia of the upper thoracic region and pass upward over both sides of the neck, crossing over the mandibular border and inserting into the overlying skin of the lower face. When relaxed, the platysma muscle smoothly drapes the jawline, neck, and clavicle to create a firm, fitted, and uniform appearance.

As the largest mimetic muscle of the face and neck, platysma contraction serves a role in communicating an individual's emotional state in social interactions. Specifically, platysma contraction may be associated with a variety of facial expressions conveying negative emotions such as fear, disgust, and aggression. Functionally, the platysma works in concert with the risorius muscle to retract the corners of the mouth and assists in depression of the labial commissures, possibly contributing to resting skin tension lines at the labial commissures. Below the mandibular border, platysma contraction tenses the skin along the inferior margin of the mandible causing a blunting effect as the concavity between the jaw and the side of the neck is reduced. Along the length of the neck, platysma contraction may produce distinct vertical banding, which typically becomes more apparent with age.

Over time, the repetitive contractions of platysma, along with degenerative changes due to aging (e.g., cervical skin thinning, cervical adipose tissue loss, and platysma muscle diastasis), may lead to an unsightly appearance of the platysma muscle that demonstrates primarily as vertical bands on the neck, and may include blunting of the jawline (Le Louarn et al., 2007, Aesthetic Plast Surg 31(3):213-218; Brandt and Bellman, 1998, Dermatol Surg 24(11):1232-1234; Hwang et al., 2017, J Craniofac Surg 28(2):539-542; and Le Louarn, 2016, Ann Chir Plast Esthet 61(2):101-109).

“Platysma prominence” is used herein to represent the undesirable aesthetic effects of platysma muscle contraction, including, for example, vertical bands on the neck, also referred to as platysma bands or platysmal bands, and blunting of the jawline. Platysma prominence arises largely from activity of the platysma muscles. Platysma prominence manifests when the platysma muscle is contracted through normal movement, such as talking or smiling, imparting an aged appearance. Forceful contraction of the platysma tenses the skin on the neck, which may result in blunting of the jawline and vertical neck bands. Platysma prominence may create a false perception of an individual's emotional state in social interactions in concert with other degenerative changes. It is perceived as a sign of aging in an individual whose facial appearance may otherwise appear attractively trim, taut, and smooth. The location, size, and presentation of the platysma prominence varies among individuals.

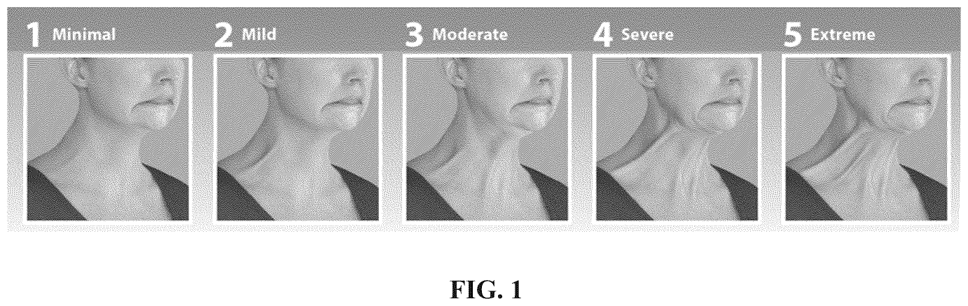

Platysma prominence described in this disclosure can be classified into multiple grades according to a platysma prominence scale, assessed at maximum contraction. In various embodiments, the multiple grades of the platysma prominence scale comprise at least a grade corresponding to moderate platysma prominence and a grade corresponding to severe platysma prominence. In certain embodiments, the multiple grades of the platysma prominence scale comprise at least a grade corresponding to minimal platysma prominence, a grade corresponding to mild platysma prominence, a grade corresponding to moderate platysma prominence, and a grade corresponding to severe platysma prominence. In certain embodiments, the multiple grades of the platysma prominence scale comprise a grade corresponding to minimal platysma prominence, a grade corresponding to mild platysma prominence, a grade corresponding to moderate platysma prominence, a grade corresponding to severe platysma prominence, and a grade corresponding to extreme platysma prominence. In a specific embodiment, the multiple grades of the platysma prominence scale are 5 grades corresponding to minimal platysma prominence, mild platysma prominence, moderate platysma prominence, severe platysma prominence, and extreme platysma prominence, respectively. In a specific embodiment, platysma prominence described in this disclosure can be classified into 5 grades according to the Allergan Platysma Prominence Scale (APPS) illustrated by Table 1 below, assessed at maximum contraction and illustrated in FIG. 1. The 5 grades listed in Table 1 increase by the severity of platysma prominence from Grade 1 to Grade 5. In specific embodiments, the platysma prominence scale is a platysma band severity grading scale. In a preferred embodiment, the platysma prominence scale is a platysma band severity grading scale according to which platysma band severity assessed at maximum contraction is classified into 5 grades, wherein grade 1 corresponds to minimal platysma band severity, which is associated with no visible neck bands and no blunting of the jawline (i.e., no impact to jawline definition), grade 2 corresponds to mild platysma band severity, which is associated with visible neck bands and no blunting of the jawline (i.e., no impact to jawline definition), grade 3 corresponds to moderate platysma band severity, which is associated with 1 visible continuous neck band that results in blunting of the jawline (i.e., impacting jawline definition), grade 4 corresponds to severe platysma band severity, which is associated with 2 visible continuous neck bands that result in blunting of the jawline (i.e., impacting jawline definition), and grade 5 corresponds to extreme platysma band severity, which is associated with 3 or more visible neck bands that result in blunting of the jawline (i.e., impacting jawline definition).

TABLE 1
The Allergan Platysma Prominence Scale
(APPS) (illustrated in FIG. 1).

| Grade | Term | Description |
|---|---|---|
| 1 | Minimal | No bands, or small disjointed bands |
| 2 | Mild | Disjointed bands |
| 3 | Moderate | 1 continuous band |
| 4 | Severe | 2 continuous bands |
| 5 | Extreme | 3 or more prominent bands |

“Maximum contraction” as used in this disclosure is a term of art reflecting the fact that the patient being assessed for the severity of platysma prominence has been provided with and follows instructions which would aim to obtain maximum tension in their platysma muscles. In a preferred embodiment, the patient being assessed for the severity of platysma prominence is asked to maximally contract their platysma muscles. In some embodiments, the patient is asked to pronounce the letter “e” (for example, for at least 5 seconds) while showing the patient's lower teeth and with the patient's upper lip covering the upper teeth. It is contemplated that any similar instructions aiming to obtain maximum tension in the patient's platysma muscles may be used. In specific embodiments, the patient being assessed for the severity of platysma prominence is asked to maximally contract their platysma muscles while pronouncing the letter “e”.

“Lower neck” as used in this disclosure refers to a region of the neck that is inferior to the thyroid notch and superior to the clavicle.

“Continuous vertical neck band”, “continuous neck band” or “continuous platysma band” as used in this disclosure refers to a platysma band, or a vertical neck band visible at least at maximum contraction that extends continuously from the human patient's jawline to the lower neck (i.e., a region of the neck inferior to the thyroid notch and superior to the clavicle). It shall be understood that a patient described herein, e.g., a patient who has only one or only two continuous vertical neck band(s) on a side of the neck, may have one or more non-continuous (or disjointed) neck band(s) on the side of the neck. Often, the presence of one, two, or more continuous vertical neck bands will result in blunting of the jawline (i.e., will impact jawline definition). Jawline definition refers to the sharpness of the jawline contour. A well-defined jawline is often associated with youthful appearance and means that the jaw appears more prominent and angular, compared to a blunted jawline. In a preferred embodiment, a continuous vertical neck band as described herein is a visible vertical neck band that extends continuously from the human patient's jawline to the lower neck region and results in blunting of the jawline (i.e., impacting jawline definition).

“Disjointed band” as used in this disclosure refers to a non-continuous band, for example one that does not extend from the patient's jawline to the lower neck, as illustrated in FIG. 1. The term “small disjointed band” refers for example to a non-continuous band that is less obvious, less prominent, as illustrated in FIG. 1. Often, the presence of only disjointed bands or small disjointed bands will not result in blunting of the jawline (i.e., will have no impact to jawline definition).

“Jawline dose” or “submandibular dose” refers to a dose administered to injection site(s) (for example 4 injection sites) in the upper segment of platysma muscle, inferior and parallel to the lower mandibular border (or mandibular margin) on a first side or second side of the neck. Such injection(s) are referred to as “submandibular injection(s)” or “jawline injection(s)”. The jawline dose can also be referred to as “unilateral jawline dose”. The jawline dose is further classified as first or second jawline dose, which refers to the jawline dose administered to the first side or the second side of the human patient's neck, respectively. In some embodiments, the first jawline dose is equal to the second jawline dose. In alternative embodiments, the first jawline dose is different from the second jawline dose.

“Neck band dose” refers to a dose administered to a neck band on a first side or a second side of the neck. As the methods described herein treat no more than 2 continuous neck bands per side, the neck band dose injected along a continuous neck band is further classified as first neck band dose injected along a first continuous neck band or second neck band dose injected along a second continuous neck band. The neck band dose administered to a first side or a second side of the neck can also be referred to as “unilateral neck band dose”. In some embodiments, the first neck band dose is equal to the second neck band dose. The first neck band and the second neck band can be further specified as on the first side of the patient's neck or the second side of the patient's neck.

“Unilateral dose” refers to the total dose administered to a first side or a second side of the human patient's neck. It comprises for example the jawline dose and the neck band dose.

“Bilateral dose” refers to the total dose administered to both the first side and the second side of the human patient's neck. For example, the term “bilateral neck band dose” refers to the total neck band dose administered to both sides of the neck and the term “bilateral jawline dose” refers to the total jawline dose administered to both sides of the neck.

While botulinum toxins have been used in investigational studies to improve the appearance of the lower face and to treat platysmal bands, a number of incidents of adverse events associated with such treatments have been reported, including dysphagia, weakness of the neck flexor, and neck muscle weakness (see, e.g., Obagi and Golubets, 2017, J Drugs Dermatol 16(9):929-930; Dominguez et al., 2016, Emergency Medicine 48(12):551-556; and Witmanowski and Blochowiak, 2020, Postepy Dermatol Alergol 37(6):853-861). Treatment-related complications have been reported to include transient edema or ecchymosis (both of which have been observed to resolve within 1-2 days), hematoma formation, muscle soreness, headaches, and stinging at injection sites (Matarasso et al., 1999, Plast Reconstr Surg 103(2):645-652). In a study of more than 1500 subjects treated with BOTOX in the platysma, bruising was the most common AE, occurring in less than 20% of subjects; fewer than 10% reported transient, mild neck discomfort; 1% of subjects described neck weakness while exercising/lifting their head; and 1 case of dysphagia was reported after injection of 100 U of BOTOX (Matarasso et al., 1999, Plast Reconstr Surg 103(2):645-652). In a different study, the complication rate for botulinum toxin injections (i.e., incobotulinumtoxinA or abobotulinumtoxinA) into platysma bands was 15.2%, where the rate of ecchymosis/hematoma was 8.9%, mild dysphagia was 5.2% and neck weakness was 1.3% (Sugrue et al., 2019, Aesthet Surg J 39(2):201-206). Individual case reports have also been reported of patients experiencing severe dysphagia requiring nasogastric tube feeding for 6 weeks from cervical injection that may have involved the platysma bands (Carruthers J, Carruthers A. Practical cosmetic Botox techniques. J Cutan Med Surg. 1999; 3 Suppl 4:S49-52), or progressive dysphagia three days after injection of BOTULAX®, a botulinum toxin type A, into the platysma bands (Phothong et al., 2017, J Cosmet Dermatol 16(1):15-17). The adverse events, such as dysphagia, could be caused by, for example, diffusion of the botulinum toxin to the anatomic structures underlying the injection site(s) on the neck, such as the deeper musculature.

On the other hand, the present disclosure describes data showing that the various methods described herein provide a holistic approach that is both effective and safe for treating undesirable aesthetic effects of platysma muscle contraction using a botulinum toxin type A and in particular has a favorable benefit/risk profile for treating the aesthetic indication of platysma prominence (see, e.g., Section 7. Examples). Without wishing to be bound by any one theory, the favorable benefit/risk profile for platysma prominence of the various methods described herein may at least partly result from one or more of the following factors:

(1) Only patients having one or two continuous neck band(s) on a side of the neck are selected for treatment and only the side(s) having one or two continuous neck band(s) are treated. As such, the number of continuous neck bands injected per side of a neck is limited to no more than two, thereby reducing the total dose of botulinum toxin type A injected directly to the human patient's neck.

(2) Neck band dose(s) are only injected along continuous neck band(s), which are vertical neck band(s) that extend continuously from the human patient's jawline to the lower neck (i.e., a region of the neck inferior to the thyroid notch and superior to the clavicle), thereby minimizing the dose of botulinum toxin type A injected directly to the human patient's neck.

(3) The total dose of botulinum toxin type A injected to a side of the neck is variable and depends on the number of continuous neck band(s) present on the side of the neck, thereby allowing for a reduced unilateral and/or bilateral dose to be injected to the neck of those human patients who have a less degree of platysma prominence on the neck. This approach recognizes that there is a variation of platysma prominence presentation in the population, including without limitation those with one continuous neck band per side for both sides (bilateral Moderate), those with one continuous neck band on one side and two continuous necks on the other side (asymmetrical Moderate and Severe), and those with two continuous neck bands per side for both sides (bilateral Severe).

(4) Injection of a jawline dose of the botulinum toxin to the upper segment of the platysma muscle, inferior and parallel to lower mandibular border (or mandibular margin), (for example, a jawline dose of about 8 units of the botulinum toxin type A per side, which is preferably divided among 4 injection sites with each injection being about 2 units of the botulinum toxin type A) may generate a positive effect on the platysma muscle in the neck, thereby allowing a lower dose of the botulinum toxin type A to be injected along the neck band(s) without reducing efficacy (for example, allowing a neck band dose of about 5 units of the botulinum toxin type A per neck band, which is preferably divided among 5 injection sites with each injection being about 1 unit of the botulinum toxin type A). The jawline dose is injected close to the platysma muscle attachment (at the jawline) and may relax the entire muscle sheet so that the muscle drapes more smoothly across the jawline and down the neck.

(5) The dose of the botulinum toxin type A injected to the platysma muscle along a neck band is lower than a jawline or submandibular dose. Injection of a lower dose of botulinum toxin to the neck band reduces the risk of negatively impacting the anatomic structures underlying the injection site(s) (such as the deeper musculature) and minimizes the risk of adverse events.

(6) The neck band dose of botulinum toxin type A per injection site (e.g., 1 unit of botulinum toxin type A per injection site) is low, thereby reducing the risk of negatively impacting the anatomic structures underlying the injection site(s) (such as deeper musculature) and minimizing the risk of adverse events.

In various aspects and embodiments, a method described herein results in a reduced incidence of adverse effects as compared to a method for treating platysma prominence (e.g., for improving the appearance of platysma prominence) that does not have one, two, three, four, five, or all of the above listed factors.

The benefits of the various methods described herein for treating platysma prominence include aesthetic improvement in the appearance of the jawline in addition to aesthetic improvement in the appearance of neck bands. Without wishing to be bound by any one theory, the positive effect on the jawline may at least partly result from the inclusion of jawline injection(s) described herein in the holistic treatment approach, or the inclusion of a combination of jawline injection(s) described herein and neck band injection(s) described herein in the holistic treatment approach. Thus, treatment of platysma prominence according to the methods described herein encompasses both the treatment of blunting of the jawline and improvement of the appearance of neck bands.

Further benefits of the present disclosure will be apparent to one skilled in the art. The embodiments and aspects described in this disclosure are intended to illustrate the invention and should not be deemed to narrow the scope of the invention.

### 5.1. Definitions

As used in this disclosure, the singular forms “a”, “an” and “the” include plural referents unless the context clearly dictates otherwise. The terms “a” (or “an”), as well as the terms “one or more,” and “at least one” can be used interchangeably herein unless the context clearly dictates otherwise.

As used in this disclosure and unless otherwise specified, the term “about” or “approximately” as used herein means within an acceptable error range for the particular value as determined by one of ordinary skill in the art, which will depend in part on how the value is measured or determined (i.e., the limitations of the measurement system). For example, “about” or “approximately” can mean within 1 or more than 1 standard deviations, per practice in the art, or mean allowing a variation within 20% or 10% or 5% of the stated value. Where particular values are described in the application and claims, unless otherwise stated, the term “about” or “approximately” means within an acceptable error range for the particular value. In specific embodiments, the terms “about” and “approximately” encompass the exact value recited. Unless the context clearly dictates otherwise, all numerical values provided herein are modified by the term about.

“Administration”, or “to administer” means the step of giving (i.e. administering) a composition (e.g., a pharmaceutical composition) to a subject, or alternatively a subject receiving a composition (e.g., a pharmaceutical composition). The compositions (e.g., pharmaceutical compositions) disclosed herein can be locally administered by various methods. For example, intramuscular, intradermal, subcutaneous administration, topical (transdermal), instillation, and implantation (for example, of a slow-release device such as polymeric implant or miniosmotic pump) can all be appropriate routes of administration.

The terms “and” and “or” can be used interchangeably and can be understood to mean “and/or” unless the context clearly dictates otherwise.

“Animal” means a mammal (such as a human), bird, reptile, fish, insect, spider or other animal species. “Animal” excludes microorganisms, such as bacteria. An “animal protein free” pharmaceutical composition can include a botulinum toxin. For example, an “animal protein free” pharmaceutical composition means a pharmaceutical composition which is either substantially free or essentially free or entirely free of a serum derived albumin, gelatin and other animal derived proteins, such as immunoglobulins. An example of an animal protein free pharmaceutical composition is a pharmaceutical composition which comprises, or which consists of a botulinum toxin (as the active ingredient) and a suitable polysaccharide as a stabilizer or excipient.

“Biological activity” describes the beneficial or adverse effects of a drug on living matter. When a drug is a complex chemical mixture, this activity is exerted by the substance's active ingredient but can be modified by the other constituents. Biological activity can be assessed as potency or as toxicity by an in vivo LD₅₀ or ED₅₀ assay, or through an in vitro assay such as, for example, cell-based potency assays as described in U.S. Patent Application Publication Nos. 2010/0203559 and 2010/0233802.

“Botulinum toxin” means a neurotoxin produced by Clostridium botulinum, as well as a botulinum toxin (or the light chain or the heavy chain thereof) made recombinantly by a non-Clostridial species. The phrase “botulinum toxin”, as used herein, encompasses botulinum neurotoxin serotype A (BoNT/A) and/or subtypes and variants thereof. “Botulinum toxin”, as disclosed herein, includes, without limitation, naturally occurring botulinum toxins, fragments, or chimeras thereof, non-naturally occurring botulinum toxins, such as recombinant, modified botulinum toxins, fragments, or chimeras thereof. Further, “botulinum toxin” as used herein also encompasses a botulinum toxin complex, (for example, the 300, 500, 600 and 900 kDa complexes), as well as the neurotoxic component of the botulinum toxin (150 kDa) that is unassociated with the complex proteins. Throughout this disclosure, “botulinum toxin type A”, “botulinum toxin serotype A”, “botulinum neurotoxin type A” and “botulinum neurotoxin serotype A” are used interchangeably.

It is understood that wherever aspects and embodiments are described in this disclosure with the language “comprising,” otherwise analogous aspects described in terms of “consisting of” and/or “consisting essentially of” are also provided.

“Effective amount” as applied to the biologically active ingredient means that amount of the ingredient which is generally sufficient to effect a desired change in the subject.

“Local administration” means direct administration of a pharmaceutical at or to the vicinity of a site on or within an animal body, at which site a biological effect of the pharmaceutical is desired, such as via, for example, intramuscular or intra- or subdermal injection or topical administration. Local administration excludes systemic routes of administration, such as intravenous or oral administration. Topical administration is a type of local administration in which a pharmaceutical agent is applied to a patient's skin.

The terms “patient”, “subject” and “participant” are used herein interchangeably and refer to a human. In a specific embodiment, the subject is a human adult. In a specific embodiment, the subject is younger than age 65.

“Pharmaceutical composition” means a composition comprising an active pharmaceutical ingredient, such as, for example, a botulinum toxin, and at least one additional ingredient, such as, for example, a stabilizer or excipient or the like. A pharmaceutical composition is therefore a formulation which is suitable for diagnostic or therapeutic administration to a subject, such as a human patient. The pharmaceutical composition can be, for example, in a lyophilized or vacuum dried condition, a solution formed after reconstitution of the lyophilized or vacuum dried pharmaceutical composition, or as a solution or solid which does not require reconstitution.

The term “prominence” is used to describe a disruption to a normally smooth or contoured surface by a protruding or projecting anatomical feature, similar to laryngeal prominence. “Platysma prominence” refers to an aesthetically unappealing disruption to the lines and contour of the lower face and neck by a protruding platysma muscle at contraction. This contraction causes the muscle to bulge on the lower face, blunting the jawline, and creates vertical bands along the length of the neck.

“Improve or improving the appearance of platysma prominence” means reduce or reducing the severity of platysma prominence, which includes aesthetic improvement in the appearance of: (i) the jawline, and (ii) the vertical neck bands. Thus, improving the appearance of platysma prominence includes but is not limited to treating the jawline, improving the jawline contour, sharpening the jawline, slimming the jawline, reducing the blunting of the jawline, rejuvenating the jawline, reducing downward pull of the oral commissure, redefining the oral commissure and elevating the angle of the mouth. Improving the appearance of platysma prominence also includes improving the appearance of the platysma bands, reducing the prominence of the platysma bands, changing the neck bands to be less visible or less obvious, and improving the neck contour to become a firmer, smoother, and tighter neck. For example and without limitation, in some embodiments, improving the appearance of platysma prominence comprises an at least one grade improvement on the APPS scale (including C-APPS and P-APPS). In some embodiments, improving the appearance of platysma prominence comprises at least a change from baseline on the patient reported outcomes (PROs) such as ANLFQ and/or BAS-PP. In a specific embodiment, an improvement in appearance as described herein is a temporary improvement in appearance.

“Treating” or “treat” or “treatment” means to alleviate, improve the appearance of, or eliminate at least one symptom of a condition, such as, for example, an undesirable aesthetic effect or the like, either temporarily or permanently. Treating platysma prominence as used herein means to improve the appearance of platysma prominence, as defined above, associated with platysma muscle activity or reducing platysma prominence in a human patient or subject, either temporarily or permanently. In some embodiments, treating platysma prominence comprises minimizing or reducing the appearance of platysma bands in a patient. For example and without limitation, in some embodiments, treating platysma prominence comprises an at least one grade improvement on the APPS scale (including C-APPS and P-APPS).

“Unit” or “U” refers to the LD₅₀ dose or the dose determined by a cell-based potency assay (CBPA). The LD₅₀ dose is defined as the amount of a botulinum toxin that killed 50% of the mice injected with the botulinum toxin. The CBPA dose is determined as described in U.S. Pat. Nos. 8,618,261; 8,198,034; 9,249,216; 10,703,806; 11,261,240 and 11,332,518; the assay details of which are incorporated by reference herein.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### 5.2. Botulinum Toxins

The genus Clostridium has more than one hundred and thirty species, grouped by morphology and function. The anaerobic, gram positive bacterium Clostridium botulinum produces a potent polypeptide neurotoxin, botulinum toxin (synonymously “toxin”), which causes a neuroparalytic illness in humans and animals known as botulism. Symptoms of botulinum toxin intoxication can progress from difficulty walking, swallowing, and speaking to paralysis of the respiratory muscles and death.

Although all the botulinum toxin serotypes apparently inhibit release of the neurotransmitter acetylcholine at the neuromuscular junction, they do so by affecting different neurosecretory proteins and/or cleaving these proteins at different sites. Botulinum toxin type A is a zinc endopeptidase which can specifically hydrolyze a peptide linkage of the intracellular, vesicle-associated protein (VAMP, also called synaptobrevin) 25 kiloDalton (kDa) synaptosomal associated protein (SNAP-25). Botulinum type E also cleaves SNAP-25 but targets different amino acid sequences within this protein, as compared to botulinum toxin type A. Botulinum toxin types B, D, F and G act on VAMP with each serotype cleaving the protein at a different site. Finally, botulinum toxin type C₁ has been shown to cleave both syntaxin and SNAP-25. These differences in mechanism of action may affect the relative potency and/or duration of action of the various botulinum toxin serotypes.

The molecular weight of the active botulinum toxin protein molecule (also known as “pure toxin” or as the “neurotoxic component”) from a botulinum toxin complex, for all of the known botulinum toxin serotypes, is about 150 kDa. Interestingly, the botulinum toxins are released by Clostridial bacterium as complexes comprising the 150 kDa neurotoxic component along with one or more associated non-toxin proteins. Thus, the botulinum toxin type A complex can be produced by Clostridial bacterium as 900 kDa, 500 kDa and 300 kDa forms (approximate molecular weights). The complexes (i.e., molecular weight greater than about 150 kDa) contain hemagglutinin (HA) proteins and a non-toxin non-hemagglutinin (NTNH) protein. Thus, a botulinum toxin complex can comprise a botulinum toxin molecule (the neurotoxic component) and one or more HA proteins and/or NTNH protein. These two types of non-toxin proteins (which along with the botulinum toxin molecule can comprise the relevant neurotoxin complex) may act to provide stability against denaturation to the botulinum toxin molecule and protection against digestive acids when toxin is ingested. Additionally, it is possible that the larger (greater than about 150 kDa molecular weight) botulinum toxin complexes may result in a slower rate of diffusion of the botulinum toxin away from a site of intramuscular injection of a botulinum toxin complex.

The botulinum toxin type A is known to be soluble in dilute aqueous solutions at pH 4-6.8. At pH above about 7 the stabilizing non-toxin proteins dissociate from the neurotoxin, resulting in a gradual loss of toxicity, particularly as the pH and temperature rise (Schantz E. J., et al Preparation and characterization of botulinum toxin type A for human treatment, chapter 3 of Jankovic, J., et al, Therapy with Botulinum Toxin, Marcel Dekker, Inc, 1994).

As with enzymes generally, the biological activities of the botulinum toxins (which are intracellular peptidases) are dependent, at least in part, upon their three-dimensional conformation. Dilution of the toxin from milligram quantities to a solution containing nanograms per milliliter presents significant difficulties, such as, for example, tendency for toxin to adhere to surfaces and thus reduce the amount of available toxin. Since the toxin may be used months or years after the toxin containing pharmaceutical composition is formulated, the toxin is stabilized with a stabilizing agent or excipient. Acceptable excipients or stabilizers include protein excipients, such as albumin or gelatin, or the like, or non-protein excipients, including poloxamers, saccharides, polyethylene glycol, hyaluronic acid or the like. Use of non-protein excipients in botulinum toxin formulations is disclosed in U.S. Pat. Nos. 10,360,190 and 10,973,890; International Patent Application Publications Nos. WO2018053021, WO2018053004 and WO2020056371; each of which is hereby incorporated by reference in its entirety.

Animal protein free and/or chromatographic methods for obtaining a botulinum toxin are disclosed in U.S. Pat. Nos. 7,160,699; 7,354,740; 7,189,541; 7,445,914; 7,452,697; 7,560,251; 8,409,828; 8,008,044; 8,012,716; 8,841,110 and 9,725,705 and 7,189,541; each of which is hereby incorporated by reference in its entirety. Animal protein free processes and systems for obtaining a botulinum toxin are also disclosed in U.S. Pat. Nos. 8,129,139; 8,927,229; 8,357,541; 8,932,827; 8,324,349; 9,206,409; 9,719,076; 10,465,178; 11,124,786; 11,203,748; 11,326,155, and U.S. Patent Application Publication Nos. 2022/0145279; 2022/0228136; 2022/0204958; 2022/0251532; 2022/0298496; 2023/0021648 and 2023/0029327; each of which is hereby incorporated by reference in its entirety.

It is contemplated that any of the currently available or future commercially available botulinum toxin formulations are suitable for the methods described herein, including but not limited to onabotulinumtoxinA (known under tradenames such as BOTOX®, BOTOX® Cosmetic, or VISTABEL®), abobotulinumtoxinA (known under tradename(s) such as DYSPORT®), incobotulinumtoxinA (known under tradename(s) such as XEOMIN®), prabotulinumtoxinA (known under tradename(s) such as JEUVEAU®), daxibotulinumtoxinA (known under tradename(s) such as DAXXIFY®), letibotulinumtoxinA (known under tradename(s) such as BOTULAX®), NEURONOX®, nivobotulinumtoxinA (known under tradename(s) such as INNOTOX®), and gemibotulinumtoxinA.

In some embodiments, the botulinum toxin used by a method described herein is a botulinum toxin type A (or serotype A). The botulinum toxin can be a recombinant botulinum toxin, such as botulinum toxins produced by E. coli.

In some embodiments, the botulinum toxin type A is selected from onabotulinumtoxinA, incobotulinumtoxinA, abotulinumtoxinA, daxibotulinumtoxinA, prabotulinumtoxinA, letibotulinumtoxinA, lanbotulinumtoxinA, nivobotulinumtoxinA, gemibotulinumtoxinA and NEURONOX®. In one embodiment, the botulinum toxin type A is onabotulinumtoxinA.

In some embodiments, the botulinum toxin is a pure neurotoxin, devoid of complexing proteins. In some embodiments, the pure neurotoxin is selected from incobotulinumtoxinA and daxibotulinumtoxinA.

In some embodiments, the botulinum toxin is in an animal protein free formulation. In some embodiments, the botulinum toxin is gemibotulinumtoxinA. In an alternative embodiment, the botulinum toxin is nivobotulinumtoxinA. In another embodiment, the botulinum toxin is daxibotulinumtoxinA.

The botulinum toxin for use according to the present methods can be stored in lyophilized, vacuum dried form in containers under vacuum pressure or as stable liquids. Prior to lyophilization the botulinum toxin can be combined with pharmaceutically acceptable excipients, stabilizers and/or carriers, such as, for example, albumin, or the like. Acceptable excipients or stabilizers include protein excipients, such as albumin or gelatin, or the like, or non-protein excipients, including poloxamers, saccharides, polyethylene glycol, or the like. In embodiments containing albumin, the albumin can be, for example, human serum albumin or recombinant human albumin, or the like. The lyophilized material can be reconstituted with a suitable liquid such as, for example, saline, water, or the like to create a solution or composition containing the botulinum toxin to be administered to the subject.

The effective amount of the botulinum toxin administered according to the present method can vary according to the potency of the toxin and particular characteristics of the condition being treated, including its severity and other various subject variables including size, weight, age, and responsiveness to therapy. The potency of the toxin is expressed as a multiple of the LD₅₀ value for the mouse, where one unit (U) of toxin may be defined as the equivalent amount of toxin that kills 50% of a group of 18 to 20 female Swiss-Webster mice, weighing about 20 grams each or through a cell-based potency assay such as described in U.S. Patent Application Publication Nos. 2010/0203559 and U.S. 2010/0233802.

The effective amount of the botulinum toxin can vary according to the potency of a botulinum toxin, as commercially available botulinum toxin formulations do not have equivalent potency units. In one embodiment, the effective amount of botulinum toxin type A administered in accord with the method is about 20 units or less for one side of the patient's neck. In one embodiment, the effective amount of botulinum toxin type A administered in accord with the method is about 18 units or less for one side of the patient's neck.

In one embodiment, the effective amount of botulinum toxin administered is in the form of a composition that can be a stable liquid pharmaceutical composition, such as a stable liquid pharmaceutical composition reconstituted from a stable solid (e.g., lyophilized) pharmaceutical composition. In one embodiment, the effective amount of botulinum toxin administered is in the form of a composition that can be a solid (e.g., lyophilized) pharmaceutical composition. The composition, the liquid pharmaceutical composition, or the solid pharmaceutical composition may comprise the botulinum toxin and a pharmacologically acceptable excipient. As used herein “pharmacologically acceptable excipient” is synonymous with “pharmacological excipient” or “excipient” and refers to any excipient that has substantially no long term or permanent detrimental effect when administered to mammal and encompasses compounds such as, e.g., stabilizing agent, a bulking agent, a cryo-protectant, a lyo-protectant, an additive, a vehicle, a carrier, a diluent, or an auxiliary. An excipient generally is mixed with an active ingredient or permitted to dilute or enclose the active ingredient and can be a solid, semi-solid, or liquid agent. In an embodiment, the composition, the liquid pharmaceutical composition, or the solid pharmaceutical composition comprises the botulinum toxin, a disaccharide, a surfactant and an antioxidant. In another embodiment, the composition, the liquid pharmaceutical composition, or the solid pharmaceutical composition comprises the botulinum toxin, a disaccharide, a surfactant and an animal protein, such as an albumin. In another embodiment, the composition, the liquid pharmaceutical composition, or the solid pharmaceutical composition comprises the botulinum toxin, a disaccharide, a surfactant and does not contain an animal protein; that is, the composition, the liquid pharmaceutical composition, or the solid pharmaceutical composition is an animal protein free composition.

It is also envisioned that a pharmaceutical composition comprising a botulinum toxin active ingredient can include one or more pharmaceutically acceptable excipients that facilitate processing of an active ingredient into pharmaceutically acceptable compositions. Insofar as any pharmacologically acceptable excipient is not incompatible with the botulinum toxin active ingredient, its use in pharmaceutically acceptable compositions is contemplated. Non-limiting examples of pharmacologically acceptable excipients can be found in, e.g., Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and HANDBOOK OF PHARMACEUTICAL EXCIPIENTS (Raymond C. Rowe et al., APhA Publications, 4^{th} edition 2003), each of which is hereby incorporated by reference in its entirety.

The constituent ingredients of a pharmaceutical composition can be included in a single composition (that is, all the constituent ingredients, except for any required reconstitution fluid, are present at the time of initial compounding of the pharmaceutical composition) or as a two-component system, for example a vacuum-dried composition reconstituted with a reconstitution vehicle which can, for example, contain an ingredient not present in the initial compounding of the pharmaceutical composition. A two-component system can provide several benefits, including that of allowing incorporation of ingredients which are not sufficiently compatible for long-term shelf storage with the first component of the two-component system. For example, the reconstitution vehicle may include a preservative which provides sufficient protection against microbial growth for the use period, for example one-week of refrigerated storage, but is not present during the two-year freezer storage period during which time it might degrade the toxin. Other ingredients, which may not be compatible with a botulinum toxin or other ingredients for long periods of time, can be incorporated in this manner; that is, added in a second vehicle (e.g., in the reconstitution vehicle) at the approximate time of use.

The pharmaceutical composition can also include preservative agents such as benzyl alcohol, benzoic acid, phenol, parabens and sorbic acid. Pharmaceutical compositions can include, for example, excipients, such as surface active agents; dispersing agents; inert diluents; granulating and disintegrating agents; binding agents; lubricating agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; antioxidants; stabilizing agents; and pharmaceutically acceptable polymeric or hydrophobic materials and other ingredients known in the art and described, for example in Genaro, ed., 1985, Remington's Pharmaceutical Sciences, Mack Publishing Co., Easton, Pa., which is incorporated herein by reference.

In one embodiment, the botulinum toxin is provided in single use vials in the form of lyophilized powder. For example, the vial contains, in some embodiments, between about 10-750 units or between about 100-240 units of a botulinum toxin type A, and any excipients. Prior to use, the lyophilate can be reconstituted with, for example, sterile, non-preserved 0.9% Sodium Chloride Injection USP. In one embodiment, a vial comprises lyophilized 50 units, 100 units or 200 units of botulinum toxin for reconstitution with sterile, non-preserved 0.9% sodium chloride USP.

In other embodiments, the botulinum toxin is provided in single-use pre-filled syringes. Pre-filled syringes are pre-reconstituted with sterile, non-preserved 0.9% sodium chloride Injection USP. The pre-filled syringes can comprise between about 1-5 mL, or preferably about 3 mL, and may comprise between about 10-150 units, or between about 20-100 units, 20-60 units, or 25-50 units of a botulinum toxin type A. In one embodiment, a pre-filled syringe comprises 3 mL with 50 units or 200 units of a botulinum toxin type A.

### 5.3. Methods of Use

The present disclosure provides methods for improving the appearance of platysma prominence to a patient who has only one or only two continuous vertical neck bands on at least one side of the patient's neck. Preferably, the methods involve the injection to the side of the patient's neck of a jawline dose of the botulinum toxin type A composition below the patient's jawline and neck band dose(s) of the botulinum toxin type A composition along one or two continuous bands.

Certain methods for improving the appearance of platysma prominence as provided herein contain a step of determining the number of continuous vertical neck bands a patient has and selecting a patient accordingly for treatment. Thus, in one aspect, provided herein is a method for improving (e.g., temporarily improving) the appearance of platysma prominence (e.g., moderate to severe platysma prominence) associated with platysma muscle activity to a human patient, comprising quantifying the number of continuous vertical neck bands at maximum contraction on a first side of the human patient's neck, wherein a continuous vertical neck band is a vertical neck band that extends continuously from the human patient's jawline to a lower neck region; and then administering a composition comprising a botulinum toxin type A to the first side of the human patient's neck determined as having only one or only two continuous vertical neck bands at maximum contraction.

In specific embodiments, a method described herein for improving (e.g., temporarily improving) the appearance of platysma prominence (e.g., moderate to severe platysma prominence) associated with platysma muscle activity is for improving (e.g., temporarily improving) the appearance of platysma bands (e.g., moderate to severe platysma bands) associated with platysma muscle activity. In specific embodiments, a method described herein for improving (e.g., temporarily improving) the appearance of platysma prominence (e.g., moderate to severe platysma prominence) associated with platysma muscle activity is for improving (e.g., temporarily improving) the appearance of platysma prominence (e.g., moderate to severe platysma prominence) seen at maximum contraction. In specific embodiments, a method described herein for improving (e.g., temporarily improving) the appearance of platysma prominence (e.g., moderate to severe platysma prominence) associated with platysma muscle activity is for improving (e.g., temporarily improving) the appearance of platysma bands prominence (e.g., moderate to severe platysma bands prominence) seen at maximum contraction. In specific embodiments, a method described herein for improving (e.g., temporarily improving) the appearance of platysma prominence (e.g., moderate to severe platysma prominence) associated with platysma muscle activity is for improving (e.g., temporarily improving) the appearance of vertical bands connecting the jaw and neck (e.g., moderate to severe vertical bands connecting the jaw and neck seen at maximum contraction.

In a specific embodiment, the method provided herein for improving the appearance of platysma prominence associated with platysma muscle activity to a human patient comprises identifying a human patient with platysma prominence characterized by at least one continuous vertical neck band at maximum contraction on a side of the human patient's neck; quantifying the number of continuous vertical neck bands at maximum contraction on the side of the human patient's neck; classifying the side of the human patient's neck as (i) having moderate platysma prominence if the side of the human patient's neck has only one continuous vertical neck band at maximum contraction, or (ii) having severe platysma prominence if the side of the human patient's neck has only two continuous vertical neck bands at maximum contraction; and administering a composition comprising a botulinum toxin type A to the side of the human patient's neck if the side of the human patient is classified as having moderate or severe platysma prominence.

If a human patient has been determined as having only one continuous vertical neck band on a side of the human patient's neck, the composition is administered to the side of the human patient's neck by injection of: a jawline dose (e.g., divided among 4 injection sites) in the upper segment of platysma muscle, inferior and parallel to the patient's lower mandibular border (or mandibular margin) on the side, and the injection sites (e.g., the 4 injection sites) may comprise an anterior injection site in line with the oral commissure and a posterior injection site anterior to (e.g., slightly anterior to) the angle of the mandible; and a neck band dose (e.g., divided among 5 injection sites) along the only continuous vertical neck band.

If a human patient has been determined as having only two continuous vertical neck bands on a side of the human patient's neck, the composition is administered to the side of the human patient's neck by injection of: a jawline dose (e.g., divided among 4 injection sites) in the upper segment of platysma muscle, inferior and parallel to the patient's lower mandibular border (or mandibular margin) on the side, and the injection sites (e.g., the 4 injection sites) may comprise an anterior injection site in line with the oral commissure and a posterior injection site anterior to (e.g., slightly anterior to) the angle of the mandible; and a first neck band dose (e.g., divided among 5 injection sites) along a first continuous vertical neck band and a second neck band dose (e.g., divided among 5 injection sites) along a second continuous vertical neck band.

Whether an injection site is “slightly anterior” to the angle of the mandible may be determined by the treating physician, or based on an art-accepted standard or a standard set forth by a person of ordinary skill in the art.

The method can further comprise repeating the steps of quantifying and administering to a second side of the human patient's neck.

The present disclosure provides specific dosing schemes to a human patient determined as having only one or two continuous vertical neck band(s) on either side of the patient' neck.

For example, when the first side of the human patient's neck has only one continuous vertical neck band, the composition can be administered to the first side of the human patient's neck by injection of: (i) a first jawline dose (e.g., divided among 4 injection sites) in the upper segment of platysma muscle, inferior and parallel to the patient's lower mandibular border (or mandibular margin) on the first side, wherein optionally the injection sites (e.g., the 4 injection sites) comprise an anterior injection site in line with the oral commissure and a posterior injection site anterior to (e.g., slightly anterior to) the angle of the mandible; and (ii) a first neck band dose (e.g., divided among 5 injection sites) along the only continuous vertical neck band. In specific embodiments, the administering step delivers a total unilateral dose of about 10 units to about 15 units of botulinum toxin type A to the first side of the human patient's neck. In another specific embodiment, the administering step delivers a total unilateral dose of about 13 units of botulinum toxin type A to the first side of the human patient's neck. In a specific embodiment, the composition is administered by injection of: (i) a first jawline dose of about 8 units of botulinum toxin type A divided among 4 injection sites (e.g., with each injection being about 2 units of botulinum toxin type A) in the upper segment of platysma muscle, inferior and parallel to the patient's lower mandibular border (or mandibular margin) on the first side, the 4 injection sites comprising an anterior injection site in line with the oral commissure and a posterior injection site anterior to (e.g., slightly anterior to) the angle of the mandible; and (ii) a first neck band dose of about 5 units of botulinum toxin type A divided among 5 injection sites (e.g., with each injection being about 1 unit of botulinum toxin type A) along the only continuous vertical neck band.

In a specific embodiment wherein the human patient has only one continuous vertical neck band at maximum contraction on a first side of the neck, the method provided herein comprises administering a composition comprising a botulinum toxin type A to the first side of the human patient's neck, by injection of: (i) a first jawline dose of about 8 units of botulinum toxin type A divided among 4 injection sites in the upper segment of platysma muscle, inferior and parallel to the patient's lower mandibular border (or mandibular margin) on the first side, the 4 injection sites comprising an anterior injection site in line with the oral commissure and a posterior injection site anterior to (e.g., slightly anterior to) the angle of the mandible, with each injection of the first jawline dose being about 2 units of botulinum toxin type A; and (ii) a first neck band dose of about 5 units of botulinum toxin type A divided among 5 injection sites along the only continuous vertical neck band, with each injection of the first neck band dose being about 1 unit of botulinum toxin type A; wherein the administering step delivers a total unilateral dose of about 13 units of botulinum toxin type A to the first side of the human patient's neck.

In certain situations, when both the first side and the second side of the human patient's neck have only one continuous vertical neck band, the composition can be administered to the first side and second side of the human patient's neck by injection of: (i) a first jawline dose (e.g., divided among 4 injection sites) in the upper segment of platysma muscle, inferior and parallel to the patient's lower mandibular border (or mandibular margin) on the first side, wherein optionally the injection sites (e.g., the 4 injection sites) comprise an anterior injection site in line with the oral commissure and a posterior injection site anterior to (e.g., slightly anterior to) the angle of the mandible; (ii) a first neck band dose (e.g., divided among 5 injection sites) along the only continuous vertical neck band on the first side; (iii) a second jawline dose (e.g., divided among 4 injection sites) in the upper segment of platysma muscle, inferior and parallel to the patient's lower mandibular border (or mandibular margin) on the second side, wherein optionally the injection sites (e.g., the 4 injection sites) comprise an anterior injection site in line with the oral commissure and a posterior injection site anterior to (e.g., slightly anterior to) the angle of the mandible; and (iv) a first neck band dose (e.g., divided among 5 injection sites) along the only continuous vertical neck band on the second side. In specific embodiments, the administering step delivers a total unilateral dose of about 10 units to about 15 units of botulinum toxin type A to each side of the human patient's neck. In another specific embodiment, the administering step delivers a total bilateral dose of about 20 units to about 30 units of botulinum toxin type A to the human patient's neck. In specific embodiments, the administering step delivers a total unilateral dose of about 13 units of botulinum toxin type A to each side of the human patient's neck. In specific embodiments, the administering step delivers a total bilateral dose of about 26 units of botulinum toxin type A to the human patient's neck. In a more specific embodiment, the composition is administered by injection of: (i) a first jawline dose of about 8 units of botulinum toxin type A divided among 4 injection sites (e.g., with each injection being about 2 units of botulinum toxin type A) in the upper segment of platysma muscle, inferior and parallel to the patient's lower mandibular border (or mandibular margin) on the first side, the 4 injection sites comprising an anterior injection site in line with the oral commissure and a posterior injection site anterior to (e.g., slightly anterior to) the angle of the mandible; (ii) a first neck band dose of about 5 units of botulinum toxin type A divided among 5 injection sites (e.g., with each injection being about 1 unit of botulinum toxin type A) along the only continuous vertical neck band on the first side; (iii) a second jawline dose of about 8 units of botulinum toxin type A divided among 4 injection sites (e.g., with each injection being about 2 units of botulinum toxin type A) in the upper segment of platysma muscle, inferior and parallel to the patient's lower mandibular border (or mandibular margin) on the second side, the 4 injection sites comprising an anterior injection site in line with the oral commissure and a posterior injection site anterior to (e.g., slightly anterior to) the angle of the mandible; and (iv) a first neck band dose of about 5 units of botulinum toxin type A divided among 5 injection sites (e.g., with each injection being about 1 unit of botulinum toxin type A along the only continuous vertical neck band on the second side.

In a specific embodiment wherein the human patient has only one continuous vertical neck band at maximum contraction on a first side of the neck and only one continuous vertical neck band at maximum contraction on a second side of the neck, the method provided herein comprises: (A) administering a composition comprising a botulinum toxin type A to the first and second sides of the human patient's neck, by injection of: (i) a first jawline dose of about 8 units of botulinum toxin type A divided among 4 injection sites in the upper segment of platysma muscle, inferior and parallel to the patient's lower mandibular border (or mandibular margin) on the first side, the 4 injection sites comprising an anterior injection site in line with the oral commissure and a posterior injection site anterior to (e.g., slightly anterior to) the angle of the mandible, with each injection of the first jawline dose being about 2 units of botulinum toxin type A; and (ii) a first neck band dose of about 5 units of botulinum toxin type A divided among 5 injection sites along the only continuous vertical neck band on the first side, with each injection of the first neck band dose being about 1 unit of botulinum toxin type A; and (B) further administering the composition to the human patient's neck, by injection of: (iii) a second jawline dose of about 8 units of botulinum toxin type A divided among 4 injection sites in the upper segment of platysma muscle, inferior and parallel to the patient's lower mandibular border (or mandibular margin) on the second side, the 4 injection sites comprising an anterior injection site in line with the oral commissure and a posterior injection site anterior to (e.g., slightly anterior to) the angle of the mandible, with each injection of the second jawline dose being about 2 units of botulinum toxin type A; and (iv) a first neck band dose of about 5 units of botulinum toxin type A divided among 5 injection sites along the only continuous vertical neck band on the second side, with each injection of the first neck band dose being about 1 unit of botulinum toxin type A; wherein the step of administering to the first side and the step of administering to the second side deliver a total bilateral dose of about 26 units of botulinum toxin type A to the human patient's neck.

In yet other situations, when the first side of the human patient's neck has only two continuous vertical neck bands, the composition can be administered to the first side of the human patient's neck by injection of: (i) a first jawline dose (e.g., divided among 4 injection sites) in the upper segment of platysma muscle, inferior and parallel to the patient's lower mandibular border (or mandibular margin) on the first side, wherein optionally the injection sites (e.g., the 4 injection sites) comprise an anterior injection site in line with the oral commissure and a posterior injection site anterior to (e.g., slightly anterior to) the angle of the mandible; and (ii) a first neck band dose (e.g., divided among 5 injection sites) along a first continuous vertical neck band and a second neck band dose (e.g., divided among 5 injection sites) along a second continuous vertical neck band. In specific embodiments, the administering step delivers a total unilateral dose of about 15 units to about 20 units of botulinum toxin type A to the first side of the human patient's neck. In specific embodiments, the administering step delivers a total unilateral dose of about 18 units of botulinum toxin type A to the first side of the human patient's neck. In a specific embodiment, the composition is administered by injection of: (i) a first jawline dose of about 8 units of botulinum toxin type A divided among 4 injection sites (e.g., with each injection being about 2 units of botulinum toxin type A) in the upper segment of platysma muscle, inferior and parallel to the patient's lower mandibular border (or mandibular margin) on the first side, the 4 injection sites comprising an anterior injection site in line with the oral commissure and a posterior injection site anterior to (e.g., slightly anterior to) the angle of the mandible; and (ii) a first neck band dose of about 5 units of botulinum toxin type A divided among 5 injection sites (e.g., with each injection being about 1 unit of botulinum toxin type A) along a first continuous vertical neck band and a second neck band dose of about 5 units of botulinum toxin type A divided among 5 injection sites (e.g., with each injection being about 1 unit of botulinum toxin type A) along a second continuous vertical neck band.

In a specific embodiment wherein the human patient has only two continuous vertical neck bands at maximum contraction on a first side of the neck, the method provided herein comprises administering a composition comprising a botulinum toxin type A to the first side of the human patient's neck, by injection of: (i) a first jawline dose of about 8 units of botulinum toxin type A divided among 4 injection sites in the upper segment of platysma muscle, inferior and parallel to the patient's lower mandibular border (or mandibular margin) on the first side, the 4 injection sites comprising an anterior injection site in line with the oral commissure and a posterior injection site anterior to (e.g., slightly anterior to) the angle of the mandible, with each injection of the first jawline dose being about 2 units of botulinum toxin type A; and (ii) a first neck band dose of about 5 units of botulinum toxin type A divided among 5 injection sites along a first continuous vertical neck band and a second neck band dose of about 5 units of botulinum toxin type A divided among 5 injection sites along a second continuous vertical neck band, with each injection of the first neck band dose being about 1 unit of botulinum toxin type A and each injection of the second neck band dose being about 1 unit of botulinum toxin type A; wherein the administering step delivers a total unilateral dose of about 18 units of botulinum toxin type A to the first side of the human patient's neck.

When both the first side and the second side of the human patient's neck have only two continuous vertical neck bands, the composition can be administered to the first side and second side of the human patient's neck by injection of: (i) a first jawline dose (e.g., divided among 4 injection sites) in the upper segment of platysma muscle, inferior and parallel to the patient's lower mandibular border (or mandibular margin) on the first side, wherein optionally the injection sites (e.g., the 4 injection sites) comprise an anterior injection site in line with the oral commissure and a posterior injection site anterior to (e.g., slightly anterior to) the angle of the mandible; (ii) a first neck band dose (e.g., divided among 5 injection sites) along a first continuous vertical neck band on the first side and a second neck band dose (e.g., divided among 5 injection sites) along a second continuous vertical neck band on the first side; (iii) a second jawline dose (e.g., divided among 4 injection sites) in the upper segment of platysma muscle, inferior and parallel to the patient's lower mandibular border (or mandibular margin) on the second side, wherein optionally the injection sites (e.g., the 4 injection sites) comprise an anterior injection site in line with the oral commissure and a posterior injection site anterior to (e.g., slightly anterior to) the angle of the mandible; and (iv) a first neck band dose (e.g., divided among 5 injection sites) along a first continuous vertical neck band on the second side and a second neck band dose (e.g., divided among 5 injection sites) along a second continuous vertical neck band on the second side. In specific embodiments, the administering step delivers a total unilateral dose of about 15 units to about 20 units of botulinum toxin type A to each side of the human patient's neck. In specific embodiments, the administering step delivers a total bilateral dose of about 30 units to about 40 units of botulinum toxin type A to the human patient's neck. In specific embodiments, the administering step delivers a total unilateral dose of about 18 units of botulinum toxin type A to each side of the human patient's neck. In specific embodiments, the administering step delivers a total bilateral dose of about 36 units of botulinum toxin type A to the human patient's neck. In a specific embodiment, the composition is administered by injection of: (i) a first jawline dose of about 8 units of botulinum toxin type A divided among 4 injection sites (e.g., with each injection being about 2 units of botulinum toxin type A) in the upper segment of platysma muscle, inferior and parallel to the patient's lower mandibular border (or mandibular margin) on the first side, the 4 injection sites comprising an anterior injection site in line with the oral commissure and a posterior injection site anterior to (e.g., slightly anterior to) the angle of the mandible; (ii) a first neck band dose of about 5 units of botulinum toxin type A divided among 5 injection sites (e.g., with each injection being about 1 unit of botulinum toxin type A) along a first continuous vertical neck band on the first side and a second neck band dose of about 5 units of botulinum toxin type A divided among 5 injection sites (e.g., with each injection being about 1 unit of botulinum toxin type A) along a second continuous vertical neck band on the first side; (iii) a second jawline dose of about 8 units of botulinum toxin type A divided among 4 injection sites (e.g., with each injection being about 2 units of botulinum toxin type A) in the upper segment of platysma muscle, inferior and parallel to the lower mandibular border (or mandibular margin) on the second side, the 4 injection sites comprising an anterior injection site in line with the oral commissure and a posterior injection site anterior to (e.g., slightly anterior to) the angle of the mandible; and (iv) a first neck band dose of about 5 units of botulinum toxin type A divided among 5 injection sites (e.g., with each injection being about 1 unit of botulinum toxin type A) along a first continuous vertical neck band on the second side and a second neck band dose of about 5 units of botulinum toxin type A divided among 5 injection sites (e.g., with each injection being about 1 unit of botulinum toxin type A) along a second continuous vertical neck band on the second side.

In a specific embodiment wherein the human patient has only two continuous vertical neck bands at maximum contraction on a first side of the neck and only two continuous vertical neck bands at maximum contraction on a second side of the neck, the method provided herein comprises: (A) administering a composition comprising a botulinum toxin type A to the first side of the human patient's neck, by injection of: (i) a first jawline dose of about 8 units of botulinum toxin type A divided among 4 injection sites in the upper segment of platysma muscle, inferior and parallel to the patient's lower mandibular border (or mandibular margin) on the first side, the 4 injection sites comprising an anterior injection site in line with the oral commissure and a posterior injection site anterior to (e.g., slightly anterior to) the angle of the mandible, with each injection of the first jawline dose being about 2 units of botulinum toxin type A; and (ii) a first neck band dose of about 5 units of botulinum toxin type A divided among 5 injection sites along a first continuous vertical neck band on the first side and a second neck band dose of about 5 units of botulinum toxin type A divided among 5 injection sites along a second continuous vertical neck band on the first side, with each injection of the first neck band dose being about 1 unit of botulinum toxin type A and each injection of the second neck band dose being about 1 unit of botulinum toxin type A; and (B) further administering the composition to the second side of the human patient's neck, by injection of: (iii) a second jawline dose of about 8 units of botulinum toxin type A divided among 4 injection sites in the upper segment of platysma muscle, inferior and parallel to the patient's lower mandibular border (or mandibular margin) on the second side, the 4 injection sites comprising an anterior injection site in line with the oral commissure and a posterior injection site anterior to (e.g., slightly anterior to) the angle of the mandible, with each injection of the second jawline dose being about 2 units of botulinum toxin type A; and (iv) a first neck band dose of about 5 units of botulinum toxin type A divided among 5 injection sites along a first continuous vertical neck band on the second side and a second neck band dose of about 5 units of botulinum toxin type A divided among 5 injection sites along a second continuous vertical neck band on the second side, with each injection of the first neck band dose being about 1 unit of botulinum toxin type A and with each injection of the second neck band dose being about 1 unit of botulinum toxin type A; wherein the step of administering to the first side and the step of administering to the second side deliver a total bilateral dose of about 36 units of botulinum toxin type A to the human patient's neck.

When the first side of the human patient's neck has only one continuous vertical neck band and the second side of the human patient's neck has only two continuous vertical neck bands, the composition can be administered to the first side and second side of the human patient's neck by injection of: (i) a first jawline dose (e.g., divided among 4 injection sites) in the upper segment of platysma muscle, inferior and parallel to the patient's lower mandibular border (or mandibular margin) on the first side, wherein optionally the injection sites (e.g., the 4 injection sites) comprise an anterior injection site in line with the oral commissure and a posterior injection site anterior to (e.g., slightly anterior to) the angle of the mandible; (ii) a first neck band dose (e.g., divided among 5 injection sites) along the only continuous vertical neck band on the first side; (iii) a second jawline dose (e.g., divided among 4 injection sites) in the upper segment of platysma muscle, inferior and parallel to the patient's lower mandibular border (or mandibular margin) on the second side, wherein optionally the injection sites (e.g., the 4 injection sites) comprise an anterior injection site in line with the oral commissure and a posterior injection site anterior to (e.g., slightly anterior to) the angle of the mandible; and (iv) a first neck band dose (e.g., divided among 5 injection sites) along a first continuous vertical neck band on the second side and a second neck band dose (e.g., divided among 5 injection sites) along a second continuous vertical neck band on the second side. In specific embodiments, the administering step delivers a total unilateral dose of about 10 units to about 15 units of botulinum toxin type A to the first side of the human patient's neck and about 15 units to about 20 units of botulinum toxin type A to the second side of the human patient's neck. In specific embodiments, the administering step delivers a total bilateral dose of about 25 units to about 35 units of botulinum toxin type A to the human patient's neck. In specific embodiments, the administering step delivers a total unilateral dose of about 13 units of botulinum toxin type A to the first side of the human patient's neck and about 18 units of botulinum toxin type A to the second side of the human patient's neck. In specific embodiments, the administering step delivers a total bilateral dose of about 31 units of botulinum toxin type A to the human patient's neck. In a specific embodiment, the composition is by injection of: (i) a first jawline dose of about 8 units of botulinum toxin type A divided among 4 injection sites (e.g., with each injection being about 2 units of botulinum toxin type A) in the upper segment of platysma muscle, inferior and parallel to the patient's lower mandibular border (or mandibular margin) on the first side, the 4 injection sites comprising an anterior injection site in line with the oral commissure and a posterior injection site anterior to (e.g., slightly anterior to) the angle of the mandible; (ii) a first neck band dose of about 5 units of botulinum toxin type A divided among 5 injection sites (e.g., with each injection being about 1 unit of botulinum toxin type A) along the only continuous vertical neck band on the first side; (iii) a second jawline dose of about 8 units of botulinum toxin type A divided among 4 injection sites (e.g., with each injection being about 2 units of botulinum toxin type A) in the upper segment of platysma muscle, inferior and parallel to the patient's lower mandibular border (or mandibular margin) on the second side, the 4 injection sites comprising an anterior injection site in line with the oral commissure and a posterior injection site anterior to (e.g., slightly anterior to) the angle of the mandible; and (iv) a first neck band dose of about 5 units of botulinum toxin type A divided among 5 injection sites (e.g., with each injection being about 1 unit of botulinum toxin type A) along a first continuous vertical neck band on the second side and a second neck band dose of about 5 units of botulinum toxin type A divided among 5 injection sites (e.g., with each injection being about 1 unit of botulinum toxin type A) along a second continuous vertical neck band on the second side.

In a specific embodiment wherein the human patient has only one continuous vertical neck band at maximum contraction on a first side of the neck and only two continuous vertical neck bands at maximum contraction on a second side of the neck, the method provided herein comprises administering a composition comprising a botulinum toxin type A to the human patient's neck, by injection of: (i) a first jawline dose of about 8 units of botulinum toxin type A divided among 4 injection sites in the upper segment of platysma muscle, inferior and parallel to the patient's lower mandibular border (or mandibular margin) on the first side, the 4 injection sites comprising an anterior injection site in line with the oral commissure and a posterior injection site anterior to (e.g., slightly anterior to) the angle of the mandible, with each injection of the first jawline dose being about 2 units of botulinum toxin type A; (ii) a first neck band dose of about 5 units of botulinum toxin type A divided among 5 injection sites along the only continuous vertical neck band on the first side, with each injection of the first neck band dose being about 1 unit of botulinum toxin type A; (iii) a second jawline dose of about 8 units of botulinum toxin type A divided among 4 injection sites in the upper segment of platysma muscle, inferior and parallel to the patient's lower mandibular border (or mandibular margin) on the second side, the 4 injection sites comprising an anterior injection site in line with the oral commissure and a posterior injection site anterior to (e.g., slightly anterior to) the angle of the mandible, with each injection of the second jawline dose being about 2 units of botulinum toxin type A; and (iv) a first neck band dose of about 5 units of botulinum toxin type A divided among 5 injection sites along a first continuous vertical neck band on the second side and a second neck band dose of about 5 units of botulinum toxin type A divided among 5 injection sites along a second continuous vertical neck band on the second side, with each injection of the first neck band dose being about 1 unit of botulinum toxin type A and with each injection of the second neck band dose being about 1 unit of botulinum toxin type A; wherein the administering step delivers a total bilateral dose of about 31 units of botulinum toxin type A to the human patient's neck.

The methods for improving the appearance of platysma prominence as provided herein may be applied to a patient who has already been selected on the basis of having only one or only two continuous vertical neck bands on one or both sides of the patient's neck or is otherwise known for having only one or only two continuous vertical neck bands on one or both sides of the patient's neck.

It shall be understood that the words “first” and “second” are used in this disclosure for purposes of distinguishing different sides or different doses and do not imply the sequence in which two items or actions occur unless the context clearly dictates otherwise. As such, for example, if a first neck band dose is injected along the only continuous vertical neck band present on a side of a human patient's neck, no second neck band dose is injected on this side in preferred embodiments.

In various aspects and embodiments, a jawline dose, for example the first jawline dose on the first side or the second jawline dose on the second side, is about 5 units to about 20 units of botulinum toxin type A. In a preferred embodiment, the jawline dose is about 8 units of botulinum toxin type A. In a specific embodiment, the jawline dose is about 5 units of botulinum toxin type A. In a specific embodiment, the jawline dose is about 6 units of botulinum toxin type A. In a specific embodiment, the jawline dose is about 7 units of botulinum toxin type A. In a specific embodiment, the jawline dose is about 9 units of botulinum toxin type A. In a specific embodiment, the jawline dose is about 10 units of botulinum toxin type A.

In various aspects and embodiments, the jawline dose is injected at one injection site. In various aspects and embodiments, the jawline dose is divided among more than one injection site. In specific embodiments, the jawline dose is divided among 2 to 6 injection sites. In a preferred embodiment, the jawline dose is divided among 4 injection sites. In a specific embodiment, the jawline dose is divided among 2 injection sites. In a specific embodiment, the jawline dose is divided among 3 injection sites. In a specific embodiment, the jawline dose is divided among 5 injection sites. In a specific embodiment, the jawline dose is divided among 6 injection sites. In specific embodiments, the injection sites are placed equidistant from each other. In specific embodiments, the injection sites are placed approximately 1-2 cm apart from each other.

In various aspects and embodiments, the jawline dose is divided equally among the injection sites. In alternative embodiments, the jawline dose is divided unequally among the injection sites.

In various aspects and embodiments, the jawline dose per injection site is about 1 unit to about 10 units of botulinum toxin type A. In a preferred embodiment, the jawline dose per injection site is about 2 units of botulinum toxin type A. In a specific embodiment, the jawline dose per injection site is about 1 unit of botulinum toxin type A. In a specific embodiment, the jawline dose per injection site is about 1.5 units of botulinum toxin type A. In a specific embodiment, the jawline dose per injection site is about 2.5 units of botulinum toxin type A. In a specific embodiment, the jawline dose per injection site is about 3 units of botulinum toxin type A. In a specific embodiment, the jawline dose per injection site is about 3.5 units of botulinum toxin type A. In a specific embodiment, the jawline dose per injection site is about 4 units of botulinum toxin type A. In some embodiments, the first jawline dose is equal to the second jawline dose. In alternative embodiments, the first jawline dose is different than the second jawline dose.

In various aspects and embodiments, the jawline dose is injected in the upper segment of the platysma muscle, inferior and parallel to the patient's lower mandibular border (or mandibular margin) on the first or second side of the neck. In various aspects and embodiments, the jawline dose is injected into 4 injection sites to the upper platysma muscle, approximately 1-5 cm inferior and parallel to the lower mandibular border (or mandibular margin). In specific embodiments, the jawline dose is injected into 4 injection sites to the upper platysma muscle, approximately 1-2 cm inferior and parallel to the lower mandibular border (or mandibular margin). In some embodiments, the anterior injection site of the 4 injection sites is in line with the oral commissure and the posterior injection site is anterior to (e.g., slightly anterior to) the angle of mandible. In some embodiments, the remaining 2 jawline injections are equidistant between the anterior and posterior injection points. In some embodiments, the remaining 2 jawline injections are approximately 1-2 cm apart from each other.

The jawline dose can be administered by any route suitable for injecting the composition of botulinum toxin type A to the injection site. In a preferred embodiment, the jawline dose is administered by intramuscular injection (e.g., superficial intramuscular injection). In a specific embodiment, the jawline dose is injected by intradermal injection. In a specific embodiment, the jawline dose is injected by subcutaneous injection. In some embodiments, injections to the platysma muscles are administered superficially and intramuscularly. In specific embodiments, the injections are not made deeper than about 0.5 inches into a muscle. In one embodiment, the intramuscular and superficial injections are administered with the needle perpendicular to the surface of the skin. In some embodiments, the injections are not administered above the lower mandibular border (or mandibular margin) and/or into structures deep to the platysma muscle. In some embodiments, the dose is administered using a 0.5-inch needle. In some embodiments, the dose is administered using a 1-inch needle. In specific embodiments, the dose is administered using a sterile 30-gauge syringe with a 0.5-inch needle. In specific embodiments, the dose is administered using a sterile 30-gauge syringe with a 1-inch needle. In some embodiments, the injection is guided by ultrasound or alternative imaging devices. In preferred embodiments, the injections are at least 1 cm inferior to the lower mandibular border (or mandibular margin). In preferred embodiments, the injections are not made into structures deep to the platysma muscle, particularly in the anterior region of the neck.

In various aspects and embodiments, the neck band dose, for example the first neck band dose or the second neck band dose on the first side or the second side of the patient's neck, is about 2 units to about 8 units of botulinum toxin type A. In a preferred embodiment, the neck band dose is about 5 units of botulinum toxin type A. In a specific embodiment, the neck band dose is about 2 units of botulinum toxin type A. In a specific embodiment, the neck band dose is about 3 units of botulinum toxin type A. In a specific embodiment, the neck band dose is about 4 units of botulinum toxin type A. In a specific embodiment, the neck band dose is about 6 units of botulinum toxin type A. In a specific embodiment, the neck band dose is about 7 units of botulinum toxin type A. In a specific embodiment, the neck band dose is about 8 units of botulinum toxin type A.

In various aspects and embodiments, the neck band dose is injected at one injection site. In various aspects and embodiments, the neck band dose is divided among more than one injection site. In specific embodiments, the neck band dose is divided among 2 to 7 injection sites. In a preferred embodiment, the neck band dose is divided among 5 injection sites. In a specific embodiment, the neck band dose is divided among 2 injection sites. In a specific embodiment, the neck band dose is divided among 3 injection sites. In a specific embodiment, the neck band dose is divided among 4 injection sites. In a specific embodiment, the neck band dose is divided among 6 injection sites. In a specific embodiment, the neck band dose is divided among 7 injection sites. In specific embodiments, the injection sites are placed approximately 1-2 cm apart from each other. In some embodiments, the first neck band dose and the second neck band dose both on the same side of the patient's neck are equal. In alternative embodiments, the first neck band dose and the second neck band dose both on the same side of the neck are different. In some embodiments, the first or second neck band dose on one side of the neck is equal to the first or second neck band dose on the other side of the neck. In alternative embodiments, the first or second neck band dose on one side of the neck is not equal to the first or second neck band dose on the other side of the neck.

In various aspects and embodiments, the neck band dose is divided equally among the injection sites. In alternative embodiments, the neck band dose is divided unequally among the injection sites.

In various aspects and embodiments, the neck band dose per injection site is about 0.5 unit to about 2 units of botulinum toxin type A. In a preferred embodiment, the neck band dose per injection site is about 1 unit of botulinum toxin type A. In a specific embodiment, the neck band dose per injection site is about 0.5 unit of botulinum toxin type A. In a specific embodiment, the neck band dose per injection site is about 0.75 unit of botulinum toxin type A. In a specific embodiment, the neck band dose per injection site is about 1.5 units of botulinum toxin type A. In a specific embodiment, the neck band dose per injection site is about 1.25 unit of botulinum toxin type A. In a specific embodiment, the neck band dose per injection site is about 2 units of botulinum toxin type A.

Preferably, the neck band dose injected along a neck band is lower than the jawline dose injected to the same side of the patient's neck. Preferably, the neck band dose per injection site is lower than the jawline dose per injection site for the same side of the patient's neck.

In various aspects and embodiments, the most superior injection site for the neck band dose is approximately 1-2 cm inferior to the jawline injections on the same side.

The neck band dose can be administered by any route suitable for injecting the composition of botulinum toxin type A to the injection site. In a preferred embodiment, the neck band dose is administered by intramuscular injection (e.g., superficial intramuscular injection). In a specific embodiment, the neck band dose is injected by intradermal injection. In a specific embodiment, the neck band dose is injected by subcutaneous injection. In some embodiments, injections to the platysma muscles are administered superficially and intramuscularly. In specific embodiments, the injections are not made deeper than about 0.5 inches into a muscle. In one embodiment, the intramuscular and superficial injections are administered with the needle perpendicular to the surface of the skin. In some embodiments, the injections are not administered above the lower mandibular border (or mandibular margin) and/or into structures deep to the platysma muscle. In some embodiments, the dose is administered using a 0.5-inch needle. In some embodiments, the dose is administered using a 1-inch needle. In specific embodiments, the dose is administered using a sterile 30-gauge syringe with a 0.5-inch needle. In specific embodiments, the dose is administered using a sterile 30-gauge syringe with a 1-inch needle. In some embodiments, the injection is guided by ultrasound or alternative imaging devices. In some embodiments, for neck band injections, each band is identified while the patient is contracting their platysma, and gently pinched to isolate the muscle from nearby anatomical structures during administration. In preferred embodiments, the injections are at least 1 cm inferior to the lower mandibular border (or mandibular margin). In preferred embodiments, the injections are not made into structures deep to the platysma muscle, particularly in the anterior region of the neck.

In various aspects and embodiments wherein a jawline dose and a neck band dose on a first side or a second side of the neck are administered, the jawline dose is injected before the neck band dose. In alternative embodiments, the jawline dose is administered after the neck band dose. In embodiments wherein there are two neck bands on the first side or the second side, the jawline dose can be administered first, followed by administration of the neck band doses. In alternative embodiments, the neck band doses are administered before the jawline dose. In alternative embodiments, the jawline dose is administered between the first neck band dose and the second neck band dose. In embodiments wherein a jawline dose on a first side, a jawline dose on a second side, a first neck band dose on a first side, and a first neck band dose on a second side of the neck are administered, the jawline dose on the first side and the second side is administered before the first neck band on the first side and the first neck band on the second side. In some embodiments, the sequence of administration is as follows: the jawline dose on the first side, the jawline dose on the second side, the first neck band dose on the first side, and the first neck band dose on the second side. In alternative embodiments, the sequence is as follows: the jawline dose on the first side, the first neck band dose on the first side, the jawline dose on the second side, and the first neck band on the second side.

In various aspects and embodiments wherein a jawline dose, a first neck band dose and/or a second neck band dose on a first side, a second side of the neck, or both sides are administered, the jawline dose, the first neck band dose, and/or the second neck band dose are administered within 24 hours of each other. In specific embodiments, the jawline dose, the first neck band dose and/or the second neck band dose are administered within 12 hours of each other. In specific embodiments, the jawline dose, the first neck band dose and/or the second neck band dose are administered within 6 hours of each other. In specific embodiments, the jawline dose, the first neck band dose and/or the second neck band dose are administered within 4 hours of each other. In specific embodiments, the jawline dose, the first neck band dose and/or the second neck band dose are administered within 2 hours of each other. In specific embodiments, the jawline dose, the first neck band dose and/or the second neck band dose are administered within 1 hour of each other. In specific embodiments, the jawline dose, the first neck band dose and/or the second neck band dose are administered within 0.5 hour of each other. In specific embodiments, the jawline dose, the neck band dose and/or the second neck band dose are administered within 20 minutes of each other. In specific embodiments, the jawline dose, the first neck band dose and/or the second neck band dose are administered within 10 minutes of each other. In specific embodiments, the jawline dose, the first neck band dose and/or the second neck band dose are administered within 5 minutes of each other.

In various aspects and embodiments, a method or administering step(s) described herein deliver a total unilateral dose of about 10 units to about 40 units of botulinum toxin type A to a first side or a second side of the human patient's neck. In specific embodiments, the method or administering step(s) deliver a total unilateral dose of about 10 units to about 20 units of botulinum toxin type A. In specific embodiments, the method or administering step(s) deliver a total unilateral dose of about 10 units to about 15 units of botulinum toxin type A. In a specific embodiment, the method or administering step(s) deliver a total unilateral dose of about 13 units of botulinum toxin type A. In a specific embodiment, the method or administering step(s) deliver a total unilateral dose of about 18 units of botulinum toxin type A. In a specific embodiment, the method or administering step(s) deliver a total unilateral dose of about 10 units of botulinum toxin type A. In a specific embodiment, the method or administering step(s) deliver a total unilateral dose of about 11 units of botulinum toxin type A. In a specific embodiment, the method or administering step(s) deliver a total unilateral dose of about 12 units of botulinum toxin type A. In a specific embodiment, the method or administering step(s) deliver a total unilateral dose of about 14 units of botulinum toxin type A. In a specific embodiment, the method or administering step(s) deliver a total unilateral dose of about 15 units of botulinum toxin type A. In a specific embodiment, the method or administering step(s) deliver a total unilateral dose of about 16 units of botulinum toxin type A. In a specific embodiment, the method or administering step(s) deliver a total unilateral dose of about 17 units of botulinum toxin type A. In a specific embodiment, the method or administering step(s) deliver a total unilateral dose of about 19 units of botulinum toxin type A. In a specific embodiment, the method or administering step(s) deliver a total unilateral dose of about 20 units of botulinum toxin type A.

In various aspects and embodiments, a method or administering step(s) described herein deliver a total bilateral dose of about 20 units to about 80 units of botulinum toxin type A to both sides of the human patient's neck. In specific embodiments, the method or administering step(s) deliver a total bilateral dose of about 20 units to about 40 units of botulinum toxin type A to both sides of the human patient's neck. In specific embodiments, the method or administering step(s) deliver a total bilateral dose of about 20 units to about 30 units of botulinum toxin type A to both sides of the human patient's neck. In specific embodiments, the method or administering step(s) deliver a total bilateral dose of about 30 units to about 40 units of botulinum toxin type A to both sides of the human patient's neck. In specific embodiments, the method or administering step(s) deliver a total bilateral dose of about 25 units to about 35 units of botulinum toxin type A to both sides of the human patient's neck. In a specific embodiment, the method or administering step(s) deliver a total bilateral dose of about 26 units of botulinum toxin type A to both sides of the human patient's neck. In a specific embodiment, the method or administering step(s) deliver a total bilateral dose of about 31 units of botulinum toxin type A to both sides of the human patient's neck. In a specific embodiment, the method or administering step(s) deliver a total bilateral dose of about 36 units of botulinum toxin type A to both sides of the human patient's neck.

In various aspects and embodiments of a method described herein, the platysma prominence severity grade, including the number of continuous vertical neck bands on a side of a human patient's neck is determined by a physician (e.g., the human patient's treating physician) or investigator. In other embodiments, the platysma prominence severity grade, including the number of continuous vertical neck bands on a side of a human patient's neck is determined by the human patient, subject or participant. In some embodiments, the platysma prominence severity grade, including the number of continuous vertical neck bands on a side of a human patient's neck is determined by both a physician and the human patient, subject or participant.

In various aspects and embodiments, a cosmetic effect of a method described herein is obtained within 14 days (including at 14 days) following the administration of the botulinum toxin type A, wherein the cosmetic effect comprises a physician-assessed and/or human patient-assessed platysma prominence scale (e.g., Allergan Platysma Prominence Scale) grade change from moderate or severe prior to the administration of the botulinum toxin type A to minimal or mild following the administration of the botulinum toxin type A. In some embodiments, the platysma prominence scale (e.g., Allergan Platysma Prominence Scale) grade change is assessed by a physician. In some embodiments, the platysma prominence scale (e.g., Allergan Platysma Prominence Scale) grade change is assessed by the human patient. In some embodiments, the platysma prominence scale (e.g., Allergan Platysma Prominence Scale) grade change is assessed by both a physician and the human patient. In some embodiments wherein both sides of a human patient's neck are treated with a method described herein, the cosmetic effect comprises achieving the foregoing effect on both sides of the human patient's neck. In other embodiments wherein both sides of a human patient's neck are treated with a method described herein, the cosmetic effect comprises achieving the foregoing effect on at least one side of the human patient's neck.

In various aspects and embodiments, a cosmetic effect of a method described herein is obtained within 14 days (including at 14 days) following the administration of the botulinum toxin type A, wherein the cosmetic effect comprises a physician-assessed and/or human patient-assessed 2-grade or better improvement on a platysma prominence scale (e.g., the Allergan Platysma Prominence Scale) from moderate or severe prior to the administration of the botulinum toxin type A to minimal or mild following the administration of the botulinum toxin type A. In some embodiments, the 2-grade or better improvement is assessed by a physician. In some embodiments, the 2-grade or better improvement is assessed by the human patient. In some embodiments, the 2-grade or better improvement is assessed by both a physician and the human patient. In some embodiments wherein both sides of a human patient's neck are treated with a method described herein, the cosmetic effect comprises achieving the foregoing effect on both sides of the human patient's neck. In other embodiments wherein both sides of a human patient's neck are treated with a method described herein, the cosmetic effect comprises achieving the foregoing effect on at least one side of the human patient's neck.

In various aspects and embodiments, a cosmetic effect of a method described herein is obtained within 14 days (including at 14 days) following the administration of the botulinum toxin type A, wherein the cosmetic effect comprises a physician-assessed and/or human patient-assessed 1-grade or better improvement on a platysma prominence scale (e.g., the Allergan Platysma Prominence Scale) from moderate or severe prior to the administration of the botulinum toxin type A to mild or moderate following the administration of the botulinum toxin type A. In some embodiments, the 1-grade or better improvement is assessed by a physician. In some embodiments, the 1-grade or better improvement is assessed by the human patient. In some embodiments, the 1-grade or better improvement is assessed by both a physician and the human patient. In some embodiments wherein both sides of a human patient's neck are treated with a method described herein, the cosmetic effect comprises achieving the foregoing effect on both sides of the human patient's neck. In other embodiments wherein both sides of a human patient's neck are treated with a method described herein, the cosmetic effect comprises achieving the foregoing effect on at least one side of the human patient's neck.

In various aspects and embodiments, a cosmetic effect of a method described herein is obtained within 14 days (including at 14 days) following the administration of the botulinum toxin type A, wherein the cosmetic effect comprises: (1) a physician-assessed and/or human patient-assessed platysma prominence scale (e.g., Allergan Platysma Prominence Scale) grade change from moderate or severe prior to the administration of the botulinum toxin type A to minimal or mild following the administration of the botulinum toxin type A; and (2) a physician-assessed and/or human patient-assessed 2-grade or better improvement on a platysma prominence scale (e.g., the Allergan Platysma Prominence Scale) from moderate or severe prior to the administration of the botulinum toxin type A to minimal or mild following the administration of the botulinum toxin type A. In some embodiments, the platysma prominence scale (e.g., Allergan Platysma Prominence Scale) grade change is assessed by a physician. In some embodiments, the platysma prominence scale (e.g., Allergan Platysma Prominence Scale) grade change is assessed by the human patient. In some embodiments, the platysma prominence scale (e.g., Allergan Platysma Prominence Scale) grade change is assessed by both a physician and the human patient. In some embodiments, the 2-grade or better improvement is assessed by a physician. In some embodiments, the 2-grade or better improvement is assessed by the human patient. In some embodiments, the 2-grade or better improvement is assessed by both a physician and the human patient. In some embodiments, the platysma prominence scale (e.g., Allergan Platysma Prominence Scale) grade change and the 2-grade or better improvement are assessed by a physician. In some embodiments, the platysma prominence scale (e.g., Allergan Platysma Prominence Scale) grade change and the 2-grade or better improvement are assessed by the human patient. In some embodiments, the platysma prominence scale (e.g., Allergan Platysma Prominence Scale) grade change and the 2-grade or better improvement are assessed by both a physician and the human patient. In some embodiments wherein both sides of a human patient's neck are treated with a method described herein, the cosmetic effect comprises achieving the foregoing effect on both sides of the human patient's neck. In other embodiments wherein both sides of a human patient's neck are treated with a method described herein, the cosmetic effect comprises achieving the foregoing effect on at least one side of the human patient's neck.

In various aspects and embodiments, a cosmetic effect of a method described herein is obtained within 14 days (including at 14 days) following the administration of the botulinum toxin type A, wherein the cosmetic effect comprises achieving a physician-assessed and/or human patient-assessed grade of minimal or mild and at least a 2-grade improvement from baseline on a platysma prominence scale (e.g., Allergan Platysma Prominence Scale). The baseline is assessed prior to the administration of the botulinum toxin type A. In specific embodiments, the baseline is assessed on the day of but prior to the administration of the botulinum toxin type A. In specific embodiments, the baseline is a grade of moderate or severe on the platysma prominence scale (e.g., Allergan Platysma Prominence Scale). In some embodiments, the achievement of a grade of minimal or mild and the at least 2-grade improvement is assessed by a physician. In some embodiments, the achievement of a grade of minimal or mild and the at least 2-grade improvement is assessed by the human patient. In some embodiments, the achievement of a grade of minimal or mild and the at least 2-grade improvement is assessed by both a physician and the human patient. In some embodiments wherein both sides of a human patient's neck are treated with a method described herein, the cosmetic effect comprises achieving the foregoing effect on both sides of the human patient's neck. In other embodiments wherein both sides of a human patient's neck are treated with a method described herein, the cosmetic effect comprises achieving the foregoing effect on at least one side of the human patient's neck.

The Allergan Platysma Prominence Scale as described herein in this disclosure is set forth in Table 1 above and illustrated in FIG. 1.

In certain embodiments, the cosmetic effect is obtained within 13 days, within 12 days, within 11 days, within 10 days, within 9 days, within 8 days, within 7 days, within 6 days, within 5 days, within 4 days, within 3 days, within 2 days, or within 1 day following the administration of the botulinum toxin type A.

In certain embodiments, the duration of the cosmetic effect ranges from about 60 days to about 90 days following the administration of the botulinum toxin type A. In certain embodiments, the duration of the cosmetic effect ranges from about 60 days to about 120 days following the administration of the botulinum toxin type A. In certain embodiments, the duration of the cosmetic effect ranges from about 90 days to about 120 days following the administration of the botulinum toxin type A. In certain embodiments, the duration of the cosmetic effect is at least 120 days. In certain embodiments, the duration of the cosmetic effect is at least 60 days. In certain embodiments, the duration of the cosmetic effect is at least 30 days. In certain embodiments, the duration of the cosmetic effect is at least 90 days. In certain embodiments, the duration of the cosmetic effect is assessed at a population level based on whether the percentage of human patients treated with the method who have obtained and maintained the cosmetic effect is above a set threshold (e.g., 5%, 10%, 15%, or 20%).

In certain embodiments, a method described herein is better (preferably statistically better) than a comparable method wherein a placebo is administered instead of the botulinum toxin type A at achieving a physician-assessed and/or human patient-assessed grade of minimal or mild on a platysma prominence scale (e.g., the Allergan Platysma Prominence Scale) at 14 days, 30 days, 60 days, 90 days or 120 days post-administration. In some embodiments, the grade on the platysma prominence scale is assessed by a physician. In some embodiments, the grade on the platysma prominence scale is assessed by the human patient. In some embodiments, the grade on the platysma prominence scale is assessed by both a physician and the human patient.

In certain embodiments, a method described herein is better (preferably statistically better) than a comparable method wherein a placebo is administered instead of the botulinum toxin type A at achieving a physician-assessed and/or human patient-assessed platysma prominence scale (e.g., Allergan Platysma Prominence Scale) grade change from moderate or severe prior to the administration of the botulinum toxin type A or placebo to minimal or mild at 14 days, 30 days, 60 days, 90 days or 120 days post-administration. In some embodiments, the grade change is assessed by a physician. In some embodiments, the grade change is assessed by the human patient. In some embodiments, the grade change is assessed by both a physician and the human patient.

In certain embodiments, a method described herein is better (preferably statistically better) than a comparable method wherein a placebo is administered instead of a botulinum toxin type A at achieving a physician-assessed and/or patient-assessed 1-grade or better improvement on a platysma prominence scale (e.g., the Allergan Platysma Prominence Scale) from moderate or severe prior to the administration of the botulinum toxin type A or placebo to mild or moderate at 14 days, 30 days, 60 days, 90 days or 120 days post-administration. In some embodiments, the 1-grade or better improvement is assessed by a physician. In some embodiments, the 1-grade or better improvement is assessed by the human patient. In some embodiments, the 1-grade or better improvement is assessed by both a physician and the human patient.

In certain embodiments, a method described herein is better (preferably statistically better) than a comparable method wherein a placebo is administered instead of a botulinum toxin type A at achieving a physician-assessed and/or human patient-assessed 2-grade or better improvement on a platysma prominence scale (e.g., the Allergan Platysma Prominence Scale) from moderate or severe prior to the administration of the botulinum toxin type A or placebo to minimal or mild at 14 days, 30 days, 60 days, 90 days or 120 days post-administration. In some embodiments, the 2-grade or better improvement is assessed by a physician. In some embodiments, the 2-grade or better improvement is assessed by the human patient. In some embodiments, the 2-grade or better improvement is assessed by both a physician and the human patient.

In certain embodiments, a method described herein is better (preferably statistically better) than a comparable method wherein a placebo is administered instead of the botulinum toxin type A at achieving (1) a physician-assessed and/or human patient-assessed platysma prominence scale (e.g., Allergan Platysma Prominence Scale) grade change from moderate or severe prior to the administration of the botulinum toxin type A or placebo to minimal or mild, and (2) a physician-assessed and/or human patient-assessed 2-grade or better improvement on a platysma prominence scale (e.g., the Allergan Platysma Prominence Scale) from moderate or severe prior to the administration of the botulinum toxin type A or placebo to minimal or mild, at 14 days, 30 days, 60 days, 90 days or 120 days post-administration. In some embodiments, the platysma prominence scale (e.g., Allergan Platysma Prominence Scale) grade change is assessed by a physician. In some embodiments, the platysma prominence scale (e.g., Allergan Platysma Prominence Scale) grade change is assessed by the human patient. In some embodiments, the platysma prominence scale (e.g., Allergan Platysma Prominence Scale) grade change is assessed by both a physician and the human patient. In some embodiments, the 2-grade or better improvement is assessed by a physician. In some embodiments, the 2-grade or better improvement is assessed by the human patient. In some embodiments, the 2-grade or better improvement is assessed by both a physician and the human patient. In some embodiments, the grade change and the 2-grade or better improvement are assessed by a physician. In some embodiments, the grade change and the 2-grade or better improvement are assessed by the human patient. In some embodiments, the grade change and the 2-grade or better improvement are assessed by both a physician and the human patient.

In certain embodiments, a method described herein is better (preferably statistically better) than a comparable method wherein a placebo is administered instead of the botulinum toxin type A at achieving a physician-assessed and/or human patient-assessed grade of minimal or mild and at least a 2-grade improvement from baseline on a platysma prominence scale (e.g., the Allergan Platysma Prominence Scale), at 14 days, 30 days, 60 days, 90 days or 120 days post-administration. The baseline is assessed prior to the administration of the botulinum toxin type A. In specific embodiments, the baseline is assessed on the day of but prior to the administration of the botulinum toxin type A. In specific embodiments, the baseline is a grade of moderate or severe on the platysma prominence scale (e.g., Allergan Platysma Prominence Scale). In some embodiments, the achievement of a grade of minimal or mild and the at least 2-grade improvement is assessed by a physician. In some embodiments, the achievement of a grade of minimal or mild and the at least 2-grade improvement is assessed by the human patient. In some embodiments, the achievement of a grade of minimal or mild and the at least 2-grade improvement is assessed by both a physician and the human patient.

In certain embodiments, the comparison of the method described herein relative to the comparable method is assessed at a population level based on the percentage of human patients treated with the method (or the comparable method, as the case may be) who have obtained and maintained the cosmetic effect.

In various aspects and embodiments, a cosmetic effect of a method described herein peaks around or within 30 days following the administration of the botulinum toxin type A, wherein the cosmetic effect comprises a physician-assessed and/or human patient-assessed platysma prominence scale (e.g., Allergan Platysma Prominence Scale) grade change from moderate or severe prior to the administration of the botulinum toxin type A to minimal or mild following the administration of the botulinum toxin type A. In some embodiments, the cosmetic effect of a method described herein peaks around or within 14 days (including at 14 days) following administration of the botulinum toxin type A. In some embodiments wherein both sides of a human patient's neck are treated with a method described herein, the cosmetic effect comprises achieving the foregoing effect on both sides of the human patient's neck. In other embodiments wherein both sides of a human patient's neck are treated with a method described herein, the cosmetic effect comprises achieving the foregoing effect on at least one side of the human patient's neck. In some embodiments, the grade change is assessed by a physician. In other embodiments, the grade change is assessed by the human patient. In some embodiments, the grade change is assessed by both a physician and the human patient.

In various aspects and embodiments, a cosmetic effect of a method described herein peaks around or within 30 days following the administration of the botulinum toxin type A, wherein the cosmetic effect comprises a physician-assessed and/or human patient-assessed 2-grade or better improvement on a platysma prominence scale (e.g., the Allergan Platysma Prominence Scale) from moderate or severe prior to the administration of the botulinum toxin type A to minimal or mild following the administration of the botulinum toxin type A. In some embodiments, the cosmetic effect of a method described herein peaks around or within 14 days (including at 14 days) following administration of the botulinum toxin type A. In some embodiments wherein both sides of a human patient's neck are treated with a method described herein, the cosmetic effect comprises achieving the foregoing effect on both sides of the human patient's neck. In other embodiments wherein both sides of a human patient's neck are treated with a method described herein, the cosmetic effect comprises achieving the foregoing effect on at least one side of the human patient's neck. In some embodiments, the 2-grade or better improvement is assessed by a physician. In other embodiments, the 2-grade or better improvement is assessed by the human patient. In some embodiments, the 2-grade or better improvement is assessed by both a physician and the human patient.

In various aspects and embodiments, a cosmetic effect of a method described herein peaks around or within 30 days following the administration of the botulinum toxin type A, wherein the cosmetic effect comprises a physician-assessed and/or human patient-assessed 1-grade or better improvement on a platysma prominence scale (e.g., the Allergan Platysma Prominence Scale) from moderate or severe prior to the administration of the botulinum toxin type A to mild or moderate following the administration of the botulinum toxin type A. In some embodiments, the cosmetic effect of a method described herein peaks around or within 14 days (including at 14 days) following administration of the botulinum toxin type A. In some embodiments wherein both sides of a human patient's neck are treated with a method described herein, the cosmetic effect comprises achieving the foregoing effect on both sides of the human patient's neck. In other embodiments wherein both sides of a human patient's neck are treated with a method described herein, the cosmetic effect comprises achieving the foregoing effect on at least one side of the human patient's neck. In some embodiments, the 1-grade or better improvement is assessed by a physician. In other embodiments, the 1-grade or better improvement is assessed by the human patient. In some embodiments, the 1-grade or better improvement is assessed by both a physician and the human patient.

In various aspects and embodiments, a cosmetic effect of a method described herein peaks around or within 30 days following the administration of the botulinum toxin type A, wherein the cosmetic effect comprises: (1) a physician-assessed and/or human patient-assessed platysma prominence scale (e.g., Allergan Platysma Prominence Scale) grade change from moderate or severe prior to the administration of the botulinum toxin type A to minimal or mild following the administration of the botulinum toxin type A; and (2) a physician-assessed and/or human patient-assessed 2-grade or better improvement on a platysma prominence scale (e.g., the Allergan Platysma Prominence Scale) from moderate or severe prior to the administration of the botulinum toxin type A to minimal or mild following the administration of the botulinum toxin type A. In some embodiments, the cosmetic effect of a method described herein peaks around or within 14 days (including at 14 days) following administration of the botulinum toxin type A. In some embodiments, the platysma prominence scale (e.g., Allergan Platysma Prominence Scale) grade change is assessed by a physician. In some embodiments, the platysma prominence scale (e.g., Allergan Platysma Prominence Scale) grade change is assessed by the human patient. In some embodiments, the platysma prominence scale (e.g., Allergan Platysma Prominence Scale) grade change is assessed by both a physician and the human patient. In some embodiments, the 2-grade or better improvement is assessed by a physician. In some embodiments, the 2-grade or better improvement is assessed by the human patient. In some embodiments, the 2-grade or better improvement is assessed by both a physician and the human patient. In some embodiments, the platysma prominence scale (e.g., Allergan Platysma Prominence Scale) grade change and the 2-grade or better improvement are assessed by a physician. In some embodiments, the platysma prominence scale (e.g., Allergan Platysma Prominence Scale) grade change and the 2-grade or better improvement are assessed by the human patient. In some embodiments, the platysma prominence scale (e.g., Allergan Platysma Prominence Scale) grade change and the 2-grade or better improvement are assessed by both a physician and the human patient. In some embodiments wherein both sides of a human patient's neck are treated with a method described herein, the cosmetic effect comprises achieving the foregoing effect on both sides of the human patient's neck. In other embodiments wherein both sides of a human patient's neck are treated with a method described herein, the cosmetic effect comprises achieving the foregoing effect on at least one side of the human patient's neck.

In various aspects and embodiments, a cosmetic effect of a method described herein peaks around or within 30 days following the administration of the botulinum toxin type A, wherein the cosmetic effect comprises achieving a physician-assessed and/or human patient-assessed grade of minimal or mild and at least a 2-grade improvement from baseline on a platysma prominence scale (e.g., Allergan Platysma Prominence Scale). The baseline is assessed prior to the administration of the botulinum toxin type A. In specific embodiments, the baseline is assessed on the day of but prior to the administration of the botulinum toxin type A. In specific embodiments, the baseline is a grade of moderate or severe on the platysma prominence scale (e.g., Allergan Platysma Prominence Scale). In some embodiments, the cosmetic effect of a method described herein peaks around or within 14 days (including at 14 days) following administration of the botulinum toxin type A. In some embodiments, the achievement of a grade of minimal or mild and the at least 2-grade improvement is assessed by a physician. In some embodiments, the achievement of a grade of minimal or mild and the at least 2-grade improvement is assessed by the human patient. In some embodiments, the achievement of a grade of minimal or mild and the at least 2-grade improvement is assessed by both a physician and the human patient. In some embodiments wherein both sides of a human patient's neck are treated with a method described herein, the cosmetic effect comprises achieving the foregoing effect on both sides of the human patient's neck. In other embodiments wherein both sides of a human patient's neck are treated with a method described herein, the cosmetic effect comprises achieving the foregoing effect on at least one side of the human patient's neck.

In certain embodiments, the peak time of the cosmetic effect is assessed at a population level based on the percentage of human patients treated with the method who have obtained and maintained the cosmetic effect.

In various aspects and embodiments, the duration of a cosmetic effect of a method described herein is at least 30 days, at least 60 days, at least 90 days, or at least 120 days following the administration of the botulinum toxin type A, or ranges from about 30 days to about 60 days, from about 30 days to about 90 days, from about 30 days to about 120 days, from about 60 days to about 90 days, from about 60 days to about 120 days, or from about 90 days to about 120 days following the administration of the botulinum toxin type A, wherein the cosmetic effect comprises a physician-assessed and/or human patient-assessed platysma prominence scale (e.g., Allergan Platysma Prominence Scale) grade change from moderate or severe prior to the administration of the botulinum toxin type A to minimal or mild following the administration of the botulinum toxin type A. In some embodiments, the grade change is assessed by a physician. In some embodiments, the grade change is assessed by the human patient. In some embodiments, the grade change is assessed by both the physician and the human patient. In some embodiments wherein both sides of a human patient's neck are treated with a method described herein, the cosmetic effect comprises achieving the foregoing effect on both sides of the human patient's neck. In other embodiments wherein both sides of a human patient's neck are treated with a method described herein, the cosmetic effect comprises achieving the foregoing effect on at least one side of the human patient's neck.

In various aspects and embodiments, the duration of a cosmetic effect of a method described herein is at least 30 days, at least 60 days, at least 90 days, or at least 120 days following the administration of the botulinum toxin type A, or ranges from about 30 days to about 60 days, from about 30 days to about 90 days, from about 30 days to about 120 days, from about 60 days to about 90 days, from about 60 days to about 120 days, or from about 90 days to about 120 days following the administration of the botulinum toxin type A, wherein the cosmetic effect comprises a physician-assessed and/or human patient-assessed 2-grade or better improvement on a platysma prominence scale (e.g., the Allergan Platysma Prominence Scale) from moderate or severe prior to the administration of the botulinum toxin type A to minimal or mild following the administration of the botulinum toxin type A. In some embodiments, the 2-grade or better improvement is assessed by a physician. In some embodiments, the 2-grade or better improvement is assessed by the human patient. In some embodiments, the 2-grade or better improvement is assessed by both the physician and the human patient. In some embodiments wherein both sides of a human patient's neck are treated with a method described herein, the cosmetic effect comprises achieving the foregoing effect on both sides of the human patient's neck. In other embodiments wherein both sides of a human patient's neck are treated with a method described herein, the cosmetic effect comprises achieving the foregoing effect on at least one side of the human patient's neck.

In various aspects and embodiments, the duration of a cosmetic effect of a method described herein is at least 30 days, at least 60 days, at least 90 days, or at least 120 days following the administration of the botulinum toxin type A, or ranges from about 30 days to about 60 days, from about 30 days to about 90 days, from about 30 days to about 120 days, from about 60 days to about 90 days, from about 60 days to about 120 days, or from about 90 days to about 120 days following the administration of the botulinum toxin type A, wherein the cosmetic effect comprises a physician-assessed and/or human patient-assessed 1-grade or better improvement on a platysma prominence scale (e.g., the Allergan Platysma Prominence Scale) from moderate or severe prior to the administration of the botulinum toxin type A to mild or moderate following the administration of the botulinum toxin type A. In some embodiments, the 1-grade or better improvement is assessed by a physician. In some embodiments, the 1-grade or better improvement is assessed by the human patient. In some embodiments, the 1-grade or better improvement is assessed by both the physician and the human patient. In some embodiments wherein both sides of a human patient's neck are treated with a method described herein, the cosmetic effect comprises achieving the foregoing effect on both sides of the human patient's neck. In other embodiments wherein both sides of a human patient's neck are treated with a method described herein, the cosmetic effect comprises achieving the foregoing effect on at least one side of the human patient's neck.

In various aspects and embodiments, the duration of a cosmetic effect of a method described herein is at least 30 days, at least 60 days, at least 90 days, or at least 120 days following the administration of the botulinum toxin type A, or ranges from about 30 days to about 60 days, from about 30 days to about 90 days, from about 30 days to about 120 days, from about 60 days to about 90 days, from about 60 days to about 120 days, or from about 90 days to about 120 days following the administration of the botulinum toxin type A, wherein the cosmetic effect comprises: (1) a physician-assessed and/or human patient-assessed platysma prominence scale (e.g., Allergan Platysma Prominence Scale) grade change from moderate or severe prior to the administration of the botulinum toxin type A to minimal or mild following the administration of the botulinum toxin type A; and (2) a physician-assessed and/or human patient-assessed 2-grade or better improvement on a platysma prominence scale (e.g., the Allergan Platysma Prominence Scale) from moderate or severe prior to the administration of the botulinum toxin type A to minimal or mild following the administration of the botulinum toxin type A. In some embodiments, the platysma prominence scale (e.g., Allergan Platysma Prominence Scale) grade change is assessed by a physician. In some embodiments, the platysma prominence scale (e.g., Allergan Platysma Prominence Scale) grade change is assessed by the human patient. In some embodiments, the platysma prominence scale (e.g., Allergan Platysma Prominence Scale) grade change is assessed by both a physician and the human patient. In some embodiments, the 2-grade or better improvement is assessed by a physician. In some embodiments, the 2-grade or better improvement is assessed by the human patient. In some embodiments, the 2-grade or better improvement is assessed by both a physician and the human patient. In some embodiments, the platysma prominence scale (e.g., Allergan Platysma Prominence Scale) grade change and the 2-grade or better improvement are assessed by a physician. In some embodiments, the platysma prominence scale (e.g., Allergan Platysma Prominence Scale) grade change and the 2-grade or better improvement are assessed by the human patient. In some embodiments, the platysma prominence scale (e.g., Allergan Platysma Prominence Scale) grade change and the 2-grade or better improvement are assessed by both a physician and the human patient. In some embodiments wherein both sides of a human patient's neck are treated with a method described herein, the cosmetic effect comprises achieving the foregoing effect on both sides of the human patient's neck. In other embodiments wherein both sides of a human patient's neck are treated with a method described herein, the cosmetic effect comprises achieving the foregoing effect on at least one side of the human patient's neck.

In various aspects and embodiments, the duration of a cosmetic effect of a method described herein is at least 30 days, at least 60 days, at least 90 days, or at least 120 days following the administration of the botulinum toxin type A, or ranges from about 30 days to about 60 days, from about 30 days to about 90 days, from about 30 days to about 120 days, from about 60 days to about 90 days, from about 60 days to about 120 days, or from about 90 days to about 120 days following the administration of the botulinum toxin type A, wherein the cosmetic effect comprises achieving a physician-assessed and/or human patient-assessed grade of minimal or mild and at least a 2-grade improvement from baseline on a platysma prominence scale (e.g., Allergan Platysma Prominence Scale). The baseline is assessed prior to the administration of the botulinum toxin type A. In specific embodiments, the baseline is assessed on the day of but prior to the administration of the botulinum toxin type A. In specific embodiments, the baseline is a grade of moderate or severe on the platysma prominence scale (e.g., Allergan Platysma Prominence Scale). In some embodiments, the achievement of a grade of minimal or mild and the at least 2-grade improvement is assessed by a physician. In some embodiments, the achievement of a grade of minimal or mild and the at least 2-grade improvement is assessed by the human patient. In some embodiments, the achievement of a grade of minimal or mild and the at least 2-grade improvement is assessed by both a physician and the human patient. In some embodiments wherein both sides of a human patient's neck are treated with a method described herein, the cosmetic effect comprises achieving the foregoing effect on both sides of the human patient's neck. In other embodiments wherein both sides of a human patient's neck are treated with a method described herein, the cosmetic effect comprises achieving the foregoing effect on at least one side of the human patient's neck.

In certain embodiments, the duration of the cosmetic effect is assessed at a population level based on whether the percentage of human patients treated with the method who have obtained and maintained the cosmetic effect is above a set threshold (e.g., 5%, 10%, 15%, or 20%).

In various aspects and embodiments, a method described herein is better (preferably statistically better) than a comparable method wherein a placebo is administered instead of a botulinum toxin type A at achieving a physician-assessed and/or human patient-assessed grade of minimal or mild on a platysma prominence scale (e.g., the Allergan Platysma Prominence Scale) at 14 days, 30 days, 60 days, 90 days or 120 days post-administration. In some embodiments, the grade on the platysma prominence scale is assessed by a physician. In some embodiments, the grade on the platysma prominence scale is assessed by the human patient. In some embodiments, the grade on the platysma prominence scale is assessed by both a physician and the human patient. In some embodiments wherein both sides of a human patient's neck are treated with a method described herein, the cosmetic effect comprises achieving the foregoing effect on both sides of the human patient's neck. In other embodiments wherein both sides of a human patient's neck are treated with a method described herein, the cosmetic effect comprises achieving the foregoing effect on at least one side of the human patient's neck.

In various aspects and embodiments, a method described herein is better (preferably statistically better) than a comparable method wherein a placebo is administered instead of a botulinum toxin type A at achieving a physician-assessed and/or human patient-assessed platysma prominence scale (e.g., Allergan Platysma Prominence Scale) grade change from moderate or severe prior to the administration of the botulinum toxin type A or placebo to minimal or mild at 14 days, 30 days, 60 days, 90 days or 120 days post-administration. In some embodiments, the grade change is assessed by a physician. In some embodiments, the grade change is assessed by the human patient. In some embodiments, the grade change is assessed by both a physician and the human patient. In some embodiments wherein both sides of a human patient's neck are treated with a method described herein, the cosmetic effect comprises achieving the foregoing effect on both sides of the human patient's neck. In other embodiments wherein both sides of a human patient's neck are treated with a method described herein, the cosmetic effect comprises achieving the foregoing effect on at least one side of the human patient's neck.

In various aspects and embodiments, a method described herein is better (preferably statistically better) than a comparable method wherein a placebo is administered instead of a botulinum toxin type A at achieving a physician-assessed and/or human patient-assessed 2-grade or better improvement on a platysma prominence scale (e.g., the Allergan Platysma Prominence Scale) from moderate or severe prior to the administration of the botulinum toxin type A or placebo to minimal or mild at 14 days, 30 days, 60 days, 90 days or 120 days post-administration. In some embodiments, the 2-grade or better improvement is assessed by a physician. In some embodiments, the 2-grade or better improvement is assessed by the human patient. In some embodiments, the 2-grade or better improvement is assessed by both a physician and the human patient. In some embodiments wherein both sides of a human patient's neck are treated with a method described herein, the cosmetic effect comprises achieving the foregoing effect on both sides of the human patient's neck. In other embodiments wherein both sides of a human patient's neck are treated with a method described herein, the cosmetic effect comprises achieving the foregoing effect on at least one side of the human patient's neck.

In various aspects and embodiments, a method described herein is better (preferably statistically better) than a comparable method wherein a placebo is administered instead of a botulinum toxin type A at achieving a physician-assessed and/or human patient-assessed 1-grade or better improvement on a platysma prominence scale (e.g., the Allergan Platysma Prominence Scale) from moderate or severe prior to the administration of the botulinum toxin type A or placebo to mild or moderate at 14 days, 30 days, 60 days, 90 days or 120 days post-administration. In some embodiments, the 1-grade or better improvement is assessed by a physician. In some embodiments, the 1-grade or better improvement is assessed by the human patient. In some embodiments, the 1-grade or better improvement is assessed by both a physician and the human patient. In some embodiments wherein both sides of a human patient's neck are treated with a method described herein, the cosmetic effect comprises achieving the foregoing effect on both sides of the human patient's neck. In other embodiments wherein both sides of a human patient's neck are treated with a method described herein, the cosmetic effect comprises achieving the foregoing effect on at least one side of the human patient's neck.

In various aspects and embodiments, a method described herein is better (preferably statistically better) than a comparable method wherein a placebo is administered instead of a botulinum toxin type A at achieving (1) a physician-assessed and/or human patient-assessed platysma prominence scale (e.g., Allergan Platysma Prominence Scale) grade change from moderate or severe prior to the administration of the botulinum toxin type A or placebo to minimal or mild, and (2) a physician-assessed and/or human patient-assessed 2-grade or better improvement on a platysma prominence scale (e.g., the Allergan Platysma Prominence Scale) from moderate or severe prior to the administration of the botulinum toxin type A or placebo to minimal or mild, at 14 days, 30 days, 60 days, 90 days or 120 days post-administration. In some embodiments, the platysma prominence scale (e.g., Allergan Platysma Prominence Scale) grade change is assessed by a physician. In other embodiments, the platysma prominence scale (e.g., Allergan Platysma Prominence Scale) grade change is assessed by the human patient. In some embodiments, the platysma prominence scale (e.g., Allergan Platysma Prominence Scale) grade change is assessed by both a physician and the human patient. In some embodiments, the 2-grade or better improvement is assessed by a physician. In some embodiments, the 2-grade or better improvement is assessed by the human patient. In some embodiments, the 2-grade or better improvement is assessed by both a physician and the human patient. In some embodiments, the platysma prominence scale (e.g., Allergan Platysma Prominence Scale) grade change and the 2-grade or better improvement are assessed by a physician. In some embodiments, the platysma prominence scale (e.g., Allergan Platysma Prominence Scale) grade change and the 2-grade or better improvement are assessed by the human patient. In some embodiments, the platysma prominence scale (e.g., Allergan Platysma Prominence Scale) grade change and the 2-grade or better improvement are assessed by both a physician and the human patient. In some embodiments wherein both sides of a human patient's neck are treated with a method described herein, the cosmetic effect comprises achieving the foregoing effect on both sides of the human patient's neck. In other embodiments wherein both sides of a human patient's neck are treated with a method described herein, the cosmetic effect comprises achieving the foregoing effect on at least one side of the human patient's neck.

In various aspects and embodiments, a method described herein is better (preferably statistically better) than a comparable method wherein a placebo is administered instead of a botulinum toxin type A at achieving a physician-assessed and/or human patient-assessed grade of minimal or mild and at least a 2-grade improvement from baseline on a platysma prominence scale (e.g., the Allergan Platysma Prominence Scale), at 14 days, 30 days, 60 days, 90 days or 120 days post-administration. The baseline is assessed prior to the administration of the botulinum toxin type A. In specific embodiments, the baseline is assessed on the day of but prior to the administration of the botulinum toxin type A. In specific embodiments, the baseline is a grade of moderate or severe on the platysma prominence scale (e.g., Allergan Platysma Prominence Scale). In some embodiments, the achievement of a grade of minimal or mild and the at least 2-grade improvement is assessed by a physician. In some embodiments, the achievement of a grade of minimal or mild and the at least 2-grade improvement is assessed by the human patient. In some embodiments, the achievement of a grade of minimal or mild and the at least 2-grade improvement is assessed by both a physician and the human patient. In some embodiments wherein both sides of a human patient's neck are treated with a method described herein, the cosmetic effect comprises achieving the foregoing effect on both sides of the human patient's neck. In other embodiments wherein both sides of a human patient's neck are treated with a method described herein, the cosmetic effect comprises achieving the foregoing effect on at least one side of the human patient's neck.

In preferred embodiments, the comparison of the method described herein relative to the comparable method is assessed at 14 days post-administration. In certain embodiments, the comparison of the method described herein relative to the comparable method is assessed at 30 days post-administration. In certain embodiments, the comparison of the method described herein relative to the comparable method is assessed at 60 days post-administration.

In certain embodiments, the comparison of the method described herein relative to the comparable method is assessed at a population level based on the percentage of human patients treated with the method (or the comparable method, as the case may be) who have obtained and maintained the cosmetic effect. In certain embodiments, the method described herein is at least 25% better than the comparable method, for examples, at least 50% better, at least 75% better, or at least 90% better than the comparable method. In certain embodiments, the method described herein is at least 100% better than the comparable method. In certain embodiments, the method described herein is at least 2-fold better than the comparable method, for examples, at least 3-fold better, at least 5-fold better, at least 10-fold better, at least 50-fold better, or at least 90-fold better than the comparable method.

In various aspects and embodiments, a method described herein is better (preferably statistically better) than a comparable method wherein a placebo is administered instead of a botulinum toxin type A when assessed by the Appearance of Neck and Lower Face Questionnaire (ANLFQ)-Satisfaction, Follow-up, Item 5, which evaluates satisfaction of the patient with the effect of the treatment at 7 days, 14 days, 30 days, 60 days, 90 days or 120 days post-administration. In various aspects and embodiments, a method described herein is better (preferably statistically better) than a comparable method wherein a placebo is administered instead of a botulinum toxin type A when assessed by the Bother Assessment Scale-Platysma Prominence (BAS-PP) Item 1 and Item 2, which assesses how bothered the patient is by the appearance of their platysma bands and their jawline, respectively, at 7 days, 14 days, 30 days, 60 days, 90 days or 120 days post administration. In certain embodiments, the comparison of the method described herein relative to the comparable method is assessed at a population level based on the percentage of human patients treated with the method (or the comparable method, as the case may be) who have answered “satisfied” or “very satisfied” on the ANLFQ-Satisfaction, Follow-up, Item 5; or “not at all bothered” or “a little bothered” on the BAS-PP, Item 1 and Item 2, questionnaire (as the case may be). In certain embodiments, the method described herein is at least 25% better than the comparable method, for examples, at least 50% better, at least 75% better, or at least 90% better than the comparable method. In certain embodiments, the method described herein is at least 100% better than the comparable method. In certain embodiments, the method described herein is at least 2-fold better than the comparable method, for examples, at least 3-fold better, at least 5-fold better, at least 10-fold better, at least 50-fold better, or at least 90-fold better than the comparable method.

“Treatment-emergent adverse event” or “TEAE” is a term of art as understood by those of ordinary skill in the art. In specific embodiments, a TEAE is an adverse event that begins after the first dose of the composition comprising botulinum toxin type A or an adverse event that is present before the first dose of the composition comprising botulinum toxin type A but increases in severity or becomes serious after the first dose of the composition comprising botulinum toxin type A. In specific embodiments, a TEAE is an adverse event wherein the onset is after the first dose of the composition comprising botulinum toxin type A.

In various aspects and embodiments, a method described herein is associated with less than 25% treatment-emergent adverse events (TEAEs), less than 30% TEAEs, less than 35% TEAEs, or less than 40% TEAEs (e.g., when assessed at a population level). In specific embodiments, the above-mentioned safety result is obtained after one treatment cycle. In specific embodiments, the above-mentioned safety result is obtained after two treatment cycles. In specific embodiments, the above-mentioned safety result is obtained after three treatment cycles. In specific embodiments, the above-mentioned safety result is obtained after four treatment cycles. See descriptions below regarding treatment cycles.

In various aspects and embodiments, a method described herein results in an incidence rate of less than 2% of TEAEs, less than 3%, less than 4%, less than 5%, less than 10%, or less than 16% of TEAEs related to the composition comprising botulinum toxin type A (e.g., when assessed at a population level). In various aspects and embodiments, a method described herein results in an incidence rate of less than 5% or less than 2% of TEAEs related to the composition comprising botulinum toxin type A (e.g., when assessed at a population level). In specific embodiments, the above-mentioned safety result is obtained after one treatment cycle. In specific embodiments, the above-mentioned safety result is obtained after two treatment cycles. In specific embodiments, the above-mentioned safety result is obtained after three treatment cycles. In specific embodiments, the above-mentioned safety result is obtained after four treatment cycles. See descriptions below regarding treatment cycles.

The determination of whether an adverse event is severe or not can be based on a standard set forth by a person of ordinary skill in the art. In specific embodiments, a severe adverse event is a type of adverse event that interrupts usual activities of daily living, or significantly affects clinical status, or may require intensive therapeutic intervention. In specific embodiments, a severe adverse event is a type of adverse event that causes considerable interference with the subject's usual activities and may be incapacitating or life threatening.

In various aspects and embodiments, a method described herein results in an incidence rate of less than 1% or less than 0.5% of treatment-related severe TEAEs, or no incidence of treatment-related severe TEAE. In various aspects and embodiments, a method described herein results in an incidence rate of less than 1%, less than 0.9%, less than 0.8%, less than 0.7%, or less than 0.6% of treatment-related severe TEAEs.

In various aspects and embodiments, a method described herein results in an incidence rate of less than 0.5% of treatment-related severe TEAEs, for examples, less than 0.4%, less than 0.3%, less than 0.2%, or less than 0.1% of treatment-related severe TEAEs. In various aspects and embodiments, a method described herein results in no incidence of treatment-related severe TEAE. In specific embodiments, the above-mentioned safety result is obtained after one treatment cycle. In specific embodiments, the above-mentioned safety result is obtained after two treatment cycles. In specific embodiments, the above-mentioned safety result is obtained after three treatment cycles. In specific embodiments, the above-mentioned safety result is obtained after four treatment cycles. See descriptions below regarding treatment cycles.

In various aspects and embodiments, a method described herein results in an incidence rate of less than 5%, less than 4%, less than 3% less than 2%, less than 1%, less than 0.5%, less than 0.3%, or less than 0.1% of adverse events of special interest (AESIs), or no incidence of AESI. In specific embodiments, AESIs include aspiration, aspiration pneumonia, dry mouth, dysphagia, dyspnea, facial paresis, and muscular weakness (i.e., neck muscle weakness). In a specific embodiment, the AESI(s) is aspiration. In another specific embodiment, the AESI(s) is aspiration pneumonia. In another specific embodiment, the AESI(s) is dry mouth. In another specific embodiment, the AESI(s) is dysphagia. In another specific embodiment, the AESI(s) is dyspnea. In another specific embodiment, the AESI(s) is facial paresis. In another specific embodiment, the AESI(s) is muscular weakness (i.e., neck muscle weakness). In specific embodiments, the above-mentioned safety result is obtained after one treatment cycle. In specific embodiments, the above-mentioned safety result is obtained after two treatment cycles. In specific embodiments, the above-mentioned safety result is obtained after three treatment cycles. In specific embodiments, the above-mentioned safety result is obtained after four treatment cycles. See descriptions below regarding treatment cycles.

In various aspects and embodiments, a method described herein results in an incidence rate of less than 10%, less than 5%, less than 2%, or less than 1.5% of injection site bruising. In specific embodiments, the above-mentioned safety result is obtained after one treatment cycle. In specific embodiments, the above-mentioned safety result is obtained after two treatment cycles. In specific embodiments, the above-mentioned safety result is obtained after three treatment cycles. In specific embodiments, the above-mentioned safety result is obtained after four treatment cycles. See descriptions below regarding treatment cycles.

In various aspects and embodiments, a method described herein results in an incidence rate of less than 1%, less than 0.5% or less than 0.1% of neck muscle weakness, or no incidence of neck muscle weakness. In specific embodiments, the above-mentioned safety result is obtained after one treatment cycle. In specific embodiments, the above-mentioned safety result is obtained after two treatment cycles. In specific embodiments, the above-mentioned safety result is obtained after three treatment cycles. In specific embodiments, the above-mentioned safety result is obtained after four treatment cycles. See descriptions below regarding treatment cycles.

In various aspects and embodiments, a method described herein results in an incidence rate of less than 2%, less than 1%, less than 0.5%, or less than 0.1% of dysphagia, or no incidence of dysphagia. In specific embodiments, the above-mentioned safety result is obtained after one treatment cycle. In specific embodiments, the above-mentioned safety result is obtained after two treatment cycles. In specific embodiments, the above-mentioned safety result is obtained after three treatment cycles. In specific embodiments, the above-mentioned safety result is obtained after four treatment cycles. See descriptions below regarding treatment cycles.

In various aspects and embodiments, a method described herein results in an incidence rate of less than 5%, less than 2%, less than 1%, or less than 0.5% of injection site hematoma, or no incidence of injection site hematoma. In specific embodiments, the above-mentioned safety result is obtained after one treatment cycle. In specific embodiments, the above-mentioned safety result is obtained after two treatment cycles. In specific embodiments, the above-mentioned safety result is obtained after three treatment cycles. In specific embodiments, the above-mentioned safety result is obtained after four treatment cycles. See descriptions below regarding treatment cycles.

In various aspects and embodiments, a method described herein results in a reduced incidence of adverse effects as compared to a comparable method for treating platysma prominence (e.g., for improving the appearance of platysma prominence such as the appearance of platysma bands) that comprises injecting one or more neck band doses of the botulinum toxin type A to one or more neck bands of a human subject's neck but does not comprise injecting a jawline dose of the botulinum toxin type A to the human subject's neck.

In various aspects and embodiments, a method described herein results in a reduced incidence of adverse effects as compared to a comparable method for treating platysma prominence (e.g., for improving the appearance of platysma prominence such as the appearance of platysma bands) that comprises injecting one or more neck band doses of the botulinum toxin type A to one or more neck bands of a human subject's neck and injecting one or more jawline doses of the botulinum toxin type A to one or both sides of the jawline of the human subject's neck, wherein at least one neck band dose is higher than at least one jawline dose.

In various aspects and embodiments, a method described herein results in a reduced incidence of adverse effects as compared to a comparable method for treating platysma prominence (e.g., for improving the appearance of platysma prominence such as the appearance of platysma bands) that comprises injecting a neck band dose of the botulinum toxin type A that is higher than the neck band dose injected in accordance with the method described herein (for example, injecting a neck band dose that is higher than 5 units of the botulinum toxin type A per neck band).

In various aspects and embodiments, a method described herein results in a reduced incidence of adverse effects as compared to a comparable method for treating platysma prominence (e.g., for improving the appearance of platysma prominence such as the appearance of platysma bands) that comprises injecting a neck band dose of the botulinum toxin type A that has a higher per injection site dose than the neck band dose injected in accordance with the method described herein (for example, injecting a neck band dose that has a per injection site dose of higher than 1 unit of the botulinum toxin type A).

In various aspects and embodiments, a method described herein results in a reduced incidence of adverse effects as compared to a comparable method for treating platysma prominence (e.g., for improving the appearance of platysma prominence such as the appearance of platysma bands) that comprises injecting a jawline dose of the botulinum toxin type A that is higher than the jawline dose injected in accordance with the method described herein (for example, injecting a jawline dose that is higher than 8 units of the botulinum toxin type A per side of the jawline).

In various aspects and embodiments, a method described herein results in a reduced incidence of adverse effects as compared to a comparable method for treating platysma prominence (e.g., for improving the appearance of platysma prominence such as the appearance of platysma bands) that comprises injecting a jawline dose of the botulinum toxin type A that has a higher per injection site dose than the jawline dose injected in accordance with the method described herein (for example, injecting a jawline dose that has a per injection site dose of higher than 2 units of the botulinum toxin type A).

In various aspects and embodiments, a total dose of 18 units or less than 18 units of the botulinum toxin type A is injected as a jawline dose and one or two neck band doses to one side of the human patient's neck in accordance of a method described herein, and the method results in a reduced incidence of adverse effects as compared to a comparable method for treating platysma prominence (e.g., for improving the appearance of platysma prominence such as the appearance of platysma bands) wherein more than 18 units of the botulinum toxin type A is injected to one side of a human subject's neck (for example, as a jawline dose and one or more, e.g., one or two, neck band doses).

In various aspects and embodiments, a total dose of 18 units of the botulinum toxin type A is injected as a jawline dose and two neck band doses to one side of the human patient's neck in accordance of a method described herein, and the method results in a reduced incidence of adverse effects as compared to a comparable method for treating platysma prominence (e.g., for improving the appearance of platysma prominence such as the appearance of platysma bands) wherein more than 18 units of the botulinum toxin type A is injected to one side of a human subject's neck (for example, as a jawline dose and one or more, e.g., one or two, neck band doses).

In various aspects and embodiments, a total dose of 13 units of the botulinum toxin type A is injected as a jawline dose and a neck band dose to one side of the human patient's neck in accordance of a method described herein, and the method results in a reduced incidence of adverse effects as compared to a comparable method for treating platysma prominence (e.g., for improving the appearance of platysma prominence such as the appearance of platysma bands) wherein more than 13 units of the botulinum toxin type A is injected to one side of a human subject's neck (for example, as a jawline dose and one or more, e.g., one, neck band dose).

In various aspects and embodiments, a method described herein results in a reduced incidence of adverse effects as compared to a comparable method for treating platysma prominence (e.g., for improving the appearance of platysma prominence such as the appearance of platysma bands) that comprises injecting one or more neck band doses of the botulinum toxin type A to one or more neck bands of a human subject's neck, wherein the one or more neck bands injected with the one or more neck band doses are not limited to continuous vertical neck bands that extend continuously from the human subject's jawline to the lower neck region.

In various aspects and embodiments, a method described herein results in a reduced incidence of adverse effects as compared to a comparable method for treating platysma prominence (e.g., for improving the appearance of platysma prominence such as the appearance of platysma bands) wherein neck band dose(s) of the botulinum toxin type A are injected to a human subject's neck but whether a neck band dose is injected is not dependent on whether the neck band is a continuous vertical neck band.

In various aspects and embodiments, a method described herein results in a reduced incidence of adverse effects as compared to a comparable method for treating platysma prominence (e.g., for improving the appearance of platysma prominence such as the appearance of platysma bands) wherein neck band dose(s) of the botulinum toxin type A are injected to a human subject's neck but are not fixed dose(s) that are only injected along continuous vertical neck band(s).

In various aspects and embodiments, a method described herein results in a reduced incidence of adverse effects as compared to a comparable method for treating platysma prominence (e.g., for improving the appearance of platysma prominence such as the appearance of platysma bands) that comprises injecting neck band doses of the botulinum toxin type A along more than two continuous neck bands on one side of a human subject's neck.

In various aspects and embodiments, a method described herein results in a reduced incidence of adverse effects as compared to a comparable method for treating platysma prominence (e.g., for improving the appearance of platysma prominence such as the appearance of platysma bands) that comprises injecting three or more neck band doses of the botulinum toxin type A to one side of a human subject's neck, with each neck band dose being injected along a different neck band. In certain embodiments, the three or more neck band doses are injected along three or more continuous vertical neck bands. In certain embodiments, the three or more neck band doses are injected along two continuous vertical neck bands and one or more non-continuous neck bands. In certain embodiments, the three or more neck band doses are injected along one continuous vertical neck band and two or more non-continuous vertical neck bands. In certain embodiments, the three or more neck band doses are injected along three or more non-continuous neck bands.

In various aspects and embodiments, a method described herein results in a reduced incidence of adverse effects as compared to a comparable method for treating platysma prominence (e.g., for improving the appearance of platysma prominence such as the appearance of platysma bands) that does not classify platysma prominence according to a platysma prominence scale.

In various aspects and embodiments, a method described herein results in a reduced incidence of adverse effects as compared to a comparable method for treating platysma prominence (e.g., for improving the appearance of platysma prominence such as the appearance of platysma bands) that does not classify platysma prominence according to the Allergan Platysma Prominence Scale (APPS) shown in Table 1 (illustrated in FIG. 1).

In specific embodiments, a method described herein results in a reduced incidence of adverse effects as compared to a comparable method for treating platysma prominence (e.g., for improving the appearance of platysma prominence such as the appearance of platysma bands) that has one, two, three, four, five or more of the features described above that distinguish it from the method described herein.

In specific embodiments, a method described herein results in a reduced incidence of adverse effects as compared to an injection technique or paradigm that is known in the art for treating platysma prominence.

In certain embodiments, the adverse effect is a treatment-emergent adverse event or TEAE (e.g., treatment-related severe TEAE).

In certain embodiments, the adverse effect is one described herein in this disclosure.

In certain embodiments, the adverse effect is selected from the group consisting of aspiration, aspiration pneumonia, dry mouth, dysphagia, dyspnea, facial paresis, muscular weakness, injection site bruising, and injection site hematoma.

In various aspects and embodiments, a method described herein achieves one or more of the above-mentioned efficacy effects. In various aspects and embodiments, a method described herein achieves one or more of the above-mentioned safety effects. In various aspects and embodiments, a method described herein achieves one or more of the above-mentioned efficacy and safety effects. In various aspects and embodiments, a method described herein achieves one or more of the above-mentioned efficacy effects and one or more of the above-mentioned safety effects.

In specific embodiments, a human patient in the context of describing the efficacy and/or safety effect of a method described herein is a subject of a clinical trial, especially when such efficacy and/or safety effect are assessed at a population level.

In specific embodiments, a physician (e.g., a treating physician) in the context of describing the efficacy and/or safety effect of a method described herein is an investigator of a clinical trial, especially when such efficacy and/or safety effect are assessed at a population level.

In various aspects and embodiments, a composition described herein comprising a botulinum toxin type A is a pharmaceutical composition (see Sections 5.1 and 5.2 for more descriptions).

In various aspects and embodiments, the concentration of the botulinum toxin type A in the composition is about 10 to about 100 units per mL. In specific embodiments, the concentration of the botulinum toxin type A in the composition is about 20 to about 60 units per mL. In a specific embodiment, the concentration of the botulinum toxin type A in the composition is about 40 units per mL.

In various aspects and embodiments, the volume of the botulinum toxin type A composition administered to each jawline injection site is about 0.02 mL to about 0.10 mL. In specific embodiments, the volume of the botulinum toxin type A composition administered to each jawline injection site is about 0.04 mL to about 0.08 mL. In specific embodiments, the volume of the botulinum toxin type A composition administered to each jawline injection site is about 0.04 mL to about 0.06 mL. In a specific embodiment, the volume of the botulinum toxin type A composition administered to each jawline injection site is about 0.05 mL.

In various volume of the botulinum toxin type A composition administered to each neck band injection site is about 0.01 mL to about 0.05 mL. In specific embodiments, the volume of the botulinum toxin type A composition administered to each to each neck band is about 0.02 mL to about 0.04 mL. In specific embodiments, the volume of the botulinum toxin type A composition administered to each to each neck band is about 0.02 mL to about 0.03 mL. In a specific embodiment, the volume of the botulinum toxin type A composition administered to each neck band injection site is about 0.025 mL.

In various aspects and embodiments, the botulinum toxin type A used in a method described herein is a botulinum toxin type A described in Section 5.1 or Section 5.2. In specific embodiments, the botulinum toxin type A used in a method described herein is a 900 kD botulinum neurotoxin serotype A (BoNT/A) complex. In a preferred embodiment, the botulinum toxin type A used in a method described herein is onabotulinumtoxin A.

In specific embodiments, the composition comprises a powdered BoNT/A pharmaceutical composition that has been reconstituted with normal saline, wherein prior to the reconstitution, the powered BoNT/A pharmaceutical composition consists of the 900 kDa BoNT/A complex (e.g., about 100 units or 200 units of the 900 kDa BoNT/A complex), human albumin (e.g., about 0.5 mg human albumin per 100 units of the 900 kDa BoNT/A complex), and sodium chloride (e.g., about 0.9 mg sodium chloride per 100 units of the 900 kDa BoNT/A complex).

In specific embodiments, the composition is a liquid. In a specific embodiment, the composition is a liquid pharmaceutical composition that has been reconstituted from a powdered BoNT/A pharmaceutical composition with normal saline, wherein prior to the reconstitution, the powered BoNT/A pharmaceutical composition consists of the 900 kDa BoNT/A complex (e.g., about 100 units or 200 units of the 900 kDa BoNT/A complex), human albumin (e.g., about 0.5 mg human albumin per 100 units of the 900 kDa BoNT/A complex), and sodium chloride (e.g., about 0.9 mg sodium chloride per 100 units of the 900 kDa BoNT/A complex).

In various aspects and embodiments, the injections are not made deeper than about 0.5 inches into a muscle.

In various aspects and embodiments, the injections are administered superficially and intramuscularly with the needle perpendicular to the surface of the skin.

In various aspects and embodiments, no additional dose of the botulinum toxin type A is injected to the human patient's neck (e.g., in the same treatment cycle). In various aspects and embodiments, no additional jawline dose of the botulinum toxin type A is injected to the human patient's neck (e.g., in the same treatment cycle). In various aspects and embodiments, no additional neck band dose of the botulinum toxin type A is injected to the human patient's neck (e.g., in the same treatment cycle). In various aspects and embodiments, no additional jawline dose and no additional neck band dose of the botulinum toxin type A are injected to the human patient's neck (e.g., in the same treatment cycle).

In various aspects and embodiments, the composition is administered to the human patient using a sterile 30-gauge syringe with a 0.5-inch needle. In various aspects and embodiments, the composition is administered to the human patient using a sterile 30-gauge syringe with a 1-inch needle

In specific embodiments, the composition is administered in accordance with the following:

1. Dilute each 100 unit vial of onabotulinumtoxinA (BOTOX®, BOTOX® Cosmetic, or VISTABEL®) with 2.5 mL of sterile, preservative-free saline to achieve 4 units/0.1 mL for the BOTOX®.

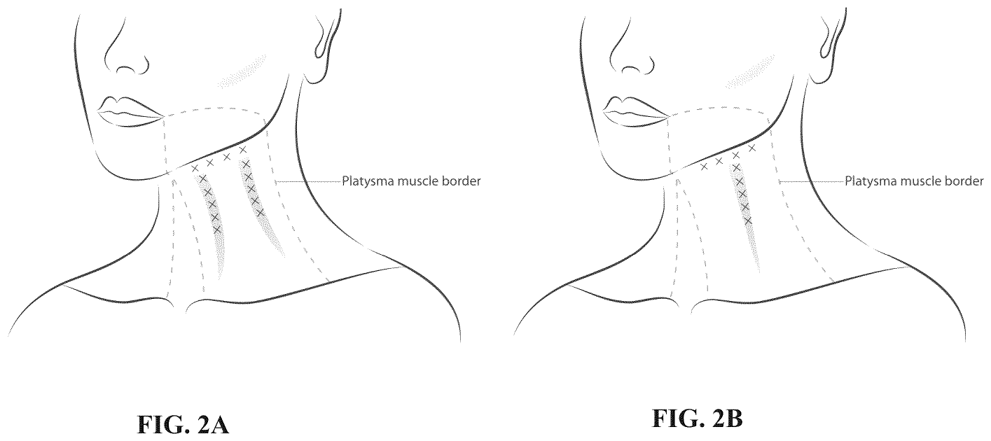

2. Using an appropriately sized sterile syringe, needle, and aseptic technique, inject 2 Units/0.05 mL of reconstituted BOTOX® into 4 sites in the upper segment of platysma muscle, below the jawline on each side. In addition, inject 1 Unit/0.025 mL of reconstituted BOTOX® into 5 sites along each vertical neck band, 1 to 2 vertical neck bands per side (FIGS. 2A and 2B). Depending on platysma prominence severity, the total dose may be 26 Units (1 band/side), 31 Units (1 band one side, 2 bands other side), or 36 Units (2 bands/side).

3. For each side, the 4 jawline injections to the upper platysma muscle should be approximately 1 to 2 cm inferior and parallel to the lower mandibular border. The anterior injection site should be in line with the oral commissure, and the posterior injection site should be slightly anterior to the angle of the mandible. The remaining two injections should be equidistant (approximately 1 to 2 cm apart) between the anterior and posterior injection points. For each vertical neck band identified, 1 to 2 per side, distribute 5 injections vertically approximately 1 to 2 cm apart. The most superior injection site should be approximately 1 to 2 cm inferior to the jawline injections.

4. The platysma muscle is a thin muscle sheet just below the surface of the skin. Therefore, all platysma muscle injections should be administered superficially and intramuscularly with the needle perpendicular to the surface of the skin. For vertical neck band injections, each band should be identified while the patient is contracting their platysma. Gently pinch the band to isolate the muscle from nearby anatomical structures during administration.

5. To reduce injection-related complications, injection should be at least 1 cm inferior to the lower mandibular border. Do not inject into structures deep to the platysma muscle, particularly in the anterior region of the neck.

In various aspects and embodiments, a method described above is repeated for multiple times. In certain embodiments, a method described above is repeated for once. In certain embodiments, a method described above is repeated for twice. In certain embodiments, a method described above is repeated for three times. In specific embodiments, a method described above is repeated no less than every 3 months. In specific embodiments, a method described above is repeated no less than every 12 weeks. In specific embodiments, a method described above is repeated no less than every 84 days. In specific embodiments, a method described above is repeated about every 3 months. In specific embodiments, a method described above is repeated about every 12 weeks. In specific embodiments, a method described above is repeated about every 84 days.

In various aspects and embodiments, the steps of one or more methods described above make up a treatment cycle, and provided herein is an extended treatment method that comprises more than one treatment cycle from the one or more methods described above. In certain embodiments, the extended treatment method comprises 2 treatment cycles. In certain embodiments, the extended treatment method comprises 3 treatment cycles. In certain embodiments, the extended treatment method comprises 4 treatment cycles. In specific embodiments, a subsequent treatment cycle is provided no less than 3 months from the previous treatment cycle. In specific embodiments, a subsequent treatment cycle is provided no less than 12 weeks from the previous treatment cycle. In specific embodiments, a subsequent treatment cycle is provided no less than 84 days from the previous treatment cycle. In specific embodiments, a subsequent treatment cycle is provided about 3 months after the previous treatment cycle. In specific embodiments, a subsequent treatment cycle is provided about 12 weeks after the previous treatment cycle. In specific embodiments, a subsequent treatment cycle is provided about 84 days after the previous treatment cycle.

Also provided herein is a composition comprising a botulinum toxin type A for use in a method described herein for improving (e.g., temporarily improving) the appearance of platysma prominence (e.g., moderate to severe platysma prominence) associated with platysma muscle activity to a human patient.

Further provided herein is use of a composition comprising a botulinum toxin type A for the manufacturing of a medicament for improving (e.g., temporarily improving) the appearance of platysma prominence (e.g., moderate to severe platysma prominence) associated with platysma muscle activity to a human patient in accordance with a method described herein. In specific embodiments, improving (e.g., temporarily improving) the appearance of platysma prominence (e.g., moderate to severe platysma prominence) associated with platysma muscle activity is improving (e.g., temporarily improving) the appearance of platysma bands (e.g., moderate to severe platysma bands) associated with platysma muscle activity. In specific embodiments, improving (e.g., temporarily improving) the appearance of platysma prominence (e.g., moderate to severe platysma prominence) associated with platysma muscle activity is improving (e.g., temporarily improving) the appearance of platysma prominence seen at maximum contraction (e.g., moderate to severe platysma prominence seen at maximum contraction). In specific embodiments, improving (e.g., temporarily improving) the appearance of platysma prominence (e.g., moderate to severe platysma prominence) associated with platysma muscle activity is improving (e.g., temporarily improving) the appearance of platysma bands prominence seen at maximum contraction (e.g., moderate to severe platysma bands prominence seen at maximum contraction). In specific embodiments, improving (e.g., temporarily improving) the appearance of platysma prominence (e.g., moderate to severe platysma prominence) associated with platysma muscle activity is improving (e.g., temporarily improving) the appearance of vertical bands connecting the jaw and neck seen at maximum contraction (e.g., moderate to severe vertical bands connecting the jaw and neck seen at maximum contraction).

Further provided herein is a composition for the improvement of the appearance of platysma prominence substantially as described herein. In specific embodiments, improvement of (e.g., temporary improvement of) the appearance of platysma prominence (e.g., moderate to severe platysma prominence) associated with platysma muscle activity is improvement of (e.g., temporary improvement of) the appearance of platysma bands (e.g., moderate to severe platysma bands) associated with platysma muscle activity. In specific embodiments, improvement of (e.g., temporary improvement of) the appearance of platysma prominence (e.g., moderate to severe platysma prominence) associated with platysma muscle activity is improvement of (e.g., temporary improvement of) the appearance of platysma prominence seen at maximum contraction (e.g., moderate to severe platysma prominence seen at maximum contraction). In specific embodiments, improvement of (e.g., temporary improvement of) the appearance of platysma prominence (e.g., moderate to severe platysma prominence) associated with platysma muscle activity is improvement of (e.g., temporary improvement of) the appearance of platysma bands prominence seen at maximum contraction (e.g., moderate to severe platysma bands prominence seen at maximum contraction). In specific embodiments, improvement of (e.g., temporary improvement of) the appearance of platysma prominence (e.g., moderate to severe platysma prominence) associated with platysma muscle activity is improvement of (e.g., temporary improvement of) the appearance of vertical bands connecting the jaw and neck seen at maximum contraction (e.g., moderate to severe vertical bands connecting the jaw and neck seen at maximum contraction).

Further provided is use of a composition comprising a botulinum toxin type A substantially as described herein.

### 6. ILLUSTRATIVE EMBODIMENTS

The present disclosure includes the following non-limiting illustrative embodiments:

Embodiment 1: A method for improving the appearance of platysma prominence associated with platysma muscle activity to a human patient, comprising:
(A) quantifying the number of continuous vertical neck bands at maximum contraction on a first side of the human patient's neck, wherein a continuous vertical neck band is a vertical neck band that extends continuously from the human patient's jawline to a lower neck region; and
(B) administering a composition comprising a botulinum toxin type A to the first side of the human patient's neck determined as having only one or only two continuous vertical neck bands at maximum contraction.

Embodiment 2: The method of embodiment 1, wherein the composition is administered to the first side of the human patient's neck by injection of:
(i) a first jawline dose divided among 4 injection sites in the upper segment of platysma muscle, inferior and parallel to the patient's lower mandibular border on the first side; and
(ii) (a) a first neck band dose divided among 5 injection sites along the only continuous vertical neck band if the first side of the human patient's neck has only one continuous vertical neck band; or (b) a first neck band dose divided among 5 injection sites along a first continuous vertical neck band and a second neck band dose divided among 5 injection sites along a second continuous vertical neck band if the first side of the human patient's neck has only two continuous vertical neck bands.

Embodiment 3: The method of embodiment 1 or 2, wherein the first side of the human patient's neck has only one continuous vertical neck band, and the administering step delivers a total unilateral dose of about 13 units of botulinum toxin type A to the first side of the human patient's neck.

Embodiment 4: The method of embodiment 1 or 2, wherein the first side of the human patient's neck has only two continuous vertical neck bands, and the administering step delivers a total unilateral dose of about 18 units of botulinum toxin type A to the first side of the human patient's neck.

Embodiment 5: The method of any one of embodiments 2-4, wherein:
(1) the first jawline dose is about 8 units of botulinum toxin type A and wherein optionally each injection of the first jawline dose is about 2 units of botulinum toxin type A;
(2) the first neck band dose is about 5 units of botulinum toxin type A and wherein optionally each injection of the first neck band dose is about 1 unit of botulinum toxin type A; and/or
(3) the second neck band dose is about 5 units of botulinum toxin type A and wherein optionally each injection of the second neck band dose is about 1 unit of botulinum toxin type A.

Embodiment 6: The method of any one of embodiments 1-5, further comprising repeating the steps of quantifying and administering to a second side of the human patient's neck.

Embodiment 7: The method of embodiment 6, wherein the step of administering to the first side and the step of administering to the second side deliver a total bilateral dose selected from the group consisting of about 26 units, about 31 units, and about 36 units of botulinum toxin type A to the human patient's neck.

Embodiment 8: A method for improving the appearance of platysma prominence associated with platysma muscle activity to a human patient who has only one continuous vertical neck band at maximum contraction on a first side of the neck, comprising administering a composition comprising a botulinum toxin type A to the first side of the human patient's neck, by injection of:
(i) a first jawline dose divided among 4 injection sites in the upper segment of platysma muscle, inferior and parallel to the patient's lower mandibular border on the first side; and
(ii) a first neck band dose divided among 5 injection sites along the only continuous vertical neck band;
wherein a continuous vertical neck band is a vertical neck band that extends continuously from the human patient's jawline to a lower neck region.

Embodiment 9: The method of embodiment 8, wherein the administering step delivers a total unilateral dose of about 13 units of botulinum toxin type A to the first side of the human patient's neck.

Embodiment 10: The method of embodiment 8 or 9, wherein:
(1) the first jawline dose is about 8 units of botulinum toxin type A and wherein optionally each injection of the first jawline dose is about 2 units of botulinum toxin type A; and
(2) the first neck band dose is about 5 units of botulinum toxin type A and wherein optionally each injection of the first neck band dose is about 1 unit of botulinum toxin type A.

Embodiment 11: The method of any one of embodiments 8-10, further comprising repeating the step of administering to a second side of the human patient's neck, wherein the second side of the human patient's neck has only one continuous vertical neck band at maximum contraction.

Embodiment 12: The method of embodiment 11, wherein the step of administering to the first side and the step of administering to the second side deliver a total bilateral dose of about 26 units of botulinum toxin type A to the human patient's neck.

Embodiment 13: A method for improving the appearance of platysma prominence associated with platysma muscle activity to a human patient who has only two continuous vertical neck bands at maximum contraction on a first side of the neck, comprising administering a composition comprising a botulinum toxin type A to the first side of the human patient's neck, by injection of:
(i) a first jawline dose divided among 4 injection sites in the upper segment of platysma muscle, inferior and parallel to the patient's lower mandibular border on the first side;
(ii) a first neck band dose divided among 5 injection sites along a first continuous vertical neck band; and
(iii) a second neck band dose divided among 5 injection sites along a second continuous vertical neck band;
wherein a continuous vertical neck band is a vertical neck band that extends continuously from the human patient's jawline to a lower neck region, and wherein the administering step delivers a total unilateral dose range of about 15 units to about 20 units of botulinum toxin type A to the first side of the human patient's neck.

Embodiment 14: The method of embodiment 13, wherein the administering step delivers a total unilateral dose of about 18 units of botulinum toxin type A to the first side of the human patient's neck.

Embodiment 15: The method of embodiment 13 or 14, wherein:
(1) the first jawline dose is about 8 units of botulinum toxin type A and wherein optionally each injection of the first jawline dose is about 2 units of botulinum toxin type A;
(2) the first neck band dose is about 5 units of botulinum toxin type A and wherein optionally each injection of the first neck band dose is about 1 unit of botulinum toxin type A; and
(3) the second neck band dose is about 5 units of botulinum toxin type A and wherein optionally each injection of the second neck band dose is about 1 unit of botulinum toxin type A.

Embodiment 16: The method of any one of embodiments 13-15, further comprising repeating the step of administering to a second side of the human patient's neck, wherein the second side of the human patient's neck has only two continuous vertical neck bands at maximum contraction.

Embodiment 17: The method of embodiment 16, wherein the step of administering to the first side and the step of administering to the second side deliver a total bilateral dose of about 36 units of botulinum toxin type A to the human patient's neck.

Embodiment 18: A method for improving the appearance of platysma prominence associated with platysma muscle activity to a human patient who has only one continuous vertical neck band at maximum contraction on a first side of the neck and only two continuous vertical neck bands at maximum contraction on a second side of the neck, the method comprising administering a composition comprising a botulinum toxin type A to the human patient's neck, by injection of:
(i) a first jawline dose divided among 4 injection sites in the upper segment of platysma muscle, inferior and parallel to the patient's lower mandibular border on the first side;
(ii) a first neck band dose divided among 5 injection sites along the only continuous vertical neck band on the first side;
(iii) a second jawline dose divided among 4 injection sites in the upper segment of platysma muscle, inferior and parallel to the patient's lower mandibular border on the second side;
(iv) a first neck band dose divided among 5 injection sites along a first continuous vertical neck band on the second side; and
(v) a second neck band dose divided among 5 injection sites along a second continuous vertical neck band on the second side;
wherein a continuous vertical neck band is a vertical neck band that extends continuously from the human patient's jawline to a lower neck region.

Embodiment 19: The method of embodiment 18, wherein the first jawline dose is equal to the second jawline dose.

Embodiment 20: The method of embodiment 18, wherein the first jawline dose is not equal to the second jawline dose.

Embodiment 21: The method of any one of embodiments 18-20, wherein:
(1) the first jawline dose is about 8 units of botulinum toxin type A and wherein optionally each injection of the first jawline dose is about 2 units of botulinum toxin type A;
(2) the second jawline dose is about 8 units of botulinum toxin type A and wherein optionally each injection of the second jawline dose is about 2 units of botulinum toxin type A;
(3) the first neck band dose on the first side is about 5 units of botulinum toxin type A and wherein optionally each injection of the first neck band dose on the first side is about 1 unit of botulinum toxin type A;
(4) the first neck band dose on the second side is about 5 units of botulinum toxin type A and wherein optionally each injection of the first neck band dose on the second side is about 1 unit of botulinum toxin type A; and
(5) the second neck band dose on the second side is about 5 units of botulinum toxin type A and wherein optionally each injection of the second neck band dose on the second side is about 1 unit of botulinum toxin type A.

Embodiment 22: The method of any one of embodiments 18-21, wherein the administering step delivers a total bilateral dose of about 31 units botulinum toxin type A to the human patient's neck.

Embodiment 23: The method of any one of embodiments 1-22, wherein a cosmetic effect of the method is obtained within 14 days following the administration of the botulinum toxin type A, and wherein:
(1) the cosmetic effect comprises a physician-assessed or human patient-assessed platysma prominence scale grade change from moderate or severe prior to the administration of the botulinum toxin type A to minimal or mild following the administration of the botulinum toxin type A; wherein optionally the duration of the cosmetic effect ranges from about 30 days to about 120 days following the administration of the botulinum toxin type A or the duration of the cosmetic effect is at least 120 days; and wherein optionally the method is statistically better than a comparable method wherein a placebo is administered instead of the botulinum toxin type A at achieving a physician-assessed or human patient-assessed grade of minimal or mild on a platysma prominence scale at 14 days post-administration;
(2) the cosmetic effect comprises a physician-assessed and human patient-assessed 2-grade or better improvement on a platysma prominence scale from moderate or severe prior to the administration of the botulinum toxin type A to minimal or mild following the administration of the botulinum toxin type A;
(3) the cosmetic effect comprises a physician-assessed and human patient-assessed platysma prominence scale grade change from moderate or severe prior to the administration of the botulinum toxin type A to minimal or mild following the administration of the botulinum toxin type A; or
(4) the cosmetic effect comprises achieving a physician-assessed and human patient-assessed grade of minimal or mild and at least a 2-grade improvement from baseline on a platysma prominence scale; wherein optionally the duration of the cosmetic effect is at least 120 days or the duration of the cosmetic effect ranges from about 60 days to about 120 days following the administration of the botulinum toxin type A; and wherein optionally the method is statistically better than a comparable method wherein a placebo is administered instead of the botulinum toxin type A at achieving both physician-assessed and human patient-assessed grade of minimal or mild and at least a 2-grade improvement from baseline on a platysma prominence scale at 14 days post-administration;
wherein optionally the platysma prominence scale is a platysma band severity grading scale.

Embodiment 24: The method of any one of embodiments 1-23, wherein the concentration of the botulinum toxin type A in the composition is about 40 units per mL.

Embodiment 25: The method of any one of embodiments 1-24, wherein the botulinum toxin type A is a 900 kD botulinum neurotoxin serotype A (BoNT/A) complex.

Embodiment 26: The method of embodiment 25, wherein the botulinum toxin type A is onabotulinumtoxinA.

Embodiment 27: The method of embodiment 25 or 26, wherein the composition comprises a powdered BoNT/A pharmaceutical composition that has been reconstituted with normal saline, wherein prior to the reconstitution, the powdered BoNT/A pharmaceutical composition consists of the 900 kDa BoNT/A complex, human albumin, and sodium chloride.

Embodiment 28: The method of any one of embodiments 1-27, wherein the composition is a liquid.

Embodiment 29: The method of any one of embodiments 2-28, wherein the injections are not made deeper than about 0.5 inches into a muscle; and/or wherein the injections are administered superficially and intramuscularly with the needle perpendicular to the surface of the skin.

Embodiment 30: The method of any one of embodiments 1-29, wherein the human patient further has one or more non-continuous neck bands on the first side and/or the second side of the human patient's neck; and/or wherein no additional dose of the botulinum toxin type A is injected to the human patient's neck.

Embodiment 31: The method of any one of embodiments 1-30, wherein the method is repeated about every 12 weeks.

Embodiment 32: The method of any one of embodiments 1-31, wherein the method results in at least one incidence rate selected from the group consisting of:
(1) an incidence rate of less than 5% of treatment-emergent adverse effects (TEAEs) related to the composition comprising the botulinum toxin type A;
(2) an incidence rate of less than 1% of treatment-related severe TEAEs, and optionally no incidence of treatment-related severe TEAE;
(3) an incidence rate of less than 5% of adverse events of special interest (AESIs), and optionally no incidence of AESIs, wherein AESIs include aspiration, aspiration pneumonia, dry mouth, dysphagia, dyspnea, facial paresis, and muscular weakness;
(4) an incidence rate of less than 10% of injection site bruising;
(5) an incidence rate of less than 1% of neck muscle weakness, and optionally no incidence of neck muscle weakness;
(6) an incidence rate of less than 2% of dysphagia, and optionally no incidence of dysphagia; and
(7) an incidence rate of less than 5% of injection site hematoma, and optionally no incidence of injection site hematoma.

Embodiment 33: The method of any one of embodiments 2-32, wherein the method results in a reduced incidence of adverse effects as compared to a comparable method that comprises:
(1) injecting one or more neck band doses of the botulinum toxin type A to one or more neck bands of a human subject's neck but does not comprise injecting a jawline dose of the botulinum toxin type A to the human subject's neck;
(2) injecting one or more neck band doses of the botulinum toxin type A to one or more neck bands of a human subject's neck, wherein the one or more neck bands injected with the one or more neck band doses are not limited to continuous vertical neck bands that extend continuously from the human subject's jawline to the lower neck region; and/or
(3) injecting three or more neck band doses of the botulinum toxin type A to one side of a human subject's neck, with each neck band dose being injected along a different neck band.

Embodiment 34: The method of embodiment 33, wherein the adverse effect is selected from the group consisting of aspiration, aspiration pneumonia, dry mouth, dysphagia, dyspnea, facial paresis, muscular weakness, injection site bruising, and injection site hematoma.

Embodiment 35: The method of any one of embodiments 1-34, which is for improving the appearance of platysma bands associated with platysma muscle activity.

Embodiment 36: The method of any one of embodiments 1-35, wherein a continuous vertical neck band is a visible vertical neck band that extends continuously from the human patient's jawline to a lower neck region and results in blunting of the jawline.

Embodiment 37: The method of any one of embodiments 2-36, wherein the 4 injection sites in the upper segment of platysma muscle comprise an anterior injection site in line with the oral commissure and a posterior injection site anterior to the angle of the mandible.

Embodiment 38: A composition comprising a botulinum toxin type A for use in the method of any one of embodiments 1-37.

Embodiment 39: Use of a composition comprising a botulinum toxin type A for the manufacturing of a medicament for providing improvement in the appearance of platysma prominence associated with platysma muscle activity to a human patient in accordance with the method of any one of embodiments 1-37.

Embodiment 40: Use of a composition comprising a botulinum toxin type A substantially as described herein.

Embodiment 41: A composition for the improvement of the appearance of platysma prominence substantially as described herein.

Embodiment 42: A method for the treatment of the appearance of platysma bands associated with platysma muscle activity in a human patient using a composition comprising a botulinum toxin type A, wherein the treatment comprises intramuscularly administering to a first side of the human patient's neck:
a) a first jawline dose divided among 4 injection sites in the upper segment of platysma muscle on the first side of the human patient's neck, wherein the first jawline dose is about 8 units of botulinum toxin type A; and
b) a first neck band dose divided among 5 injection sites along a first platysma band on the first side of the human patient's neck, wherein the first neck band dose is about 5 units of botulinum toxin type A;
and wherein the appearance of the platysma bands in the human patient is safely and effectively treated.

Embodiment 43: The method of embodiment 42, wherein the method further comprises intramuscularly administering to a second side of the patient's neck:
a) a second jawline dose divided among 4 injection sites in the upper segment of platysma muscle on the second side of the human patient's neck, wherein the second jawline dose is about 8 units of botulinum toxin type A; and
b) a second neck band dose divided among 5 injection sites along a first platysma band on the second side of the human patient's neck, wherein the second neck band dose is about 5 units of botulinum toxin type A.

Embodiment 44: The method of embodiment 43, wherein the method further comprises intramuscularly administering to at least one of the first or second side of the human patient's neck a third neck band dose divided among 5 injection sites along a third platysma band, wherein the third neck band dose is about 5 units of botulinum toxin type A.

Embodiment 45: The method of any one of embodiments 42-44, further comprising administering a fourth neck band dose divided among 5 injection sites along a fourth platysma band, wherein the fourth neck band dose is about 5 units of botulinum toxin type A, and wherein the human patient has two platysma bands located on each of the first and second sides of the human patient's neck.

Embodiment 46: The method of embodiment 43, wherein the administering step delivers a total bilateral dose of 26 units of botulinum toxin type A.

Embodiment 47: The method of embodiment 44, wherein the administering step delivers a total bilateral dose of 31 units of botulinum toxin type A.

Embodiment 48: The method of embodiment 44 or 45, wherein the administering step delivers a total bilateral dose of 36 units of botulinum toxin type A.

Embodiment 49: The method of any one of embodiments 42-48, wherein the first platysma band on the first side, the first platysma band on the second side, the third platysma band on the at least one of the first or second side of the human patient's neck, and/or the fourth platysma band are visible continuous vertical neck bands at maximum contraction that extend continuously from the human patient's jawline to a lower neck region and result in blunting of the jawline.

Embodiment 50: The method of any one of embodiments 42-49, wherein the 4 jawline injection sites are administered inferior and parallel to the patient's lower mandibular border.

Embodiment 51: The method of embodiment 50, wherein the 4 jawline injection sites are administered approximately 1-2 cm inferior and parallel to the patient's lower mandibular border.

Embodiment 52: The method of embodiment 50 or 51, wherein the first jawline injection site is an anterior injection site in line with the patient's oral commissure, the fourth jawline injection site is a posterior injection site anterior to the angle of the mandible, and the second and third jawline injection sites are equidistant between the first and fourth jawline injection sites.

Embodiment 53: The method of any one of embodiments 42-52, wherein the first injection site of the first, second, third or fourth neck band dose is administered approximately 1-2 cm inferior to the jawline injection sites.

Embodiment 54: The method of embodiment 53, wherein the neck band injection sites are spaced approximately 1-2 cm apart.

Embodiment 55: The method of any one of embodiments 42-54, wherein the treatment provides temporary improvement in the appearance of the platysma bands.

### 7. EXAMPLES

Certain embodiments provided herein are illustrated by the following non-limiting examples, which describe a holistic approach for treating platysma prominence using a botulinum toxin and which show that the holistic approach has a favorable benefit/risk profile.

### 7.1. Example 1: Use of a Botulinum Toxin Type A (BOTOX®) for the Treatment of Platysma Prominence

A multicenter, randomized, double-blind, placebo-controlled, dose-ranging Phase 2 study was carried out to evaluate the safety and efficacy of a botulinum toxin type A purified neurotoxin complex (BOTOX®) for the treatment of platysma prominence.

The purpose of this placebo-controlled Phase 2 trial was to evaluate the safety and efficacy of a high and a low dose of BOTOX® compared with placebo to reduce the appearance of platysma prominence in adult participants with moderate to severe platysma prominence. The overall duration of the study was 4 months with 171 enrolled participants. Participants were screened from Day −14 to Day −7 (screening period). Participants were men and women over 18 years old who had either moderate (Grade 3) or severe (Grade 4) platysma prominence on both left and right sides (severity does not have to be identical on both sides), as determined at maximum contraction at screening by the investigator using C-APPS and on Day 1 by the investigator and the participant using the C-APPS and P-APPS, respectively (see FIG. 1 and Table 3). Exclusion criteria included certain medical conditions (including dysphagia, pregnancy, and conditions that may put the participant at increased medical risk with exposure to BOTOX), and prior exposure to botulinum toxin within 6 months prior to Day 1. Follow-up visits were scheduled for Days 7, 14, 30, 60, and 90, and study exit (Day 120).

After the screening period, on Day 1 participants who satisfied the inclusion criteria were randomized in a 1:1:1 allocation ratio to receive:
BOTOX high dose (52 U, 62 U, or 72 U, depending on baseline (Day 1) C-APPS rating assessed by the investigator)
BOTOX low dose (26 U, 31 U, or 36 U, depending on baseline (Day 1) C-APPS rating assessed by the investigator)
Placebo (0 U)

For each participant, superficial intramuscular injections were administered to the upper segment of the platysma muscle below the jawline (0.1 mL per injection, approximately 1 cm apart) and along each continuous vertical neck band on each side of the neck (up to 2 bands per side, 0.05 mL per injection, approximately 1-2 cm apart). Dosing was determined based on baseline investigator C-APPS score on Day 1 (FIGS. 2A-2B and Table 2):
A participant with Grade 3 platysma prominence on both sides received a total of 18 injections (9 injections on each side: 4 injections in the upper segment of the platysma muscle below the jawline and 5 injections along the continuous vertical neck band, on each side).
A participant with Grade 3 platysma prominence on 1 side and Grade 4 platysma prominence on the other side received a total of 23 injections (9 injections on the side with Grade 3 platysma prominence, and 14 injections on the side with Grade 4 platysma prominence, as described above).
A participant with Grade 4 platysma prominence on both sides received a total of 28 injections (14 injections on each side: 4 injections in the upper segment of the platysma muscle below the jawline, 5 injections along the anterior vertical neck band, and 5 injections along the posterior vertical neck band, on each side).

To illustrate, a participant with Grade 4 platysma prominence on both sides randomized to BOTOX high dose would receive a total dose of 28 injections or 72 U, as follows:
Dose administered to the upper segment of the platysma muscle below the jawline=(2 sides)×(4 injections under each jawline)×(4 U)=32 U
Dose administered to continuous vertical neck bands=(2 sides)×(2 vertical neck bands per side)×(5 injections per vertical neck band)×(2 U)=40 U

TABLE 2
Number of Injection and Total Dose.

| Baseline | Total | Total Dose on Day 1 | | |
|---|---|---|---|---|
| investigator | Number of | BOTOX | BOTOX | |
| assessed-C-APPS | Injections on | High | Low | |
| scores on Day 1 | Day 1 | Dose | Dose | Placebo |
| Grade 3 on both sides | 18 injections | 52 U | 26 U | 0 U |
| Grade 3 on one side | 23 injections | 62 U | 31 U | 0 U |
| Grade 4 on other side | | | | |
| Grade 4 on both sides | 28 injections | 72 U | 36 U | 0 U |

In practicing this example, medical practitioners were instructed in accordance with the following: each 100 unit vial of onabotulinumtoxinA (BOTOX®) was diluted with 2.5 mL or 5 mL of sterile, preservative-free saline to achieve 2 U/0.05 mL or 1 U/0.05 mL for the BOTOX high dose, or BOTOX low dose, respectively, and each vial of placebo was diluted with 2.5 mL of sterile, preservative-free saline.

The primary efficacy endpoint included an achievement of at least a 1-grade improvement on both the left and right sides at Day 14, as rated at maximum contraction by investigator using the APPS. The secondary efficacy endpoint included an achievement of at least a 1-grade improvement on both the left and right sides at Day 14, as rated at maximum contraction by participant using the APPS.

Platysma prominence on the left and right sides was assessed independently at maximum contraction by the investigator or participant using the C-APPS and P-APPS respectively (1=minimal, 2=mild, 3=moderate, 4=severe, 5=extreme; Table 3, FIG. 1).

TABLE 3
Allergan Platysma Prominence Scale (APPS) at Maximum Contraction
(Grades and Descriptions) (illustrated in FIG. 1).

| Grade | Term | Description |
|---|---|---|
| 1 | Minimal | No bands or small disjointed bands |
| 2 | Mild | Disjointed bands |
| 3 | Moderate | 1 continuous band |
| 4 | Severe | 2 continuous bands |
| 5 | Extreme | 3 or more prominent bands |

Any adverse events (AEs) were reported by the participant (or, when appropriate, by a caregiver or surrogate) and assessed according to the classification shown in Table 4.

TABLE 4
Assessment of Intensity of AE.

| | |
|---|---|
| MILD | A type of AE that is usually transient and |
| | may require only minimal treatment |
| | or therapeutic intervention. The event does |
| | not generally interfere with usual |
| | activities of daily living. |
| MODERATE | A type of AE that is usually alleviated with |
| | additional specific therapeutic intervention. |
| | The event interferes with usual activities |
| | of daily living, causing discomfort but |
| | poses no significant or permanent risk of |
| | harm to the research participant. |
| SEVERE | A type of AE that interrupts usual activities |
| | of daily living, or significantly affects |
| | clinical status, or may require intensive |
| | therapeutic intervention. |

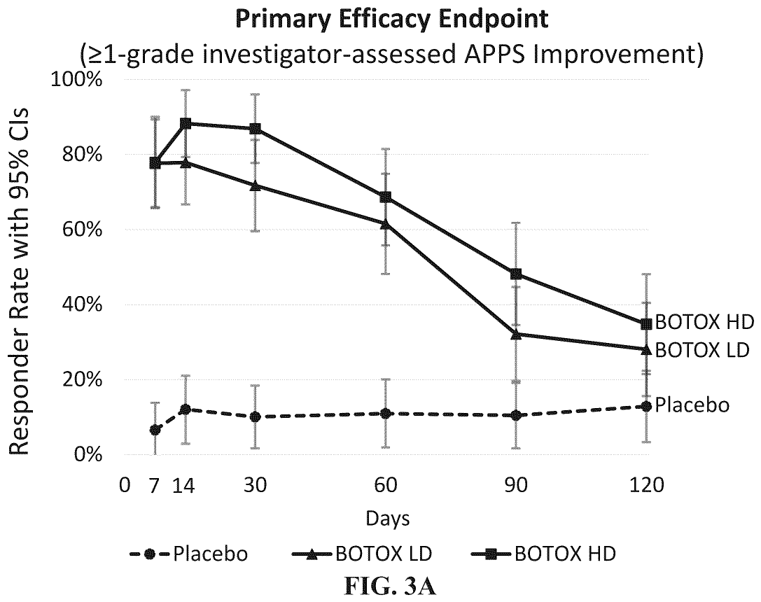

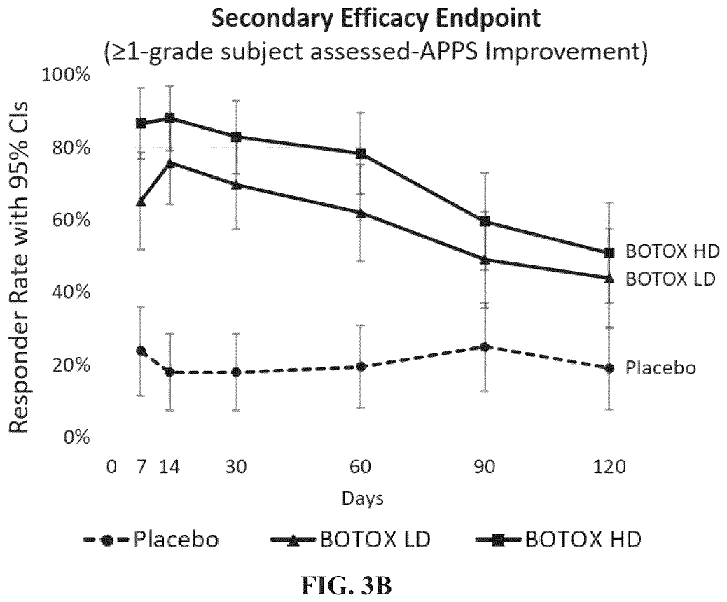

Data for the primary and secondary efficacy endpoints are shown in FIGS. 3A and 3B, respectively. FIG. 3A shows that both the BOTOX high dose group and the BOTOX low dose group had statistically higher responder rate than the placebo group at achieving at least a 1-grade improvement on both the left and right sides at Day 14 (p-value<0.0001), as rated at maximum contraction by investigator using the C-APPS (i.e., the primary efficacy endpoint). FIG. 3B shows that both the BOTOX high dose group and the BOTOX low dose group had statistically higher responder rate than the placebo group at achieving at least a 1-grade improvement on both the left and right sides at Day 14 (p-value<0.0001), as rated at maximum contraction by participant using the P-APPS (i.e., the secondary efficacy endpoint). Therefore, the data show that both primary and secondary endpoints were achieved for both the BOTOX high dose and low dose groups.

The safety data are summarized in Table 5 below. The majority of treatment-related adverse events (AEs) were mild in severity. Incidence of treatment-related AEs was much higher in the BOTOX high dose group. The majority of the study drug-related treatment-emergent adverse events (TEAEs) were in the BOTOX high dose group (an AE will be considered a TEAE if the AE began after the first dose of study intervention, or the AE was present before the first dose of study intervention, but increased in severity or became serious after the first dose of study intervention). In addition, the reported treatment-related AEs were not unexpected. All of them were consistent with known BOTOX pharmacological effects and literature findings.

TABLE 5
Phase 2 Safety Data Summary.

| | | BOTOX | BOTOX |
|---|---|---|---|
| | Placebo | Low Dose | High Dose |
| System Organ Class | (N = 56) | (N = 59) | (N = 54) |
| Preferred Term | n (%) | n (%) | n (%) |
| Participants with at least one study | 0 (0.0) | 1 (1.7) | 9 (16.7) |
| drug-related TEAE | | | |
| Gastrointestinal disorders | 0 (0.0) | 0 (0.0) | 2 (3.7) |
| Dysphagia | 0 (0.0) | 0 (0.0) | 2 (3.7) |
| Musculoskeletal and connective | 0 (0.0) | 0 (0.0) | 7 (13.0) |
| tissue disorders | | | |
| Muscular weakness | 0 (0.0) | 0 (0.0) | 5 (9.3) |
| Muscle spasms | 0 (0.0) | 0 (0.0) | 1 (1.9) |
| Musculoskeletal stiffness | 0 (0.0) | 0 (0.0) | 1 (1.9) |
| Myalgia | 0 (0.0) | 0 (0.0) | 1 (1.9) |
| Neck pain | 0 (0.0) | 0 (0.0) | 1 (1.9) |
| Nervous system disorders | 0 (0.0) | 1 (1.7) | 1 (1.9) |
| Facial paresis | 0 (0.0) | 1 (1.7) | 1 (1.9) |

In conclusion, the results of this Phase 2 study show that BOTOX treatment of platysma prominence was more effective than placebo (as determined by clinicians and participants using the APPS) and that the treatment had a desirable safety profile after a single treatment at doses ranging from 26 U to 36 U.

### 7.2. Example 2: Use of a Botulinum Toxin Type A (BOTOX®) for the Treatment of Platysma Prominence

Two phase 3 multicenter, randomized, double-blind, placebo-controlled studies were carried out to evaluate the safety and efficacy of a botulinum toxin type A purified neurotoxin complex (BOTOX®) for the improvement in the appearance of moderate to severe platysma prominence. The first clinical study (study 1) is also referred to as the M21-309 study and the second clinical study (or study 2) is also referred to as the M21-310 study.

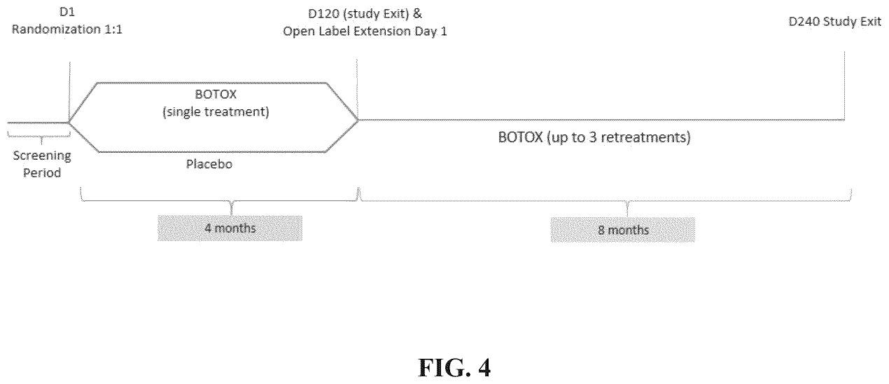

Based on left- and right-side severity, subjects were randomized to receive a single treatment of BOTOX® (26 Units, 31 Units, or 36 Units) or placebo. The overall duration of each of the two studies was 4 months. Participants were screened from Day −14 to Day −7 (screening period). Participants were men and women at least 18 years old who had either moderate (Grade 3) or severe (Grade 4) platysma prominence on both the left and right sides as determined at maximum contraction at screening and on Day 1 independently by the investigator using the 5-grade Clinician Allergan Platysma Prominence Scale (C-APPS) and by the participant using the 5-grade Participant Allergan Platysma Prominence Scale (P-APPS) (see FIG. 1 and Table 3). The severities of platysma prominence on the left and right sides did not have to be identical. Exclusion criteria included certain medical conditions (including dysphagia, pregnancy, and conditions that may put the participant at increased medical risk with exposure to BOTOX®), and prior exposure to botulinum toxin within 6 months prior to Day 1. Follow-up visits were scheduled for Days 14, 30, 60, and 90, and study exit (Day 120). Upon exit from study 1, eligible subjects had the option to participate in an 8-month open-label extension study (see Example 3 and FIG. 4) to further assess long term safety and efficacy.

After the screening period, eligible subjects were randomized on Day 1 in a 1:1 ratio to receive BOTOX® or placebo.

For each subject, superficial intramuscular injections were administered to the upper segment of the platysma muscle, below the jawline on each side (0.05 mL per injection, approximately 1 cm apart) and along each continuous vertical neck band on each side of the neck (up to 2 bands per side, 0.025 mL per injection, approximately 1 to 2 cm apart). Dosing was determined based on the baseline investigator assessed-C-APPS score on Day 1. Depending on platysma prominence severity, the total dose may be 26 Units (1 band/side), 31 Units (1 band one side, 2 bands other side), or 36 Units (2 bands/side) (Tables 6A-6B and FIGS. 2A-2B):
A subject with Grade 3 platysma prominence on both sides received a total of 26 U or 18 injections (9 injections on each side: 4 injections in the upper segment of the platysma muscle below the jawline and 5 injections along the continuous vertical neck band, on each side)
A subject with Grade 3 platysma prominence on 1 side and Grade 4 platysma prominence on the other side received a total of 31 U or 23 injections (9 injections on the side with Grade 3 platysma prominence, and 14 injections on the side with Grade 4 platysma prominence)
A subject with Grade 4 platysma prominence on both sides received a total of 36 U or 28 injections (14 injections on each side: 4 injections in the upper segment of the platysma muscle below the jawline, 5 injections along the anterior vertical neck band, and 5 injections along the posterior vertical neck band, on each side)

To illustrate, a subject with Grade 4 platysma prominence on both sides randomized to receive BOTOX would receive a total dose of 28 injections or 36 U, as follows:
Dose administered to the upper segment of the platysma muscle below the jawline=(2 sides)×(4 injections under each jawline)×(2 U)=16 U
Dose administered to continuous vertical neck bands=(2 sides)×(2 vertical neck bands per side)×(5 injections per vertical neck band)×(1 U)=20 U

TABLE 6A
Number of Injections and Total Dose.

| Baseline Investigator | Total Number | | |
|---|---|---|---|
| Assessed-C-APPS | of Injections | Total Dose on Day 1 | |
| Scores on Day 1 | on Day 1 | BOTOX | Placebo |
| Grade 3 on both sides | 18 injections | 26 U | 0 U |
| Grade 3 on one side/ | 23 injections | 31 U | 0 U |
| Grade 4 on other side | | | |
| Grade 4 on both sides | 28 injections | 36 U | 0 U |

TABLE 6B
Total Dose Distribution

| Jawline Injection | | | Total Dose |
|---|---|---|---|
| (Inferior to the | | | (Number of |
| Lower Mandibular | | | Injection |
| Border) | Vertical Neck Band Injection | | Sites) |
| | | 1 band on both sides | 26 Units |
| | | (10 Units in 10 sites) | (18 sites) |
| 2 Units/0.05 mL into | 1 Unit/0.025 mL | 1 band on one side, and 2 | 31 Units |
| 4 sites on each side | into 5 sites per band | bands on the other side | (23 sites) |
| (16 Units in 8 sites) | (1 to 2 bands/side) | (15 Units in 15 sites) | |
| | | 2 bands on both sides | 36 Units |
| | | (20 Units in 20 sites) | (28 sites) |

In practicing this example, medical practitioners were instructed in accordance with the following:

Dilute each 100 unit vial of onabotulinumtoxinA (BOTOX®, BOTOX® Cosmetic or VISTABEL®) with 2.5 mL of sterile, preservative-free saline to achieve 4 units/0.1 mL for the BOTOX®, and dilute each vial of placebo with 2.5 mL of sterile, preservative-free saline.

Using an appropriately sized sterile syringe, needle, and aseptic technique, inject 2 Units/0.05 mL of reconstituted BOTOX® into 4 sites in the upper segment of platysma muscle, below the jawline on each side. In addition, inject 1 Unit/0.025 mL of reconstituted BOTOX® into 5 sites along each vertical neck band, 1 to 2 vertical neck bands per side. Depending on platysma prominence severity, the total dose may be 26 Units (1 band/side), 31 Units (1 band one side, 2 bands other side), or 36 Units (2 bands/side) (FIGS. 2A and 2B and Table 6B).

For each side, the 4 jawline injections to the upper platysma muscle should be approximately 1 to 2 cm inferior and parallel to the lower mandibular border. The anterior injection site should be in line with the oral commissure, and the posterior injection site should be slightly anterior to the angle of the mandible. The remaining two injections should be equidistant (approximately 1 to 2 cm apart) between the anterior and posterior injection points (FIGS. 2A and 2B). For each vertical neck band identified, 1 to 2 per side (FIGS. 2A and 2B), distribute 5 injections vertically approximately 1 to 2 cm apart. The most superior injection site should be approximately 1 to 2 cm inferior to the jawline injections.

The platysma muscle is a thin muscle sheet just below the surface of the skin. Therefore, all platysma muscle injections should be administered superficially and intramuscularly with the needle perpendicular to the surface of the skin. For vertical neck band injections, each band should be identified while the patient is contracting their platysma. Gently pinch the band to isolate the muscle from nearby anatomical structures during administration.

To reduce injection-related complications, injection should be at least 1 cm inferior to the lower mandibular border. Do not inject into structures deep to the platysma muscle, particularly in the anterior region of the neck.

For the US regulatory agency (i.e., FDA), the composite primary efficacy endpoint was achievement of a Grade 1 or 2 (Minimal or Mild) and at least a 2-grade improvement from baseline based on both investigator's assessment using the C-APPS and subject's self-assessment using the P-APPS at maximum contraction at Day 14.

For EU regulatory agencies, the coprimary efficacy endpoints were the achievement of at least a 2-grade improvement from baseline based on the following: (1) investigator's assessment using the C-APPS at maximum contraction at Day 14, and (2) subject's self-assessment using the P-APPS at maximum contraction at Day 14.

Secondary endpoints included:
Achievement of Grade 1 or 2 (Minimal or Mild) according to investigator's assessment using the C-APPS at maximum contraction over time
Achievement of Grade 1 or 2 (Minimal or Mild) according to subject's self-assessment using the P-APPS at maximum contraction over time
Responses of Satisfied or Very satisfied on the Appearance of Neck and Lower Face Questionnaire (ANLFQ): Satisfaction (Follow-up) Item 5 at Day 14. The responses to Item 5 range from Very Satisfied to Very Dissatisfied.
Responses of Not at all bothered or A little bothered on the Bother Assessment Scale
   Platysma Prominence (BAS-PP) Item 2 (jawline) at Day 14.
Responses of Not at all bothered or A little bothered on the BAS-PP Item 1 (vertical neck bands) at Day 14
Change from baseline on the ANLFQ: Impacts summary score at Day 14
Achievement of at least a 1-grade improvement from baseline based on investigator's assessment using the C-APPS at maximum contraction over time
Achievement of at least a 1-grade improvement from baseline based on subject's self-assessment using the P-APPS at maximum contraction over time

The investigators monitored each subject for clinical evidence of adverse events (AEs) on a routine basis throughout the study and assessed AEs according to the classification shown in Table 7. In both the M21-309 and the M21-310 studies, adverse events of special interest (AESIs) which were monitored included aspiration, aspiration pneumonia, dry mouth, dysphagia, dyspnea, facial paresis, and muscular weakness (i.e., neck muscle weakness).

TABLE 7
Severity of AE.

| | |
|---|---|
| Mild | The AE is transient and easily |
| | tolerated by the subject. |
| Moderate | The AE causes the subject discomfort and |
| | interrupts the subject's usual activities. |
| Severe | The AE causes considerable interference with |
| | the subject's usual activities and may be |
| | incapacitating or life threatening. |

There was no discontinuation due to deaths, adverse events (AEs), or COVID-19.

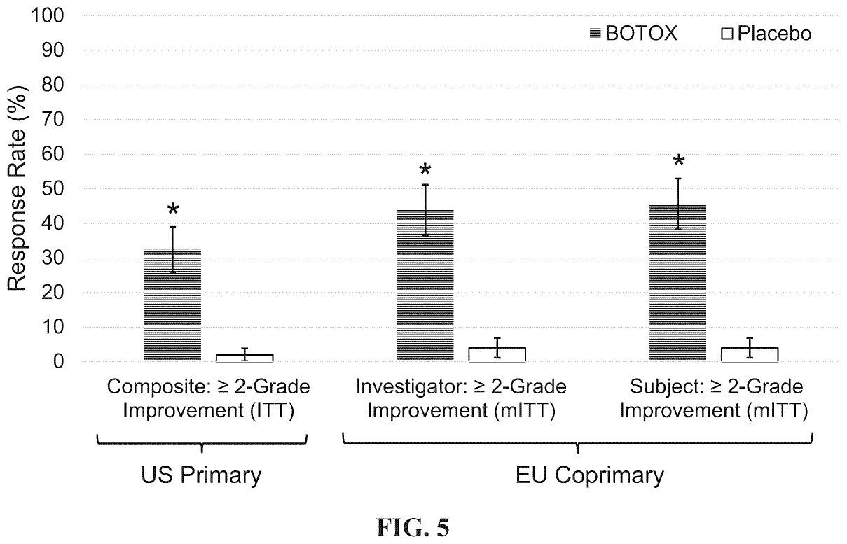

The primary efficacy endpoint was defined as composite achievement of Grade 1 or 2 (Minimal or Mild) and at least a 2-grade improvement from baseline in platysma prominence severity at maximum contraction, assessed by both investigator and subject on a per-subject basis at Day 14. The proportion of responders was greater in the BOTOX® ˜group compared to placebo at Day 14 (p<0.0001 for both studies) as shown in Table 8 and FIGS. 5, 6A, 8A and B.

TABLE 8
Studies 1 and 2: Number and Percentage of Subjects Achieving a Grade 1 or
2 (Minimal or Mild) and ≥2-Grade Improvement from Baseline Based on Investigator and
Subject Assessments of Platysma Prominence Severity at Maximum Contraction at Day 14.

| | Study 1 | | | Study 2 | | |
|---|---|---|---|---|---|---|
| | BOTOX ® | | | BOTOX ® | | |
| | 26, 31, or 36 | | | 26, 31, or 36 | | |
| | Units | Placebo | | Units | Placebo | |
| | (N = 199) | (N = 209) | | (N = 209) | (N = 217) | |
| | n (%) | n (%) | p-value | n (%) | n (%) | p-value |
| ≥ 2-Grade | 64 (32.3%) | 4 (1.9%) | p < 0.0001 | 66 (31.4%) | 1 (0.5%) | p < 0.0001 |
| Composite^{a} | | | | | | |

Minimal or Mild

| | | | | | | |
|---|---|---|---|---|---|---|
| Investigator | 113 (56.9%) | 12 (5.8%) | — | 101 (48.3%) | 7 (3.0%) | — |
| Assessment | | | | | | |
| Subject | 103 (51.7%) | 11 (5.1%) | — | 99 (47.1%) | 11 (5.1%) | — |
| Assessment | | | | | | |

^{a}Composite achievement of ≥2-grade improvement from baseline based on both the investigator and subject assessments and achievement of Grade 1 or 2 at Day 14.

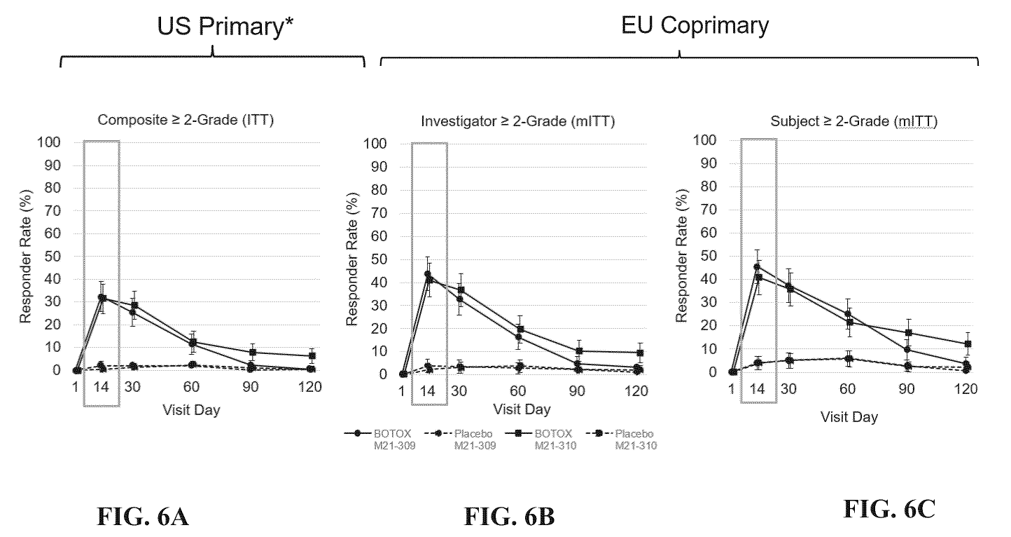

Efficacy results obtained in the first study show that the BOTOX® ˜treatment significantly reduced the severity of platysma prominence (see Table 8, FIG. 5). For the first study, response rates at the primary timepoint Day 14 were: (1) 32.3% and 1.90/for BOTOX and placebo, respectively, for the US primary endpoint (i.e., achievement of a Grade 1 or 2 (Minimal or Mild) and at least a 2-grade improvement from baseline based on both investigator's assessment using the C-APPS and subject's self-assessment using the P-APPS at maximum contraction at Day 14) in the ITT population; (2) 43.8% and 3.9% for BOTOX® and placebo, respectively, for the investigator assessment-based EU coprimary endpoint (i.e., ≥2-grade improvement from baseline based on investigator's assessment using the C-APPS at maximum contraction at Day 14) in the mITT population; and (3) 45.6% and 3.9% for BOTOX® and placebo, respectively, for the subject assessment-based EU coprimary endpoint (i.e., ≥2-grade improvement from baseline based on subject's assessment using the P-APPS at maximum contraction at Day 14) in the mITT population. Comparable efficacy results were also obtained in the second study (see Table 8 and FIGS. 6A-6C).

Primary measures demonstrated peak efficacy around Day 14 (see FIGS. 6A-6C). FIG. 6A shows the response rate for achieving the US primary endpoint in the ITT populations of the M21-309 and M21-310 studies, respectively (i.e., achievement of a Grade 1 or 2 (Minimal or Mild) and at least a 2-grade improvement from baseline based on both investigator's assessment using the C-APPS and subject's self-assessment using the P-APPS at maximum contraction at Day 14). FIGS. 6B and 6C show the response rate for achieving the EU coprimary endpoints in the mITT populations of the M21-309 and M21-310 studies, respectively (i.e., achievement of at least a 2-grade improvement from baseline based on investigator's assessment using the C-APPS at maximum contraction at Day 14 (FIG. 6B), and achievement of at least a 2-grade improvement from baseline based on subject's self-assessment using the P-APPS at maximum contraction at Day 14 (FIG. 6C)).

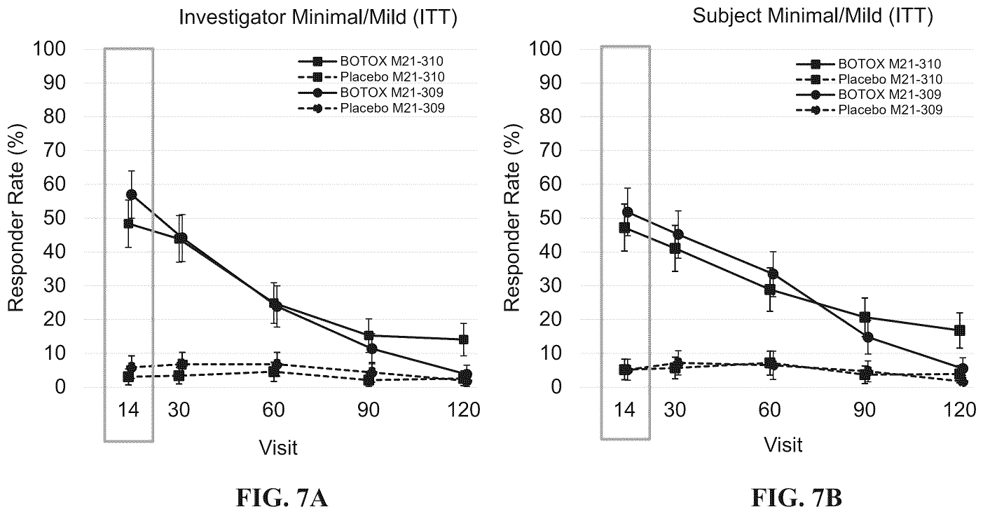

FIG. 7A shows that the response rates for achieving an improvement to Grade 1 or 2 (Minimal or Mild) based on investigator's assessment using the C-APPS at maximum contraction at Day 14 in the ITT population of the M21-309 study were 56.9% and 5.8% for BOTOX® and placebo, respectively (p-value<0.0001, unadjusted), and of the M21-310 study were 48.3% and 3.0% for BOTOX® and placebo, respectively (p-value<0.0001). FIG. 7B shows that the response rates for achieving an improvement to Grade 1 or 2 (Minimal or Mild) based on subject's assessment using the P-APPS at maximum contraction at Day 14 in the ITT population of the M21-309 study were 51.7% and 5.1% for BOTOX® and placebo, respectively (p-value<0.0001, unadjusted), and of the M21-310 study were 47.1% and 5.1% for BOTOX® and placebo, respectively (p-value<0.0001).

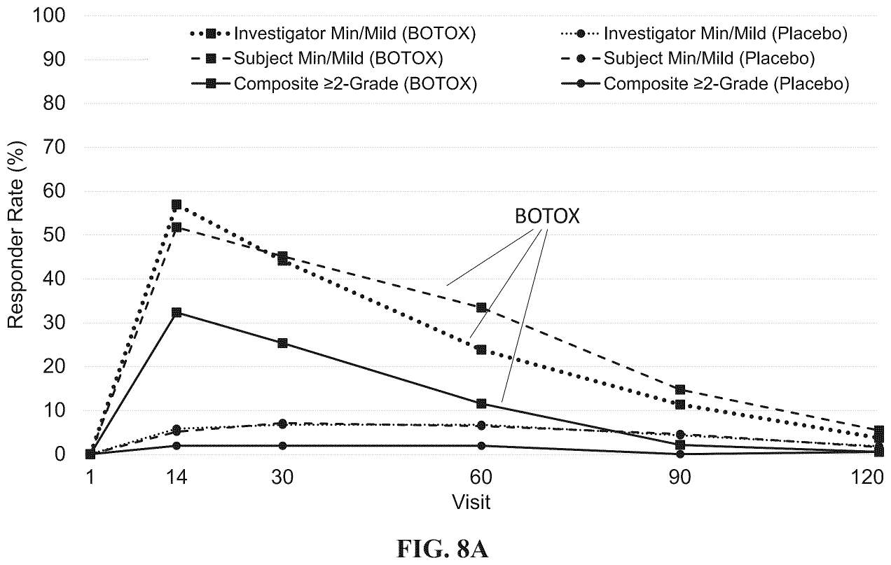

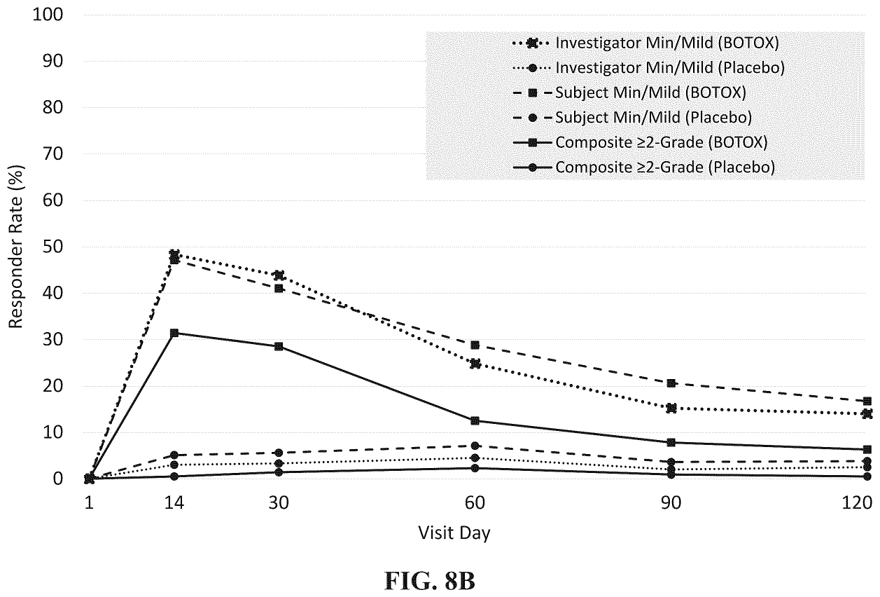

FIG. 8A shows that the responder rate (%) of participants in the ITT population of the M21-309 study who achieved: (i) an improvement to Grade 1 or 2 (Minimal or Mild) based on investigator's assessment using the C-APPS at maximum contraction at Day 14 were 56.9% and 5.8% for BOTOX® and placebo, respectively (p-value<0.0001, unadjusted); (ii) an improvement to Grade 1 or 2 (Minimal or Mild) based on subject's assessment using the P-APPS at maximum contraction at Day 14 were 51.7% and 5.1% for BOTOX® and placebo, respectively (p-value<0.0001, unadjusted); and (iii) at least a 2-grade improvement based on both investigator's assessment using the C-APPS and subject's self-assessment using the P-APPS at Day 14 were 32.3% and 1.9% for BOTOX® and placebo, respectively (p-value<0.0001). FIG. 8B shows that the responder rate (%) of participants in the ITT population of the M21-310 study who achieved: (i) an improvement to Grade 1 or 2 (Minimal or Mild) based on investigator's assessment using the C-APPS at maximum contraction at Day 14 were 48.3% and 3.0% for BOTOX® and placebo, respectively (p-value<0.0001); (ii) an improvement to Grade 1 or 2 (Minimal or Mild) based on subject's assessment using the P-APPS at maximum contraction at Day 14 were 47.1% and 5.1% for BOTOX® and placebo, respectively (p-value<0.0001); and (iii) at least a 2-grade improvement based on both investigator's assessment using the C-APPS and subject's self-assessment using the P-APPS at Day 14 were 31.4% and 0.0% for BOTOX® and placebo, respectively (p-value<0.0001).

The BOTOX® treatment demonstrated statistically higher response rate than placebo for the US primary endpoint (i.e., achievement of a Grade 1 or 2 (Minimal or Mild) and at least a 2-grade improvement from baseline based on both investigator's assessment and subject's self-assessment using the C-APPS and P-APPS respectively at maximum contraction at Day 14) (see FIGS. 5, 6A, 8A-8B and Table 8). The BOTOX® treatment also demonstrated higher response rate than placebo at achieving Grade 1 or 2 (Minimal or Mild) based on investigator's assessment using the C-APPS at maximum contraction at Day 14 (see FIGS. 7A, 8A-8B). The BOTOX® treatment also demonstrated higher response rate than placebo at achieving Grade 1 or 2 (Minimal or Mild) based on subject's assessment using the P-APPS at maximum contraction at Day 14 (see FIGS. 7B, 8A-8B).

Secondary efficacy measures included the Appearance of Neck and Lower Face Questionnaire (ANLFQ): Satisfaction (Follow-up) and Bother Assessment Scale-Platysma Prominence (BAS-PP).

Subjects assessed their satisfaction with the effect of treatment using ANLFQ: Satisfaction (Follow-up) Item #5 at Day 14, with a 5-point response scale (1=Very satisfied, 2=Satisfied, 3=Neither satisfied nor dissatisfied, 4=Dissatisfied, 5=Very dissatisfied).

Subjects assessed how bothered they were with the appearance of their platysma prominence using BAS-PP. Specifically, subjects assessed how bothered they were with the appearance of their vertical neck bands (BAS-PP Item #1) and the jawline (BAS-PP Item #2) at Day 14, using a 5-point response scale (1=Not at all bothered, 2=A little bothered, 3=Somewhat bothered, 4=A lot bothered, 5=Extremely bothered).

The results for secondary efficacy endpoints are presented for Studies 1 and 2 in Table 9A and Table 9B.

TABLE 9A
Studies 1 and 2: Results^{a} for Appearance of Neck and Lower Face Questionnaire:
Satisfaction (Follow-up) Item 5 (Satisfaction with Effect of Treatment)
at Day 14 (Number and Percentage of Subjects).

| | Study 1 | | Study 2 | |
|---|---|---|---|---|
| | BOTOX ® | | BOTOX ® | |
| | 26, 31, or 36 | | 26, 31, or | |
| | Units | Placebo | 36 Units | Placebo |
| | (N = 199) | (N = 209) | (N = 209) | (N = 217) |
| Responses | n (%) | n (%) | n (%) | n (%) |
| “Very satisfied” | 62 (31.2%) | 5 (2.4%) | 45 (21.5%) | 3 (1.4%) |
| “Satisfied” | 70 (35.2%) | 16 (7.7%) | 87 (41.6%) | 22 (10.1%) |
| “Neither satisfied nor dissatisfied” | 49 (24.6%) | 66 (31.6%) | 51 (24.4%) | 71 (32.7%) |
| “Dissatisfied” | 14 (7.0%) | 73 (34.9%) | 21 (10.1%) | 64 (29.5%) |
| “Very dissatisfied” | 4 (2.0%) | 49 (23.4%) | 5 (2.4%) | 57 (26.3%) |

^{a}Secondary endpoint response rate of “Very satisfied” or “Satisfied” was higher for the BOTOX ® group compared to placebo group at Day 14 (p < 0.0001)

TABLE 9B
Studies 1 and 2: Results^{a} for Bother Assessment Scale - Platysma
Prominence Item 1 (Vertical Neck Bands) and Item 2 (Jawline)
at Day 14 (Number and Percentage of Subjects).

| | Item 1 (Vertical Neck Bands) | | Item 2 (Jawline) | |
|---|---|---|---|---|
| | Study 1 | | | |
| | BOTOX ® | | BOTOX ® | |
| | 26, 31, or 36 | | 26, 31, or 36 | |
| | Units | Placebo | Units | Placebo |
| Responses | (N = 199) | (N = 209) | (N = 199) | (N = 209) |
| Bothered | n (%) | n (%) | n (%) | n (%) |
| “Not at all” | 48 (24.1%) | 7 (3.4%) | 45 (22.6%) | 9 (4.3%) |
| “A little” | 56 (28.1%) | 7 (3.4%) | 63 (31.7%) | 20 (9.6%) |
| “Somewhat” | 58 (29.2%) | 75 (35.9%) | 50 (25.1%) | 70 (33.5%) |
| “A lot” | 26 (13.1%) | 87 (41.6%) | 36 (18.1%) | 79 (37.8%) |
| “Extremely” | 11 (5.5%) | 33 (15.8%) | 5 (2.5%) | 31 (14.8%) |

| | Study 2 | | | |
|---|---|---|---|---|
| | BOTOX ® | | BOTOX ® | |
| | 26, 31, or 36 | | 26, 31, or 36 | |
| | Units | Placebo | Units | Placebo |
| | (N = 209) | (N = 217) | (N = 209) | (N = 217) |
| Bothered | n (%) | n (%) | n (%) | n (%) |
| “Not at all” | 43 (20.6%) | 6 (2.8%) | 34 (16.3%) | 17 (7.8%) |
| “A little” | 63 (30.1%) | 23 (10.6%) | 70 (33.5%) | 30 (13.8%) |
| “Somewhat” | 66 (31.6%) | 89 (41.0%) | 55 (26.3%) | 77 (35.5%) |
| “A lot” | 28 (13.4%) | 68 (31.3%) | 37 (17.7%) | 69 (31.8%) |
| “Extremely” | 9 (4.3%) | 31 (14.3%) | 13 (6.2%) | 24 (11.1%) |

^{a}Secondary endpoint response rates for Item 1 (vertical neck bands) and for Item 2 (jawline, specifically, how defined jawline looks) for “Not at all bothered” or “A little bothered” were higher for the BOTOX ® group compared to placebo group (p < 0.0001 for each item)

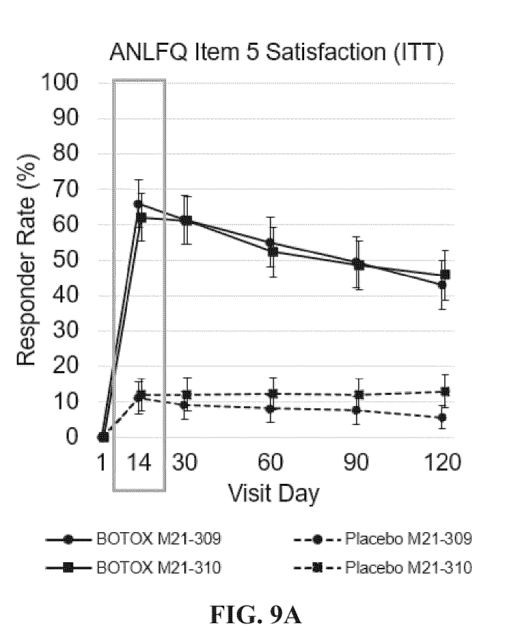

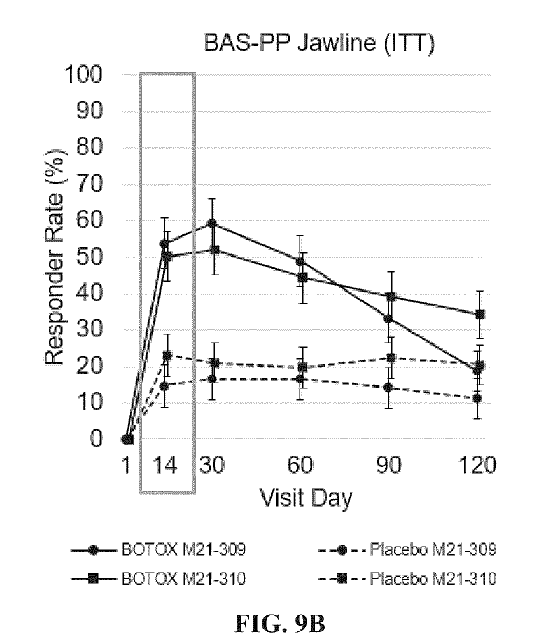

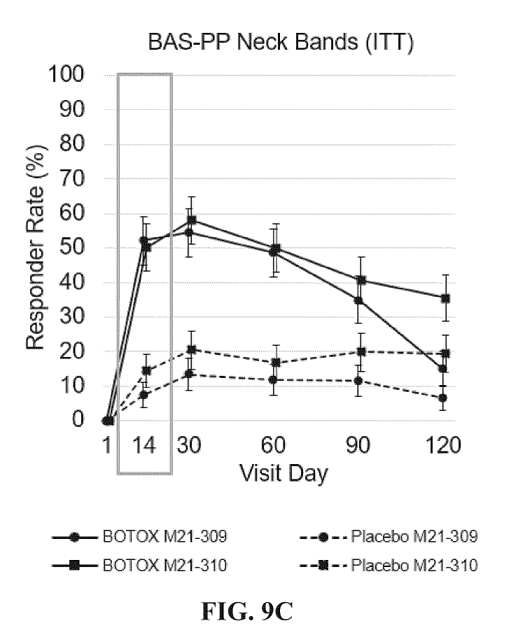

Based on ANLFQ: Satisfaction (Follow-up) Item 5 (satisfaction with the effect of treatment) at Day 14, higher percentages of BOTOX&-treated subjects reported being “Very Satisfied” or “Satisfied” with the effect of treatment (66.4% of subjects in Study 1 and 63.1% of subjects in Study 2) compared to placebo-treated subjects (10.1% in Study 1 and 11.5% in Study 2) [p<0.0001, for both studies](see FIG. 9A).

Based on the BAS-PP Item 2 (jawline) at Day 14, higher percentages of BOTOX&-treated subjects reported being “Not at all bothered” or “A little bothered” by the appearance of their jawline (55.3% of subjects in Study 1 and 49.8% of subjects in Study 2) compared to placebo-treated subjects (13.9%/in Study 1 and 21.6% in Study 2) [p<0.0001, for both studies](see FIG. 9B).

Based on the BAS-PP Item 1 (vertical neck bands) at Day 14, higher percentages of BOTOX®-treated subjects reported being “Not at all bothered” or “A little bothered” by the appearance of their vertical neck bands (52.2% of subjects in Study 1 and 50.7% of subjects in Study 2) compared to placebo-treated subjects (6.6% in Study 1 and 13.4% in Study 2) [p<0.0001, for both studies](see FIG. 9C).

The US (composite) and EU (coprimary) primary endpoints were met with statistical significance favoring BOTOX® over placebo (p-value<0.0001). All US and EU secondary endpoints were also met with statistical significance favoring BOTOX® over placebo at Day 14 (p-value<0.0001). Subjects treated with BOTOX® were more satisfied with the effect of treatment and less bothered by the appearance of their jawline and neck bands compared with placebo. Thus, BOTOX® treatment of 26 U to 36 U demonstrated significant improvement in platysma prominence.

Regarding safety, the majority of treatment-emergent adverse events (TEAEs) were mild in severity, and the frequency of TEAEs was similar across treatment groups (TEAE is defined as any AE with the onset that is after the first dose of study drug). No new safety signals were identified. The TEAE data are summarized in Table 10 below:

TABLE 10
Adverse Events: Overall Number of Subjects with Adverse Events (Safety Population).

| | M21-310 | | | M21-309 | | |
|---|---|---|---|---|---|---|
| | Placebo | BOTOX | Total | Placebo | BOTOX | Total |
| Safety population | N = 216 | N = 208 | N = 424 | N = 209 | N = 199 | N = 408 |
| Treatment-Emergent Adverse | 43 (19.9%) | 34 (16.3%) | 77 (18.2%) | 43 (21.5%) | 47 (23.6%) | 92 (22.5%) |
| Events (TEAE), n (%) | | | | | | |
| TEAE Related to Study | 7 (3.2%) | 8 (3.8%) | 15 (3.5%) | 10 (4.8%) | 6 (3.0%) | 16 (3.9%) |
| Treatment, n (%) | | | | | | |
| TEAE Related to Study | 6 (2.8%) | 7 (3.4%) | 13 (3.1%) | 10 (4.8%) | 6 (3.0%) | 16 (3.9%) |
| Procedure | | | | | | |
| TEAE Related to Study Drug | 1 (0.5%) | 2 (1.0%) | 3 (0.7%) | 3 (1.4%) | 3 (1.5%) | 6 (1.5%) |
| Mild | 6 (2.8%) | 8 (3.8%) | 14 (3.3%) | 9 (4.3%) | 6 (3.0%) | 15 (3.7%) |
| Moderate | 1 (0.5%) | 0 | 1 (0.2%) | 1 (0.5%) | 0 | 1 (0.2%) |
| Severe | 0 | 0 | 0 | 0 | 0 | 0 |
| Treatment-Emergent Serious | 1 (0.5%) | 0 | 1 (0.2%)* | 3 (1.4%) | 2 (1.0%) | 5 (1.2%)* |
| Adverse Events (TESAE), n (%) | | | | | | |
| TESAE Related to Study | 0 | 0 | 0 | 0 | 0 | 0 |
| Treatment | | | | | | |
| TEAE Leading to Death, n (%) | 0 | 0 | 0 | 0 | 0 | 0 |
| Adverse Events of Special | 0 | 0 | 0 | 0 | 0 | 0 |
| Interest (AESI), n (%) | | | | | | |
| Possible Distant Spread of Toxin | 0 | 0 | 0 | 1 (0.5%) | 0 | 1 (0.2%)** |
| (PDSOT), n (%) | | | | | | |
| All Deaths, n (%) | 0 | 0 | 0 | 0 | 0 | 0 |

*TESAE in the M21-310 study was adnexa uteri cyst; TESAEs in the M21-309 study were appendicitis, cellulitis, ophthalmic vascular thrombosis, subarachnoid haemorrhage, and thyroid cancer.
**Dyspnea as a symptom of allergic reaction in the placebo group was identified as a PDSOT. No evidence of distant spread of toxin.

The most common TEAEs reported were COVID-19, injection site bruising, and injection site haemorrhage (see Table 11 below, which shows the TEAEs reported at frequency ≥1.5% in the first study and at frequency ≥1.0% in the second study). The results show that the treatment emergent adverse events (TEAEs) in the BOTOX-treated subjects occurred at a similar or lesser rates than in the placebo group.

TABLE 11
Treatment-Emergent Adverse Events: Number (%) of Subjects by Preferred
Term (Safety Analysis Set).

| | M21-310 | | | M21-309 | | |
|---|---|---|---|---|---|---|
| | Placebo | BOTOX | Total | Placebo | BOTOX | Total |
| Preferred Term | N = 216 | N = 208 | N = 424 | N = 209 | N = 199 | N = 408 |
| Subjects with at least one | 43 (19.9%) | 34 (16.3%) | 77 (18.2%) | 45 (21.5%) | 47 (23.6%) | 92 (22.5%) |
| TEAE, n (%) | | | | | | |
| COVID-19 | 6 (2.8%) | 7 (3.4%) | 13 (3.1%) | 6 (2.9%) | 9 (4.5%) | 15 (3.7%) |
| Influenza like Illness | 3 (1.4%) | 3 (1.4%) | 6 (1.4%) | 0 | 0 | 0 |
| Nasopharyngitis | 4 (1.9%) | 2 (1.0%) | 6 (1.4%) | 1 (0.5%) | 3 (1.5%) | 4 (1.0%) |
| Injection Site Pain | 3 (1.4%) | 2 (1.0%) | 5 (1.2%) | 0 | 0 | 0 |
| Sinusitis | 0 | 3 (1.4%) | 3 (0.7%) | 1 (0.5%) | 3 (1.5%) | 4 (1.0%) |
| Injection Site Bruising | 1 (0.5%) | 2 (1.0%) | 3 (0.7%) | 5 (2.4%) | 2 (1.0%) | 7 (1.7%) |
| Injection Site Haemorrhage | 1 (0.5%) | 2 (1.0%) | 3 (0.7%) | 3 (1.4%) | 3 (1.5%) | 6 (1.5%) |
| Actinic Keratosis | 0 | 2 (1.0%) | 2 (0.5%) | 0 | 0 | 0 |

Most treatment-related TEAEs were associated with study procedure (see Table 12 below).

Treatment-Emergent Adverse Events Related to Study Treatment
According to Investigator.

| | M21-310 | | | M21-309 | | |
|---|---|---|---|---|---|---|
| System Organ Class | Placebo | BOTOX | Total | Placebo | BOTOX | Total |
| Preferred Term | N = 216 | N = 208 | N = 424 | N = 209 | N = 199 | N = 408 |
| Subjects with at least one treatment | 7 (3.2%) | 8 (3.8%) | 15 (3.5%) | 10 (4.8%) | 6 (3.0%) | 16 (3.9%) |
| related TEAE, n (%) | | | | | | |
| Gastrointestinal Disorders, n (%) | 0 | 1 (0.5%) | 1 (0.2%) | 0 | 0 | 0 |
| Oesophageal food impaction | 0 | 1 (0.5%) | 1 (0.2%) | 0 | 0 | 0 |
| General Disorders and Administration | 6 (2.8%) | 6 (2.9%) | 12 (2.8%) | 9 (4.3%) | 5 (2.5%) | 14 (3.4%) |
| Site Conditions, n (%)* | | | | | | |
| Injection Site Pain | 3 (1.4%) | 2 (1.0%) | 5 (1.2%) | 0 | 1 (0.5%) | 1 (0.2%) |
| Injection Site Bruising | 1 (0.5%) | 2 (1.0%) | 3 (0.7%) | 5 (2.4%) | 1 (0.5%) | 6 (1.5%) |
| Injection Site Haemorrhage | 1 (0.5%) | 2 (1.0%) | 3 (0.7%) | 3 (1.4%) | 3 (1.5%) | 6 (1.5%) |
| Injection Site Swelling | 1 (0.5%) | 0 | 1 (0.2%) | 0 | 0 | 0 |
| Musculoskeletal and Connective | 0 | 2 (1.0%) | 2 (0.5%) | 0 | 1 (0.5%) | 1 (0.2%) |
| Tissue Disorders, n (%) | | | | | | |
| Muscle Tightness | 0 | 1 (0.5%) | 1 (0.2%) | 0 | 1 (0.5%) | 1 (0.2%) |
| Musculoskeletal Stiffness | 0 | 1 (0.5%) | 1 (0.2%) | 0 | 0 | 0 |
| Nervous System Disorders, n (%) | 1 (0.5%) | 0 | 1 (0.2%) | 1 (0.5%) | 1 (0.5%) | 2 (0.5%) |
| Presyncope | 1 (0.5%) | 0 | 1 (0.2%) | 0 | 0 | 0 |
| Respiratory, Thoracic and Mediastinal | 1 (0.5%) | 0 | 1 (0.2%) | 0 | 0 | 0 |
| Disorders, n (%) | | | | | | |
| Oropharyngeal Pain | 1 (0.5%) | 0 | 1 (0.2%) | 0 | 0 | 0 |

*In the M21-309 trial, 1 (0.5%) patient from the placebo group had injection site hematoma, no patient from the BOTOX group had injection site hematoma. Thus, in total 1 (0.2%) patient in the M21-309 trial had injection site hematoma.

The majority of TEAEs were mild in severity and frequencies were balanced across treatment groups. The most common treatment-related AEs were associated with study procedure (injection site haemorrhage and injection site bruising), were mild in severity, and typically resolved in 7 days. There were no treatment-related severe TEAEs nor TESAEs reported. There were no TEAEs leading to study discontinuations or deaths and there was no evidence of distant spread of toxin. It should be noted that there was no report of any of the following adverse events of special interest (AESIs): aspiration, aspiration pneumonia, dry mouth, dysphagia, dyspnea, facial paresis and muscular weakness (i.e., neck muscle weakness).

The data show that BOTOX® treatment of 26 U to 36 U for platysma prominence was well-tolerated, with no new safety signals identified, and this treatment demonstrated a favorable benefit/risk profile.

In conclusion, results from the two pivotal studies support the safe and effective treatment of platysma prominence with a BOTOX® treatment of 26 U to 36 U for improvement in the appearance of Moderate (Grade 3) to Severe (Grade 4) platysma prominence in adults.

In pivotal studies, 3.6% (30/834) of subjects were 65 years of age or older. The responder rates appeared to be higher for subjects younger than age 65 than for subjects 65 years or older.

The safety of BOTOX® 26 U, 31 U, or 36 U was evaluated in a total of 466 BOTOX®-treated subjects and 481 placebo-treated subjects in double-blind, placebo-controlled clinical studies for the improvement of platysma prominence. There were no adverse reactions reported by ≥1% of BOTOX®-treated subjects and more frequent than in placebo-treated subjects.

### 7.3. Example 3: Use of a Botulinum Toxin Type A (BOTOX®) for the Treatment of Platysma Prominence

Upon exit from the first Phase 3 study described in Example 2, eligible subjects had the option to participate in an 8-month Phase 3 open-label extension study (see FIG. 4) to further assess long term safety and efficacy of a botulinum toxin type A purified neurotoxin complex (BOTOX®) for the treatment of platysma prominence. Thus, Day 1 of this Phase 3 open-label extension study is the exit day from the first Phase 3 clinical trial described in Example 2. The Phase 3 open-label extension study described in this example was similarly designed to the two Phase 3 clinical trials described in Example 2, but had a duration of 8 months, was open-labelled, and allowed the participants to receive up to 3 additional treatments with BOTOX® (botulinum toxin type A) (26 Units, 31 Units, or 36 Units) with an interval of no less than 3 months (84 days) from the previous study treatment. In these subjects, similar efficacy profiles were observed with each subsequent BOTOX® treatment for platysma prominence severity, treatment satisfaction, and bother due to the appearance of their jawline and vertical neck bands. Adverse events that occurred at <1% among 350 BOTOX®-treated subjects include mild dysphagia in 3 subjects (0.9%) and mild facial paresis in 2 subjects (0.6%). Specifically, adverse events of dysphagia and facial paresis did not occur during the placebo-controlled period (Cycle 1). Dysphagia was reported in 3 subjects (1.1%) in Cycle 2 and 1 subject (0.6%) in Cycle 3. Facial paresis was reported in 1 subject (0.4%) in Cycle 2 and 1 subject (1.9%) in Cycle 4. These adverse events were reported as mild in severity and generally resolved without intervention. No change was observed in the overall safety profile with repeat dosing up to 4 treatments with BOTOX®.

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed:

1. A method for improving the appearance of platysma prominence associated with platysma muscle activity to a human patient, comprising:
(A) quantifying the number of continuous vertical neck bands at maximum contraction on a first side of the human patient's neck, wherein a continuous vertical neck band is a vertical neck band that extends continuously from the human patient's jawline to a lower neck region; and
(B) administering a composition comprising a botulinum toxin type A to the first side of the human patient's neck determined as having only one or only two continuous vertical neck bands at maximum contraction.

2. The method of claim 1, wherein the composition is administered to the first side of the human patient's neck by injection of:
(i) a first jawline dose divided among 4 injection sites in the upper segment of platysma muscle, inferior and parallel to the patient's lower mandibular border on the first side; and
(ii) (a) a first neck band dose divided among 5 injection sites along the only continuous vertical neck band if the first side of the human patient's neck has only one continuous vertical neck band; or (b) a first neck band dose divided among 5 injection sites along a first continuous vertical neck band and a second neck band dose divided among 5 injection sites along a second continuous vertical neck band if the first side of the human patient's neck has only two continuous vertical neck bands.

3. The method of claim 1 or 2, wherein the first side of the human patient's neck has only one continuous vertical neck band, and the administering step delivers a total unilateral dose of about 13 units of botulinum toxin type A to the first side of the human patient's neck.

4. The method of claim 1 or 2, wherein the first side of the human patient's neck has only two continuous vertical neck bands, and the administering step delivers a total unilateral dose of about 18 units of botulinum toxin type A to the first side of the human patient's neck.

5. The method of any one of claims 2-4, wherein:
(1) the first jawline dose is about 8 units of botulinum toxin type A and wherein optionally each injection of the first jawline dose is about 2 units of botulinum toxin type A;
(2) the first neck band dose is about 5 units of botulinum toxin type A and wherein optionally each injection of the first neck band dose is about 1 unit of botulinum toxin type A; and/or
(3) the second neck band dose is about 5 units of botulinum toxin type A and wherein optionally each injection of the second neck band dose is about 1 unit of botulinum toxin type A.

6. The method of any one of claims 1-5, further comprising repeating the steps of quantifying and administering to a second side of the human patient's neck.

7. The method of claim 6, wherein the step of administering to the first side and the step of administering to the second side deliver a total bilateral dose selected from the group consisting of about 26 units, about 31 units, and about 36 units of botulinum toxin type A to the human patient's neck.

8. A method for improving the appearance of platysma prominence associated with platysma muscle activity to a human patient who has only one continuous vertical neck band at maximum contraction on a first side of the neck, comprising administering a composition comprising a botulinum toxin type A to the first side of the human patient's neck, by injection of;
(i) a first jawline dose divided among 4 injection sites in the upper segment of platysma muscle, inferior and parallel to the patient's lower mandibular border on the first side; and
(ii) a first neck band dose divided among 5 injection sites along the only continuous vertical neck band;
wherein a continuous vertical neck band is a vertical neck band that extends continuously from the human patient's jawline to a lower neck region.

9. The method of claim 8, wherein the administering step delivers a total unilateral dose of about 13 units of botulinum toxin type A to the first side of the human patient's neck.

10. The method of claim 8 or 9, wherein:
(1) the first jawline dose is about 8 units of botulinum toxin type A and wherein optionally each injection of the first jawline dose is about 2 units of botulinum toxin type A; and
(2) the first neck band dose is about 5 units of botulinum toxin type A and wherein optionally each injection of the first neck band dose is about 1 unit of botulinum toxin type A.

11. The method of any one of claims 8-10, further comprising repeating the step of administering to a second side of the human patient's neck, wherein the second side of the human patient's neck has only one continuous vertical neck band at maximum contraction.

12. The method of claim 11, wherein the step of administering to the first side and the step of administering to the second side deliver a total bilateral dose of about 26 units of botulinum toxin type A to the human patient's neck.

13. A method for improving the appearance of platysma prominence associated with platysma muscle activity to a human patient who has only two continuous vertical neck bands at maximum contraction on a first side of the neck, comprising administering a composition comprising a botulinum toxin type A to the first side of the human patient's neck, by injection of:
(i) a first jawline dose divided among 4 injection sites in the upper segment of platysma muscle, inferior and parallel to the patient's lower mandibular border on the first side;
(ii) a first neck band dose divided among 5 injection sites along a first continuous vertical neck band; and
(iii) a second neck band dose divided among 5 injection sites along a second continuous vertical neck band;
wherein a continuous vertical neck band is a vertical neck band that extends continuously from the human patient's jawline to a lower neck region, and wherein the administering step delivers a total unilateral dose range of about 15 units to about 20 units of botulinum toxin type A to the first side of the human patient's neck.

14. The method of claim 13, wherein the administering step delivers a total unilateral dose of about 18 units of botulinum toxin type A to the first side of the human patient's neck.

15. The method of claim 13 or 14, wherein:
(1) the first jawline dose is about 8 units of botulinum toxin type A and wherein optionally each injection of the first jawline dose is about 2 units of botulinum toxin type A;
(2) the first neck band dose is about 5 units of botulinum toxin type A and wherein optionally each injection of the first neck band dose is about 1 unit of botulinum toxin type A; and
(3) the second neck band dose is about 5 units of botulinum toxin type A and wherein optionally each injection of the second neck band dose is about 1 unit of botulinum toxin type A.

16. The method of any one of claims 13-15, further comprising repeating the step of administering to a second side of the human patient's neck, wherein the second side of the human patient's neck has only two continuous vertical neck bands at maximum contraction.

17. The method of claim 16, wherein the step of administering to the first side and the step of administering to the second side deliver a total bilateral dose of about 36 units of botulinum toxin type A to the human patient's neck.

18. A method for improving the appearance of platysma prominence associated with platysma muscle activity to a human patient who has only one continuous vertical neck band at maximum contraction on a first side of the neck and only two continuous vertical neck bands at maximum contraction on a second side of the neck, the method comprising administering a composition comprising a botulinum toxin type A to the human patient's neck, by injection of:
(i) a first jawline dose divided among 4 injection sites in the upper segment of platysma muscle, inferior and parallel to the patient's lower mandibular border on the first side;
(ii) a first neck band dose divided among 5 injection sites along the only continuous vertical neck band on the first side;
(iii) a second jawline dose divided among 4 injection sites in the upper segment of platysma muscle, inferior and parallel to the patient's lower mandibular border on the second side;
(iv) a first neck band dose divided among 5 injection sites along a first continuous vertical neck band on the second side; and
(v) a second neck band dose divided among 5 injection sites along a second continuous vertical neck band on the second side;
wherein a continuous vertical neck band is a vertical neck band that extends continuously from the human patient's jawline to a lower neck region.

19. The method of claim 18, wherein the first jawline dose is equal to the second jawline dose.

20. The method of claim 18, wherein the first jawline dose is not equal to the second jawline dose.

21. The method of any one of claims 18-20, wherein:
(1) the first jawline dose is about 8 units of botulinum toxin type A and wherein optionally each injection of the first jawline dose is about 2 units of botulinum toxin type A;
(2) the second jawline dose is about 8 units of botulinum toxin type A and wherein optionally each injection of the second jawline dose is about 2 units of botulinum toxin type A;
(3) the first neck band dose on the first side is about 5 units of botulinum toxin type A and wherein optionally each injection of the first neck band dose on the first side is about 1 unit of botulinum toxin type A;
(4) the first neck band dose on the second side is about 5 units of botulinum toxin type A and wherein optionally each injection of the first neck band dose on the second side is about 1 unit of botulinum toxin type A; and
(5) the second neck band dose on the second side is about 5 units of botulinum toxin type A and wherein optionally each injection of the second neck band dose on the second side is about 1 unit of botulinum toxin type A.

22. The method of any one of claims 18-21, wherein the administering step delivers a total bilateral dose of about 31 units botulinum toxin type A to the human patient's neck.

23. The method of any one of claims 1-22, wherein a cosmetic effect of the method is obtained within 14 days following the administration of the botulinum toxin type A, and wherein:
(1) the cosmetic effect comprises a physician-assessed or human patient-assessed platysma prominence scale grade change from moderate or severe prior to the administration of the botulinum toxin type A to minimal or mild following the administration of the botulinum toxin type A; wherein optionally the duration of the cosmetic effect ranges from about 30 days to about 120 days following the administration of the botulinum toxin type A or the duration of the cosmetic effect is at least 120 days; and wherein optionally the method is statistically better than a comparable method wherein a placebo is administered instead of the botulinum toxin type A at achieving a physician-assessed or human patient-assessed grade of minimal or mild on a platysma prominence scale at 14 days post-administration;
(2) the cosmetic effect comprises a physician-assessed and human patient-assessed 2-grade or better improvement on a platysma prominence scale from moderate or severe prior to the administration of the botulinum toxin type A to minimal or mild following the administration of the botulinum toxin type A;
(3) the cosmetic effect comprises a physician-assessed and human patient-assessed platysma prominence scale grade change from moderate or severe prior to the administration of the botulinum toxin type A to minimal or mild following the administration of the botulinum toxin type A; or
(4) the cosmetic effect comprises achieving a physician-assessed and human patient-assessed grade of minimal or mild and at least a 2-grade improvement from baseline on a platysma prominence scale; wherein optionally the duration of the cosmetic effect is at least 120 days or the duration of the cosmetic effect ranges from about 60 days to about 120 days following the administration of the botulinum toxin type A; and wherein optionally the method is statistically better than a comparable method wherein a placebo is administered instead of the botulinum toxin type A at achieving both physician-assessed and human patient-assessed grade of minimal or mild and at least a 2-grade improvement from baseline on a platysma prominence scale at 14 days post-administration;
wherein optionally the platysma prominence scale is a platysma band severity grading scale.

24. The method of any one of claims 1-23, wherein the concentration of the botulinum toxin type A in the composition is about 40 units per mL.

25. The method of any one of claims 1-24, wherein the botulinum toxin type A is a 900 kD botulinum neurotoxin serotype A (BoNT/A) complex.

26. The method of claim 25, wherein the botulinum toxin type A is onabotulinumtoxinA.

27. The method of claim 25 or 26, wherein the composition comprises a powdered BoNT/A pharmaceutical composition that has been reconstituted with normal saline, wherein prior to the reconstitution, the powdered BoNT/A pharmaceutical composition consists of the 900 kDa BoNT/A complex, human albumin, and sodium chloride.

28. The method of any one of claims 1-27, wherein the composition is a liquid.

29. The method of any one of claims 2-28, wherein the injections are not made deeper than about 0.5 inches into a muscle; and/or wherein the injections are administered superficially and intramuscularly with the needle perpendicular to the surface of the skin.

30. The method of any one of claims 1-29, wherein the human patient further has one or more non-continuous neck bands on the first side and/or the second side of the human patient's neck; and/or wherein no additional dose of the botulinum toxin type A is injected to the human patient's neck.

31. The method of any one of claims 1-30, wherein the method is repeated about every 12 weeks.

32. The method of any one of claims 1-31, wherein the method results in at least one incidence rate selected from the group consisting of:
(1) an incidence rate of less than 5% of treatment-emergent adverse effects (TEAEs) related to the composition comprising the botulinum toxin type A;
(2) an incidence rate of less than 1% of treatment-related severe TEAEs, and optionally no incidence of treatment-related severe TEAE;
(3) an incidence rate of less than 5% of adverse events of special interest (AESIs), and optionally no incidence of AESIs, wherein AESIs include aspiration, aspiration pneumonia, dry mouth, dysphagia, dyspnea, facial paresis, and muscular weakness;
(4) an incidence rate of less than 10% of injection site bruising;
(5) an incidence rate of less than 1% of neck muscle weakness, and optionally no incidence of neck muscle weakness;
(6) an incidence rate of less than 2% of dysphagia, and optionally no incidence of dysphagia; and
(7) an incidence rate of less than 5% of injection site hematoma, and optionally no incidence of injection site hematoma.

33. The method of any one of claims 2-32, wherein the method results in a reduced incidence of adverse effects as compared to a comparable method that comprises:
(1) injecting one or more neck band doses of the botulinum toxin type A to one or more neck bands of a human subject's neck but does not comprise injecting a jawline dose of the botulinum toxin type A to the human subject's neck;
(2) injecting one or more neck band doses of the botulinum toxin type A to one or more neck bands of a human subject's neck, wherein the one or more neck bands injected with the one or more neck band doses are not limited to continuous vertical neck bands that extend continuously from the human subject's jawline to the lower neck region; and/or
(3) injecting three or more neck band doses of the botulinum toxin type A to one side of a human subject's neck, with each neck band dose being injected along a different neck band.

34. The method of claim 33, wherein the adverse effect is selected from the group consisting of aspiration, aspiration pneumonia, dry mouth, dysphagia, dyspnea, facial paresis, muscular weakness, injection site bruising, and injection site hematoma.

35. The method of any one of claims 1-34, which is for improving the appearance of platysma bands associated with platysma muscle activity.

36. The method of any one of claims 1-35, wherein a continuous vertical neck band is a visible vertical neck band that extends continuously from the human patient's jawline to a lower neck region and results in blunting of the jawline.

37. The method of any one of claims 2-36, wherein the 4 injection sites in the upper segment of platysma muscle comprise an anterior injection site in line with the oral commissure and a posterior injection site anterior to the angle of the mandible.

38. A composition comprising a botulinum toxin type A for use in the method of any one of claims 1-37.

39. Use of a composition comprising a botulinum toxin type A for the manufacturing of a medicament for providing improvement in the appearance of platysma prominence associated with platysma muscle activity to a human patient in accordance with the method of any one of claims 1-37.

40. Use of a composition comprising a botulinum toxin type A substantially as described herein.

41. A composition for the improvement of the appearance of platysma prominence substantially as described herein.
